(19) United States
(12) Patent Application Publication
(10) Pub. No.: US 2003/0082590 A1
(43) Pub. Date: May 1, 2003

(54) Exponential nucleic acid amplification using nicking endonucleases

(75) Inventors: Jeffrey Van Ness, Claremont, CA (US); David J. Galas, Claremont, CA (US); Lori K. Van Ness, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Keck Graduate Institute, Claremont, CA

(21) Appl. No.: 10/197,626

(22) Filed: Jul. 15, 2002

Related U.S. Application Data
(60) Provisional application No. 60/345,445, filed on Jan. 2, 2002.
(60) Provisional application No. 60/331,687, filed on Nov. 19, 2001.
(60) Provisional application No. 60/305,637, filed on Jul. 15, 2001.

(51) Int. Cl. C12Q 1/68; C12P 19/34

(52) U.S. Cl. 435/6; 435/91.2

(57) ABSTRACT

The present invention provides methods and composition for exponential nucleic acid amplification using nicking agents. The invention is useful in many areas such as disease diagnosis, genetic variation detection and pre-mRNA alternative splicing analysis.

### BACKGROUND OF THE INVENTION

1. Field of the Invention

This invention relates to the field of molecular biology, more particularly to methods and compositions involving nucleic acids, and still more particularly to methods and compositions related to amplifying nucleic acids using a nicking agent.

2. Description of the Related Art

A number of methods have been developed for rapid amplification of nucleic acids. These include the polymerase chain reaction (PCR), ligase chain reaction (LCR), self-sustained sequence replication (3SR), nucleic acid sequence based amplification (NASBA), transcription-based amplification system (TAS), strand displacement amplification (SDA), and amplification with Qβ replicase. Most of the methods widely used for nucleic acid amplification, such as PCR, require cycles of different temperatures to achieve cycles of denaturation and reannealing. Other methods, although they may be performed isothermally, require multiple sets of primers (e.g., bumper primers of thermophilic SDA) or are based on transcription and/or reverse transcription, which is sensitive to RNA degradation (e.g., TAS, NASBA and 3SR). Accordingly, there is a long felt need in the art for a simpler and more efficient method for nucleic acid amplification.

The present invention fulfills this and related needs as described below.

### BRIEF SUMMARY OF THE INVENTION

In contrast to previously known techniques for amplification of nucleic acids, the present invention provides a method for nucleic acid amplification that does not require the use of multiple sets of oligonucleotide primers and is not transcription-based. In addition, the present invention can be carried out under an isothermal condition, thus avoiding the expenses associated with the equipment for providing cycles of different temperatures. The present invention may find utilities in various applications such as genetic variation detection, disease diagnosis, and genetic variation detection.

To this end, the present invention provides methods, compounds, and compositions including systems and arrays as summarized below:

A method for amplifying a nucleic acid molecule (A2), comprising: (A) providing an at least partially double-stranded nucleic acid molecule (N1) comprising at least one of (i) a sequence of the sense strand of a first nicking agent recognition sequence (NARS), and (ii) a sequence of the antisense strand of the first NARS; (B) amplifying a first single-stranded nucleic acid molecule (A1) in the presence of a nicking agent (NA) that recognizes the first NARS, a DNA polymerase, and one or more deoxynucleoside triphosphate(s), wherein the amplifying uses a portion of N1 as a template for the polymerase; (C) providing a second single-stranded nucleic acid molecule (T2) comprising, from 5′ to 3′: (i) a sequence of the sense strand of a second NARS, and (ii) a sequence that is at least substantially complementary to A1; and (D) amplifying a third single-stranded nucleic acid molecule (A2) in the presence of T2, A1, the first NA, a second NA that recognizes the second NARS, the DNA polymerase, and the deoxynucleoside triphosphate(s), wherein A2 is at least substantially complementary to A1 and wherein A1, A2 or both are at most 25 nucleotides in length.

A method for amplifying a nucleic acid molecule (A2), comprising: (A) forming a mixture comprising: (i) an at least partially double-stranded nucleic acid molecule (N1) comprising a sequence of an antisense strand of a first nicking agent recognition sequence (NARS); (ii) a single-stranded nucleic acid molecule (T2) comprising, from 3′ to 5′: (a) a sequence that is at least substantially identical to a portion of N1 located 5′ to the sequence of the antisense strand of the first NARS in N1; and (b) a sequence of a sense strand of a second NERS; and (iii) a first nicking agent (NA) that recognizes the first NARS, a second NA that recognizes the second NARS, a DNA polymerase, and one or more deoxynucleoside triphosphate(s); and (B) maintaining said mixture at conditions that exponentially amplify a single-stranded nucleic acid molecule (A2), wherein A2 is at most 25 nucleotides in length.

A method for amplifying a nucleic acid molecule (A2), comprising: (A) forming a mixture of (i) an at least partially double-stranded nucleic acid molecule (N1) comprising a sequence of a sense strand of a first nicking agent recognition sequence (NARS); (ii) a single-stranded nucleic acid molecule (T2) comprising, from 3′ to 5′: (a) a sequence that is at least substantially complementary to a portion of N1 located 3′ to the sense strand of the first NARS in N1, and (b) a sequence of a sense strand of a second NARS; and (iii) a first nicking agent (NA) that recognizes the first NARS, a second NA that recognizes the second NARS; a DNA polymerase; and one or more deoxynucleoside triphosphate(s); and (B) maintaining said mixture at conditions that amplify a single-stranded nucleic acid molecule (A2), wherein A2 is at most 25 nucleotides in length.

A tandem nucleic acid amplification system comprising a first primer extension means for amplifying a first nucleic acid (A1) and a second primer extension means for amplifying a second nucleic acid (A2), wherein (i) A1 is the initial primer for the second primer extension means for amplifying A2; (ii) both the first and second primer extension means are contained within a single reaction vessel and require the presence of a nicking agent (NA); (iii) A1, A2 or both are at most 25 nucleotides in length; and (iv) A2 is at least substantially complementary to A1.

A method for exponential amplification of a nucleic acid molecule A2 comprising (a) amplifying a nucleic acid molecule (A1) using a first template nucleic acid (T1) comprising the sequence of one strand of a first nicking agent recognition sequence (NARS) as a template by a primer extension reaction in the presence of a first nicking endonuclease (NA) that recognizes the first NARS and a first DNA polymerase; and (b) amplifying A2 using a second template nucleic acid (T2) comprising the sequence of the sense strand of a second NARS as a template and A1 as the initial primer by a primer extension reaction in the presence of a second NA and a second DNA polymerase. Preferably, A1, A2 or both are at most 25 nucleotides in length.

A method for identifying a gene variation in a genomic nucleic acid or cDNA molecule, wherein the genetic variation is located 5′ to a sequence of the antisense strand of a first nicking endonuclease recognition sequence (NERS) in the genomic nucleic acid or cDNA molecule, the method comprising: (A) forming a mixture comprising: (i) the genomic nucleic acid or cDNA molecule, (ii) a single-stranded nucleic acid molecule (T2) comprising from 3′ to 5′: (a) a sequence that is at least substantially identical to a portion of the genomic nucleic acid or cDNA molecule located 5′ to the sequence of the antisense strand of the first NERS, and (b) a sequence of the sense strand of a second NERS, and (iii) a first nicking endonuclease (NE) that recognizes the first NERS; a second NE that recognizes the second NERS, a DNA polymerase, and one or more deoxynucleoside triphosphate(s); (B) maintaining the mixture at conditions that exponentially amplify a single-stranded nucleic acid molecule (A2); and (C) characterizing A2 to identify the gene variation in the genomic nucleic acid or cDNA molecule.

A method for identifying a genetic variation at a defined location in a target nucleic acid, comprising (a) forming a mixture of a first oligonucleotide primer (ODNP), a second ODNP, and the target nucleic acid, wherein (i) if the target nucleic acid is a double-stranded nucleic acid having a first strand and a second strand, then the first ODNP comprises a nucleotide sequence of a sense strand of a first nicking endonuclease recognition sequence (NERS) and a nucleotide sequence at least substantially complementary to a nucleotide sequence of the first strand of the target nucleic acid located 3′ to the complement of the genetic variation, and the second ODNP comprises a nucleotide sequence at least substantially complementary to a nucleotide sequence of the second strand of the target nucleic acid located 3′ to the genetic variation and optionally comprises a sequence of one strand of a restriction endonuclease recognition sequence (RERS), or, (ii) if the target nucleic acid is a single-stranded nucleic acid, then the first ODNP comprises a nucleotide sequence of a sense strand of a first NERS and a nucleotide sequence at least substantially identical to a nucleotide sequence of the target nucleic acid located 5′ to the genetic variation, and the second ODNP comprises a nucleotide sequence at least substantially complementary to a nucleotide sequence of the target nucleic acid located 3′ to the genetic variation and optionally comprises a RERS; and (b) extending the first and the second ODNPs to produce an extension product comprising the nucleotide sequences of the first ODNP and the second ODNP; (c) optionally digesting the extension product of step (b) with a restriction endonuclease that recognizes the RERS to produce a digestion product; (d) amplifying a first single-stranded nucleic acid fragment (A1) using one strand of the extension product of step (b) or the digestion product of step (c) as a template in the presence of a nicking endonuclease (NE) that recognizes the first NERS; (e) providing a second single-stranded nucleic acid molecule (T2) to anneal to A1, T2 comprising, from 5′ to 3′: (i) a sequence of the sense strand of a second NERS, and (ii) a sequence at least substantially complementary to A1; (f) amplifying a third single-stranded nucleic acid fragment (A2) using A1 as a template; and (g) characterizing A2 to identify the genetic variation in the target nucleic acid. Preferably A1, A2 or both have at most 25 nucleotides.

A method for identifying a genetic variation at a defined location in a target nucleic acid, comprising: (a) forming a mixture of a first ODNP, a second ODNP, and the target nucleic acid, wherein (i) if the target nucleic acid is a double-stranded nucleic acid having a first strand and a second strand, then the first ODNP comprises a nucleotide sequence of one strand of a first restriction endonuclease recognition sequence (RERS) and a nucleotide sequence at least substantially complementary to a nucleotide sequence of the first strand of the target nucleic acid located 3′ to the complement of the genetic variation, and the second ODNP comprises a nucleotide sequence of one strand of a second RERS and a nucleotide sequence at least substantially complementary to a nucleotide sequence of the second strand of the target nucleic acid located 3′ to the genetic variation; or, (ii) if the target nucleic acid is a single-stranded nucleic acid, then the first ODNP comprises a nucleotide sequence of one strand of a first RERS and a nucleotide sequence at least substantially identical to a nucleotide sequence of the target nucleic acid located 5′ to the complement of the genetic variation, and the second ODNP comprises a sequence of one strand of a second RERS and a nucleotide sequence at least substantially complementary to a nucleotide sequence of the target nucleic acid located 3′ to the genetic variation; (b) extending the first and the second ODNPs in the presence of deoxyribonucleoside triphosphates and at least one modified deoxyribonucleoside triphosphate to produce an extension product comprising both the first and the second RERSs; (c) exponentially amplifying single-stranded nucleic acid fragments using the extension product of step (b) as a template in the presence of restriction endonucleases (REs) that recognize the first RERS and the second RERS, wherein the single-stranded nucleic acid fragment is no more than 25 nucleotides in length; and (d) characterizing at least one of the single-stranded fragments of step (c) to identify the genetic variation.

A method for identifying a genetic variation at a defined location in a target nucleic acid, comprising: (a) forming a mixture of a first ODNP, a second ODNP, and the target nucleic acid, wherein (i) if the target nucleic acid is a double-stranded nucleic acid having a first strand and a second strand, then the first ODNP comprises a nucleotide sequence of one strand of a first restriction endonuclease recognition sequence (RERS) and a nucleotide sequence at least substantially complementary to a nucleotide sequence of the first strand of the target nucleic acid located 3′ to the complement of the genetic variation, and the second ODNP comprises a nucleotide sequence of one strand of a second RERS and a nucleotide sequence at least substantially complementary to a nucleotide sequence of the second strand of the target nucleic acid located 3′ to the genetic variation; or, (ii) if the target nucleic acid is a single-stranded nucleic acid, then the first ODNP comprises a nucleotide sequence of one strand of a first RERS and a nucleotide sequence at least substantially identical to a nucleotide sequence of the target nucleic acid located 5′ to the complement of the genetic variation, and the second ODNP comprises a sequence of one strand of a second RERS and a nucleotide sequence at least substantially complementary to a nucleotide sequence of the target nucleic acid located 3′ to the genetic variation; (b) extending the first and the second ODNPs in the presence of deoxyribonucleoside triphosphates and at least one modified deoxyribonucleoside triphosphate to produce an extension product comprising both the first and the second RERSs; (c) amplifying a first single-stranded nucleic acid fragment using one strand of the extension product of step (b) as a template in the presence of restriction endonucleases (REs) that recognize the first RERS and the second RERS; (d) providing a second single-stranded nucleic acid molecule (T2) to anneal to A1, T2 comprising, from 5′ to 3′: (i) a sequence of the sense strand of a third RERS, and (ii) a sequence at least substantially complementary to A1; (e) amplifying a third single-stranded nucleic acid fragment (A2) using A1 as a template; and (f) characterizing at least one of the single-stranded fragments of step (c) to identify the genetic variation.

A method for identifying a genetic variation at a defined location in a target nucleic acid, comprising (a) forming a mixture of a first oligonucleotide primer (ODNP), a second ODNP and the target nucleic acid, wherein (i) if the target nucleic acid is a double-stranded nucleic acid having a first strand and a second strand, then the first ODNP comprises a nucleotide sequence at least substantially complementary to a nucleotide sequence of the first strand of the target nucleic acid located 3′ to the complement of the genetic variation, and the second ODNP comprises a nucleotide sequence at least substantially complementary to a nucleotide sequence of the second strand of the target nucleic acid located 3′ to the genetic variation, or, (ii) if the target nucleic acid is a single-stranded nucleic acid, then the first ODNP comprises a nucleotide sequence at least substantially identical to a nucleotide sequence of the target nucleic acid located 5′ to the genetic variation, and the second ODNP comprises a nucleotide sequence at least substantially complementary to a nucleotide sequence of the target nucleic acid located 3′ to the genetic variation, where the first and the second ODNPs each further comprise a nucleotide sequence of the sense strand of a nicking endonuclease recognition sequence (NERS); (b) extending the first and the second ODNPs to produce an extension product comprising two NERSs; (c) exponentially amplifying single-stranded nucleic acid fragments using the extension product of step (b) as a template in the presence of one or more nicking endonucleases (NEs) that recognizes the NERS(s), wherein the single-stranded nucleic acid fragments have no more than 25 nucleotides; and (d) characterizing at least one of the single-stranded fragments of step (c) to thereby identify the genetic variation.

A method for identifying a genetic variation at a defined location in a target nucleic acid, comprising (a) forming a mixture of a first oligonucleotide primer (ODNP), a second ODNP and the target nucleic acid, wherein (i) if the target nucleic acid is a double-stranded nucleic acid having a first strand and a second strand, then the first ODNP comprises a nucleotide sequence at least substantially complementary to a nucleotide sequence of the first strand of the target nucleic acid located 3′ to the complement of the genetic variation, and the second ODNP comprises a nucleotide sequence at least substantially complementary to a nucleotide sequence of the second strand of the target nucleic acid located 3′ to the genetic variation, or, (ii) if the target nucleic acid is a single-stranded nucleic acid, then the first ODNP comprises a nucleotide sequence at least substantially identical to a nucleotide sequence of the target nucleic acid located 5′ to the genetic variation, and the second ODNP comprises a nucleotide sequence at least substantially complementary to a nucleotide sequence of the target nucleic acid located 3′ to the genetic variation, where the first and the second ODNPs each further comprise a nucleotide sequence of a sense strand of a nicking endonuclease recognition sequence (NERS); (b) extending the first and the second ODNPs to produce an extension product comprising two NERSs; (c) amplifying a first single-stranded nucleic acid fragment using one strand of the extension product of step (b) as a template in the presence of one or more nicking endonucleases (NEs) that recognizes the NERS(s); (d) providing a second single-stranded nucleic acid molecule (T2) to anneal to A1, T2 comprising, from 5′ to 3′: (i) a sequence of the sense strand of a NERS, and (ii) a sequence at least substantially complementary to A1; (e) amplifying a third single-stranded nucleic acid fragment (A2) using A1 as a template; and (f) characterizing the single-stranded fragment of step (c) to thereby identify the genetic variation. Preferably, A1, A2 or both have at most 25 nucleotides.

A method for determining the presence or the absence of a target nucleic acid in a sample, comprising: (A) forming a mixture comprising: (i) the nucleic acid molecules of the sample; (ii) a first single-stranded nucleic acid molecule (T1) comprising from 3′ to 5′: (a) a first sequence that is at least substantially complementary to the target nucleic acid, (b) a sequence of the antisense strand of a first nicking agent recognition sequence (NARS), and (c) a second sequence having at most 25 nucleotides; (iii) a second single-stranded nucleic acid molecule (T2) comprising from 3′ to 5′: (a) a sequence that is at least substantially identical to the second sequence of T1, and (b) a sequence of the sense strand of a second NARS; and (iv) a first nicking endonuclease (NA) that recognizes the first NARS, a second NA that recognizes the second NARS, a DNA polymerase, and one or more deoxynucleoside triphosphate(s); (B) maintaining the mixture at conditions that exponentially amplify a single-stranded nucleic acid molecule (A2) if the target nucleic acid is present in the sample; and (C) detecting the presence or the absence of A2 to determine the presence, or the absence, of the target nucleic acid in the sample.

A method for determining the presence or the absence of a target nucleic acid in a sample, comprising: (A) form a mixture comprising: (i) the nucleic acid molecules of the sample; (ii) a first single-stranded nucleic acid molecule (T1) comprising from 3′ to 5′: (a) a sequence that is at least substantially complementary to the target nucleic acid, and (b) a sequence of the sense strand of a first nicking agent recognition sequence (NARS), (iii) a second single-stranded nucleic acid molecule (T2) comprising from 3′ to 5′: (a) a sequence that is at least substantially complementary to the sequence of T1 that is located 3′ to the sequence of the sense strand of the first NARS, and (b) a sequence of the sense strand of a second NARS; and (iv) a first nicking endonuclease (NA) that recognizes the first NARS, a second NA that recognizes the second NARS, a DNA polymerase, and one or more deoxynucleoside triphosphate(s); (B) maintaining the mixture at conditions that amplify a single-stranded nucleic acid molecule (A2) if the target nucleic acid is present in the sample, where A2 (i) is at least substantially identical to the target nucleic acid, and (ii) preferably has at most 25 nucleotides; and (C) detecting the presence or the absence of A2 to determine the presence, or the absence, of the target nucleic acid in the sample.

A method for determining the presence or absence of a target nucleic acid that comprises a first nicking endonuclease recognition sequence (NERS) in a sample, the method comprising: (A) forming a mixture comprising: (i) the nucleic acid molecules of the sample, (ii) a single-stranded nucleic acid molecule (T2) comprising from 3′ to 5′: (a) a sequence that is at least substantially identical to a portion of the target nucleic acid molecule located 5′ to the sequence of the antisense strand of the first NERS, and (b) a sequence of the sense strand of a second NERS, and (iii) a first nicking endonuclease (NE) that recognizes the first NERS; a second NE that recognizes the second NERS, a DNA polymerase, and one or more deoxynucleoside triphosphate(s); (B) maintaining the mixture at conditions that exponentially amplify a single-stranded nucleic acid molecule (A2) if the target nucleic acid is present in the sample; and (C) detecting the presence or absence of A2 to determine the presence or absence of the target nucleic acid in the sample.

A method for determining the presence or absence of a target nucleic acid that comprises a first nicking endonuclease recognition sequence (NERS) in a sample, the method comprising: (A) forming a mixture comprising: (i) the target nucleic acid molecule, (ii) a first single-stranded nucleic acid molecule (T1) that is substantially identical to one strand of the target nucleic acid and comprise a sequence of the antisense strand of the first NERS, (iii) a second single-stranded nucleic acid molecule (T2) comprising from 3′ to 5′: (a) a sequence that is at least substantially identical to a portion of T1 located 5′ to the sequence of the antisense strand of the first NERS, and (b) a sequence of the sense strand of a second NERS, and (iv) a first nicking endonuclease (NE) that recognizes the first NERS; a second NE that recognizes the second NERS, a DNA polymerase, and one or more deoxynucleoside triphosphate(s); (B) maintaining the mixture at conditions that exponentially amplify a single-stranded nucleic acid molecule (A2) if the target nucleic acid is present in the sample; and (C) detecting the presence or absence of A2 to determine the presence or absence of the target nucleic acid in the sample.

A method for determining the presence or absence of a target nucleic acid in a sample, comprising: (A) forming a mixture of a first oligonucleotide primer (ODNP), a second ODNP, and the nucleic acid molecules of the sample, wherein (i) if the target nucleic acid is a double-stranded nucleic acid having a first strand and a second strand, the first ODNP comprises a nucleotide sequence of a sense strand of a first restriction endonuclease recognition sequence (RERS) and a nucleotide sequence at least substantially complementary to a first portion of the first strand of the target nucleic acid, and the second ODNP comprises a nucleotide sequence at least substantially complementary to a second portion of the second strand of the target nucleic acid and comprises a sequence of the sense strand of a second RERS, the second portion being located 3′ to the complement of the first portion in the second strand of the target nucleic acid, or, (ii) if the target nucleic acid is a single-stranded nucleic acid, the first ODNP comprises a nucleotide sequence of a sense strand of a first RERS and a nucleotide sequence at least substantially identical to a first portion of the target nucleic acid, and the second ODNP comprises a nucleotide sequence at least substantially complementary to a second portion of the target nucleic acid and comprises a sequence of the sense strand of a second RERS, the second portion being located 5′ to the first portion in the target nucleic acid; (B) maintaining the mixture at conditions that, if the target nucleic acid is present in the sample, exponentially amplify a single-stranded nucleic acid fragment (A2) in the presence of restriction endonucleases (REs) that recognize the first RERS and the second RERS, deoxyribonucleoside triphosphates and at least one modified deoxyribonucleoside triphosphate, and a DNA polymerase, wherein A2 is no more than 25 nucleotides in length; and (C) detecting the presence or absence of A2 to determine the presence or absence of the target nucleic acid in the sample.

A method for determining the presence or absence of a target nucleic acid in a sample, comprising: (A) forming a mixture of a first oligonucleotide primer (ODNP), a second ODNP, and the nucleic acid molecules of the sample, wherein (i) if the target nucleic acid is a double-stranded nucleic acid having a first strand and a second strand, the first ODNP comprises a nucleotide sequence of a sense strand of a first nicking endonuclease recognition sequence (NERS) and a nucleotide sequence at least substantially complementary to a first portion of the first strand of the target nucleic acid, and the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target nucleic acid, or, (ii) if the target nucleic acid is a single-stranded nucleic acid, the first ODNP comprises a sequence of a sense strand of a first NERS and a nucleotide sequence at least substantially identical to a first portion of the target nucleic acid, and the second ODNP comprises a nucleotide sequence at least substantially complementary to a second portion of the target nucleic acid and comprises a sequence of the sense strand of a second NERS, the second portion being located 5′ to the first portion in the target nucleic acid; (B) maintaining the mixture at conditions that, if the target nucleic acid is present in the sample, exponentially amplify a single-stranded nucleic acid fragment (A2) in the presence of nicking endonucleases (NEs) that recognize the first NERS and the second NERS, deoxyribonucleoside triphosphates, and a DNA polymerase, wherein A2 is preferably no more than 25 nucleotides in length; and (C) detecting the presence or absence of A2 to determine the presence or absence of the target nucleic acid in the sample.

A method for determining the presence or absence of a target nucleic acid in a sample, comprising: (A) forming a mixture of a first oligonucleotide primer (ODNP), a second ODNP, and the nucleic acid molecule of the sample, wherein (i) if the target nucleic acid is a double-stranded nucleic acid having a first strand and a second strand, the first ODNP comprises a nucleotide sequence of a sense strand of a first nicking endonuclease recognition sequence (NERS) and a nucleotide sequence at least substantially complementary to a first portion of the first strand of the target nucleic acid, and the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target nucleic acid, or, (ii) if the target nucleic acid is a single-stranded nucleic acid, the first ODNP comprises a nucleotide sequence of a sense strand of a first NERS and a nucleotide sequence at least substantially identical to a first portion of the target nucleic acid, and the second ODNP comprises a nucleotide sequence at least substantially complementary to a second portion of the target nucleic acid and comprises a sequence of the sense strand of a second NERS, the second portion being located 5′ to the first portion in the target nucleic acid; (B) subjecting the mixture to conditions that, if the target nucleic acid is present in the sample, (i) extend the first and the second ODNPs to produce an extension product comprising both the first and the second NERSs; (ii) amplify a first single-stranded nucleic acid fragment (A1) using one strand of the extension product of step (b) as a template in the presence of one more nicking endonucleases (NEs) that recognizes the first and the second NERSs; (iii) in the presence of a second single-stranded nucleic acid molecule (T2) capable of annealing to A1, amplify a third single-stranded nucleic acid fragment (A2) using A1 as a template, wherein A1, A2 or both have at most 25 nucleotides, and wherein T2 comprising, from 5′ to 3′: (a) a sequence of the sense strand of a third NERS, and (b) a sequence at least substantially complementary to A1; and (C) detecting the presence or absence of A2 to determine the presence or absence of the target nucleic acid in the sample.

A method for determining the presence or absence of a target nucleic acid in a sample, comprising: (A) forming a mixture comprising: (i) the nucleic acid molecules of the sample, and (ii) a single-stranded nucleic acid probe (T1) that comprises, from 3′ to 5′, a sequence that is at least substantially complementary to the 5′ portion of the target nucleic acid, and a sequence of the antisense strand of a first nicking agent recognition sequence (NARS), (B) separating the probe molecules that have hybridized to the target nucleic acid, if any, from those that have not hybridized to the target nucleic acid; (C) performing an amplification reaction in the presence of the probe molecules that have hybridized to the target nucleic acid, if any, and a first nicking agent (NA) that recognizes the first NARS; (D) providing a single-stranded nucleic acid molecule (T2) comprising, from 5′ to 3′: (i) a sequence of the sense strand of a second NARS, and (ii) a sequence that is at least substantially identical to the portion of the first single-stranded nucleic acid probe located 5′ to the sequence of the antisense strand of the first NARS, (E) performing an amplification reaction in the presence of a second NA that recognizes the second NARS; (F) detecting the presence or absence of the amplification product of step (E) to determine the presence or absence of the target nucleic acid in the sample.

A method for determining the presence or absence of a target nucleic acid in a sample, comprising: (A) forming a mixture comprising: (i) the nucleic acid molecules of the sample, and (ii) a partially double-stranded nucleic acid probe that comprises: (a) a sequence of a sense strand of a first NARS, a sequence of an antisense of the first NARS, or both; and (b) a 5′ overhang in the strand that the strand itself or an extension product thereof contains a nicking site (NS) nickable by a first nicking agent (NA) that recognizes the first NARS, or a 3′ overhang in the strand that neither the strand nor an extension product thereof contains the NS, wherein an overhang comprises a nucleic acid sequence at least substantially complementary to the target nucleic acid; (B) separating the probe molecules that have hybridized to the target nucleic acid, if any, from those that have not hybridized to the target nucleic acid; (C) performing an amplification reaction in the presence of the probe molecules that have hybridized to the target nucleic acid, if any, and a first nicking agent (NA) that recognizes the first NARS; (D) providing a single-stranded nucleic acid molecule (T2) comprising, from 5′ to 3′: (i) a sequence of the sense strand of a second NARS, and (ii) a sequence that is at least substantially identical to the portion of the nucleic acid probe located 5′ to the sequence of the antisense strand of the first NARS, (E) performing an amplification reaction in the presence of a second NA that recognizes the second NARS; (F) detecting the presence or absence of the amplification product of step (E) to determine the presence or absence of the target nucleic acid in the sample.

A method for determining the presence or absence of a junction between two specific exons in a cDNA molecule, comprising: (A) providing an at least partially double-stranded nucleic acid molecule (N1) comprising: (i) at least one of a sequence of the sense strand of a first nicking agent recognition sequence (NARS) and a sequence of the antisense strand of the first NARS, and (ii) at least one strand of a portion of the cDNA molecule, the portion being suspected to contain the junction between the two exons; (B) amplifying a first single-stranded nucleic acid molecule (A1) in the presence of a nicking agent (NA) that recognizes the first NARS, a DNA polymerase, and one or more deoxynucleoside triphosphate(s), wherein the amplifying uses the portion of the cDNA as a template for the polymerase; (C) providing a second single-stranded nucleic acid molecule (T2) comprising, from 5′ to 3′: (i) a sequence of the sense strand of a second NARS, and (ii) a sequence that is at least substantially complementary to A1; (D) amplifying a third single-stranded nucleic acid molecule (A2) in the presence of T2, A1, the first NA, a second NA that recognizes the second NARS, the DNA polymerase, and the deoxynucleoside triphosphate(s), wherein A2 is at least substantially complementary to A1; and (E) detecting and/or characterizing A2 to determine the presence or absence of the junction in the cDNA molecule.

A method for determining the presence or absence of a junction between two exons in a cDNA molecule, wherein the junction, if present, is located 5′ to a sequence of the antisense strand of a first nicking endonuclease recognition sequence (NERS) in the cDNA molecule, the method comprising: (A) forming a mixture comprising: (i) the cDNA molecule, (ii) a single-stranded nucleic acid molecule (T2) comprising from 3′ to 5′: (a) a sequence that is at least substantially identical to a portion of the cDNA molecule located 5′ to the sequence of the antisense strand of the first NERS, and (b) a sequence of the sense strand of a second NERS, and (iii) a first nicking endonuclease (NE) that recognizes the first NERS; a second NE that recognizes the second NERS, a DNA polymerase, and one or more deoxynucleoside triphosphate(s); (B) maintaining the mixture at conditions that exponentially amplify a single-stranded nucleic acid molecule (A2); and (C) characterizing A2 to determine the presence or absence of the junction in the cDNA molecule.

A method for determining the presence or absence of a junction between an upstream exon (Exon A) and a downstream exon (Exon B) of a gene in a cDNA molecule, comprising: (A) forming a mixture of a first oligonucleotide primer (ODNP), a second ODNP, and the cDNA molecule, wherein (i) the first ODNP comprises a sequence at least substantially complementary to a portion of the antisense strand of Exon A near the 5′ terminus of Exon A in the antisense strand, (ii) the second ODNP comprises a sequence at least substantially complementary to a portion of the sense strand of Exon B near the 5′ terminus of Exon B in the sense strand, and (iii) at least one of the first ODNP and the second ODNP further comprises a sequence of a sense strand of a first nicking agent recognition sequence (NARS); and (B) performing a first amplification reaction in the presence of a nicking agent (NA) that recognizes the first NARS under the conditions that amplify a first single-stranded nucleic acid (A1) if both Exon A and Exon B are present in the cDNA; (C) providing a second single-stranded nucleic acid molecule (T2) comprising, from 5′ to 3′: (i) a sequence of the sense strand of a second NARS, and (ii) a sequence at least substantially complementary to A1; (D) performing a second amplification reaction in the presence of a second NA that recognizes the second NARS under the conditions that amplify a third single-stranded nucleic acid fragment (A2) using A1 as a template if both Exon A and Exon B are present in the cDNA molecule; and (G) detecting and/or characterizing A2 to determine the presence or absence of the junction between Exon A and Exon B in the cDNA molecule.

A method for determining the presence or absence of a junction between an upstream exon (Exon A) and a downstream exon (Exon B) of a gene in a cDNA molecule, comprising: (A) forming a mixture of a first oligonucleotide primer (ODNP), a second ODNP, and the cDNA molecule, wherein (i) the first ODNP comprises: (a) a sequence at least substantially complementary to a portion of the antisense strand of Exon A near the 5′ terminus of Exon A in the antisense strand, and (b) a sequence of the sense strand of a first nicking agent recognition sequence (NARS); and (ii) the second ODNP comprises (a) a sequence at least substantially complementary to a portion of the sense strand of Exon B near the 5′ terminus of Exon B in the sense strand, and (b) a sequence of the sense strand of a second NARS; (B) performing a first amplification reaction in the presence of a first nicking agent (NA) that recognizes the first NARS and a second NA that recognizes the second NARS under the conditions that amplify a first single-stranded nucleic acid (A1) if both Exon A and Exon B are present in the cDNA; (C) providing a second single-stranded nucleic acid molecule (T2) comprising, from 5′ to 3′: (i) a sequence of the sense strand of a third NARS, and (ii) a sequence at least substantially complementary to A1; (D) performing a second amplification reaction in the presence of a third NA that recognizes the second NARS under the conditions that amplify a third single-stranded nucleic acid fragment (A2) using A1 as a template if both Exon A and Exon B are present in the cDNA molecule; and (E) detecting and/or characterizing A2 to determine the presence or absence of the junction between Exon A and Exon B in the cDNA molecule.

A method for determining the presence or absence of a junction between an upstream exon (Exon A) and a downstream exon (Exon B) of a gene in a cDNA molecule, comprising: (A) forming a mixture of a first oligonucleotide primer (ODNP), a second ODNP, and the cDNA molecule, wherein (i) the first ODNP comprises (a) a sequence at least substantially complementary to a portion of the antisense strand of Exon A near the 5′ terminus of Exon A in the antisense strand, and (b) a sequence of the sense strand of a first nicking agent recognition sequence (NARS); and (ii) the second ODNP comprises: (a) a sequence at least substantially complementary to a portion of the sense strand of Exon B near the 5′ terminus of Exon B in the sense strand, and (b) a sequence of the sense strand of a second NARS; (B) maintaining the mixture at conditions that, if both Exon A and Exon B are present in the cDNA molecule, exponentially amplify a single-stranded nucleic acid fragment (A2); and (C) detecting and/or characterizing A2 to determine the presence or absence of the junction between Exon A and Exon B in the cDNA molecule.

The following criteria may be used, alone or in any combination, to further describe the methods of the present invention as outlined above and elsewhere herein, where these criteria are exemplary only and other criteria as may be set forth elsewhere herein may also be utilized to further describe a method of the present invention: the first NARS is identical to the second NARS; the first nicking agent is the same as the second nicking agent; any one or more NARSs in a method is a NERS; both the first and the second NAs are a nicking endonuclease (NE); the NE is N.BstNB I; the NE is N.Alw I; both the first and the second NEs are N.BstNB I; at least one of the first or second nicking agents is a nicking endonuclease; both the first and the second NAs are restriction endonucleases (REs); the first, second and third NARSs (when three NARSs are specified in an embodiment of the invention) are identical to each other; each of the first, second and third NARSs is recognized by a nicking endonuclease; at least one of a first, second and third NARS is recognized by a nicking endonuclease; any one, or any two, or any three, or any four, or any five, or any six, or any seven, or any eight, or any nine, or any ten etc. steps of the method (e.g., steps (A), (B), (C) and (D), or e.g., steps (a) through (j)) are performed in a single vessel; the amplification of a single-stranded nucleic acid fragment is performed under isothermal conditions; each amplification reaction is performed at one or more temperatures within the range of 5° C.-70° C.; each amplification reaction is performed at, or at about, 60° C.; each amplification reaction is performed at temperatures between a highest temperature and a lowest temperature, where the highest temperature is within 20° C. of the lowest temperature; each amplification reaction is performed at temperatures between a highest temperature and a lowest temperature, where the highest temperature is within 15° C. of the lowest temperature; each amplification reaction is performed at temperatures between a highest temperature and a lowest temperature, where the highest temperature is within 10° C. of the lowest temperature; each amplification reaction is performed at temperatures between a highest temperature and a lowest temperature, where the highest temperature is within 5° C. of the lowest temperature; N1 includes the nucleotide sequence of the sense strand of the first NERS; N1 includes the nucleotide sequence of the antisense strand of the first NERS; both the first and the second NAs are restriction endonucleases (REs); N1 is provided by annealing a trigger oligonucleotide primer (ODNP) and a single-stranded nucleic acid (T1), where T1 includes the nucleotide sequence of either the sense strand or the antisense strand of the first NERS; when N1 is provided by annealing a trigger oligonucleotide primer (ODNP) to a single-stranded target nucleic acid (T1) that comprises, from 5′ to 3′: (A) a sequence of an antisense strand of the first NARS; and (B) a sequence that is at least substantially complementary to at least a portion of the trigger ODNP, then the sequence (B) of T1 is exactly complementary to at least a portion of the trigger ODNP; T1 is substantially identical to T2; the 3′ terminus of T2 is linked to a phosphate group; the 3′ terminus of T1 is linked to a phosphate group; T1 is exactly identical to T2; T1 is neither substantially nor exactly identical to T2; the nucleotide sequence of T2 that is at least substantially identical to a portion of N1 located 5′ to the antisense strand of the NARS in N1 is, in fact, exactly identical to a portion of N1 located 5′ to the antisense strand of the first NARS; when T3 includes a sequence that is at least substantially identical to at least a portion of the template sequence of T2, then in one embodiment T3 includes a sequence that is exactly identical to at least a portion of the template sequence T2; A2 includes a nucleotide sequence that is at least substantially identical to a nucleotide sequence in A1; A2 includes a nucleotide sequence that is exactly identical to a nucleotide sequence in A1; A1 includes a nucleotide sequence that is at least substantially identical to a nucleotide sequence in A2; A1 includes a nucleotide sequence that is exactly identical to a nucleotide sequence in A2; A2 and A1 are identical; A1 is substantially identical to A2; A2 is substantially identical to A1; A1 is exactly identical to A2; A1 is neither substantially nor exactly identical to A2; A1 is substantially identical to the trigger ODNP; A1 is exactly identical to the trigger ODNP; A2 is substantially identical to the trigger ODNP; A2 is exactly identical to the trigger ODNP; A1 is at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25 nucleotides in length, while additionally, or alternatively, A1 is no more than 40, or no more than 39, or no more than 38, or no more than 37, or no more than 36, or no more than 35, or no more than 34, or no more than 33, or no more than 32, or no more than 31, or no more than 30, or no more than 29, or no more than 28, or no more than 27, or no more than 26, or no more than 25, or no more than 24, or no more than 23, or no more than 22, or no more than 21, or no more than 20, or no more than 19, or no more than 18, or no more than 17, or no more than 16, or no more than 15, or no more than 14, or no more than 13, or no more than 12, or no more than 11, or no more than 10 nucleotides in length, where any stated upper limit on the nucleotide length of A1 may be combined with any stated lower limit on the nucleotide length of A1, so that A1 may be, for example, from 8 to 24 nucleotides in length, or from 12 to 17 nucleotides in length; A2 is at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25 nucleotides in length, while additionally, or alternatively, A2 is no more than 40, or no more than 39, or no more than 38, or no more than 37, or no more than 36, or no more than 35, or no more than 34, or no more than 33, or no more than 32, or no more than 31, or no more than 30, or no more than 29, or no more than 28, or no more than 27, or no more than 26, or no more than 25, or no more than 24, or no more than 23, or no more than 22, or no more than 21, or no more than 20, or no more than 19, or no more than 18, or no more than 17, or no more than 16, or no more than 15, or no more than 14, or no more than 13, or no more than 12, or no more than 11, or no more than 10 nucleotides in length, where any stated upper limit on the nucleotide length of A2 may be combined with any stated lower limit on the nucleotide length of A2, so that A2 may be, for example, from 8 to 24 nucleotides in length, or from 12 to 17 nucleotides in length; the initial number of T2 molecules is more than the initial number of T1 molecules; N1 is derived from a genomic DNA; N1 is a portion of a genomic DNA; the target nucleic acid is one strand of a denatured double-stranded nucleic acid; the target nucleic acid is one strand of double-stranded genomic nucleic acid or cDNA; the target nucleic acid is an RNA molecule; the target nucleic acid is derived from nucleic acid obtained from a bacterium; the target nucleic acid is derived from nucleic acid obtained from a virus; the target nucleic acid is derived from nucleic acid obtained from a fungus; the target nucleic acid is derived from nucleic acid derived from a parasite; the trigger ODNP is one strand of double-stranded genomic nucleic acid or cDNA; the trigger ODNP is an RNA molecule; the trigger ODNP is derived from nucleic acid obtained from a bacterium; the trigger ODNP is derived from nucleic acid obtained from a virus; the trigger ODNP is derived from nucleic acid obtained from a fungus; the trigger ODNP is derived from nucleic acid derived from a parasite; at least one of the deoxynucleoside triphosphate(s) is labeled; at least one of the deoxynucleoside triphosphate(s) is linked to a radiolabel; at least one of the deoxynucleoside triphosphate(s) is linked to an enzyme label at least one of the deoxynucleoside triphosphate(s) is linked to a fluorescent dye that functions as a label; at least one of the deoxynucleoside triphosphate(s) is linked to digoxidenin which functions as a label; at least one of the deoxynucleoside triphosphate(s) is linked to biotin; the same DNA polymerase type is used in all of the steps of a method; the DNA polymerase is 5′→3′ exonuclease deficient; the DNA polymerase is 5′→3′ exonuclease deficient and selected from exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme, where any two or more of the listed DNA polymerases may be combined to form a group from which the DNA polymerase used in a method of the invention is selected, e.g., the 5′→3′ exonuclease deficient DNA polymerase is exo⁻ Bst polymerase, exo⁻ Bca polymerase, exo⁻ Vent polymerase, 9° Nm™ DNA polymerase or exo⁻ Deep Vent polymerase; the DNA polymerase has a strand displacement activity; each amplification reaction is performed in the presence of a strand displacement facilitator; a strand displacement facilitator is used during amplification, where the strand displacement facilitator is selected from the group BMRF1 polymerase accessory subunit, adenovirus DNA-binding protein, herpes simplex viral protein ICP8, single-stranded DNA binding proteins, phage T4 gene 32 protein, calf thymus helicase, and trehalose, where the invention provides that any two or more of the listed facilitators may be combined to form a group from which a facilitator is selected in order to perform an embodiment of the present invention; the strand displacement facilitator is trehalose.

Any of the methods of the present invention may, and preferably does, include the step of detecting an amplified nucleic acid, e.g., detecting the formation, either qualitatively or quantitatively, of A2. In one embodiment, the detection is performed at least partially by a technique selected from luminescence spectroscopy or spectrometry, fluorescence spectroscopy or spectrometry, mass spectrometry, liquid chromatography, fluorescence polarization, and electrophoresis, where any two, three, four, or more members of the listed techniques may be grouped together so as to form a group of techniques from which the techniques utilized in an embodiment of the present invention may be selected, e.g., the detection may performed by mass spectrometry or liquid chromatography. In one embodiment, the detection entails the use of a fluorescence-intercalating agent that specifically binds to double-stranded nucleic acid.

In further aspects, the present invention provides:

A composition comprising: (A) a first at least partially double-stranded nucleic acid molecule (e.g., N1 or H1 as described herein) of which one strand comprises from 5′ to 3′: (i) a sequence at most 25 nucleotides in length, and (ii) a sequence of the antisense strand of a first nicking agent recognition sequence (NARS); and (B) a second at least double-stranded nucleic acid molecule (e.g., N2 or H2 as described herein) of which one strand comprises, from 5′ to 3′: (i) a sequence of the sense strand of a second NARS, and (ii) a sequence at least substantially identical to a sequence located 5′ to the sequence of the antisense strand of the first NARS in the first nucleic acid molecule.

A composition comprising: (a) a first at least partially double-stranded nucleic acid molecule (e.g., N1 or H1 as described herein) of which one strand comprises a sequence of the sense strand of a first nicking agent recognition sequence (NARS); and (b) a second at least double-stranded nucleic acid molecule (e.g., N2 or H2 as described herein) of which one strand comprises from 5′ to 3′: (i) a sequence of the sense strand of a second NARS, and (ii) a sequence that is at least substantially complementary to a sequence located 3′ to the sequence of the sense strand of the first NARS in the first nucleic acid molecule, wherein in the presence of a nicking agent that recognizes the first NARS, a DNA polymerase, and one or more nucleoside triphospates, a single-stranded nucleic acid fragment amplified using the first nucleic acid molecule as a template has at most 25 nucleotides.

In these compositions, the following additional criteria and/or components may be used to describe the compositions, as well as criteria set forth above in connection with the methods of the invention: the composition further comprises a first NA that recognizes the first NARS and a second NA that recognizes the second NARS; the composition further comprises a nicking agent that recognizes both the first and second NARSs; the composition further comprises a nicking endonuclease (NE) that recognizes both the first and the second NERSs; the composition further comprises a nicking agent (NA) that recognizes both the first and the second NARSs; the composition further comprises N.BstNB I; the composition further comprises a DNA polymerase; the composition further comprises a DNA polymerase that is 5′→3′ exonuclease deficient; the composition further comprises a DNA polymerase selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme; the composition further comprises a DNA polymerase with strand displacement activity; the composition further comprises a strand displacement facilitator; the composition further comprises a strand displacement facilitator selected from the group BMRF1 polymerase accessory subunit, adenovirus DNA-binding protein, herpes simplex viral protein ICP8, single-stranded DNA binding proteins, phage T4 gene 32 protein, calf thymus helicase, and trehalose, where one or more members of this group may be combined to form a group from which the facilitator is selected in an embodiment of the invention; the composition includes trehalose; the composition includes a labeled deoxynucleoside triphosphate. The composition includes a labeled oligonucleotide that is at least substantially complementary to a sequence located 5′ to the sequence of the antisense strand of the second NARS in T2. The composition includes a fluorescent intercalating agent.

In any of the methods or compounds or compositions of the present invention that include a NARS, the NARS may contain a, i.e., one or more, mismatched nucleotides. In other words, one or more of the nucleotide base pairs that form the NARS may not be hybridized according to the conventional Watson-Crick base pairing rules. However, when mismatched nucleotides are present in the NARS, then at least all of the nucleotides that are necessary to form the sense strand of the NARS are present. In one embodiment, there are no mismatched base pairs present in a NARS, and furthermore every nucleotide present in the NARS is paired with a nucleotide in the complementary strand according to conventional Watson-Crick base pairing rules. In one embodiment, an NARS comprises a mismatched base pair. In one embodiment, there is one mismatched base pair in the NARS, while in another embodiment there are two mismatched base pairs in the NARS, while in another embodiment all of the base pairs that form the NARS are mismatched, while in another embodiment, n−1 of the base pairs that form the NARS are mismatched, where n base pairs form the NARS. In one embodiment where the invention utilizes both first and second NARSs, the mismatches present in the first NARS are also present in the second NARS. In one embodiment where the invention utilizes both first and second NARSs, the mismatches present in the first NARS are not also present in the second NARS. In one embodiment where the invention utilizes both first and second NARSs, the first NARS does not contain mismatched base pairs, however the second NARS does contain one or more mismatched base pairs. In one embodiment, there is an unmatched nucleotide in the NARS. In another embodiment, all of the nucleotides that form the sense sequence of the NARS are unmatched. In another embodiment, the NARS comprises an unmatched nucleotide.

Also, in the methods, compounds and compositions of the present invention, one or more of the nucleic acid molecules may be immobilized to a solid support. Typically, this immobilization allows for the ready separation of hybridized vs. unhybridized material. In various embodiments: the first ODNP is immobilized; the second ODNP is immobilized; both the first and second ODNPs are immobilized; the target nucleic acid is immobilized; T2, or each T2, is immobilized; immobilization is to a solid support via covalent attachment. Suitable solid supports are made from materials such as silica, plastic and metal.

These and other aspects of the present invention will become evident upon reference to the following detailed description and attached drawings.

### BRIEF DESCRIPTION OF THE DRAWINGS

FIG. 1 is a schematic diagram of the major steps of the first amplification reaction of a tandem amplification system of the present invention.

FIG. 2 is a schematic diagram of the major steps of the second amplification reaction of a tandem amplification system of the present invention.

FIG. 3 is a schematic diagram of the major steps of an exemplary method for nucleic acid amplification according to the present invention, where the recognition sequence of N.BstNB I is used as an exemplary NARS, the first template (T1) comprises a sequence of the antisense strand of the NARS (i.e., 5′-GACTC-3′), and the second template (T2) comprises a sequence of the sense strand of the NARS (i.e., 5′-GAGTC-3′).

FIG. 4 is a schematic diagram of the major steps of an exemplary method for nucleic acid amplification according to the present invention, wherein the recognition sequence of N.BstNB I is used as an exemplary NARS, both the first template (T1) and the second template (T2) comprise a sequence of the sense strand of the NARS (i.e., 5′-GAGTC-3′).

FIG. 5 shows a schematic diagram of the major steps for preparing an initial nucleic acid molecule N1 by annealing a trigger ODNP derived from a genomic DNA to a first template T1 and subsequent amplification of a single-stranded nucleic acid molecule A1. The trigger ODNP is prepared by digesting the genomic DNA and then denaturing the digested genomic DNA.

FIG. 6 shows a schematic diagram of the major steps for preparing an initial nucleic acid molecule N1 from a genomic DNA and subsequent amplification of a single-stranded nucleic acid molecule A1. The genomic DNA comprises a nicking endonuclease recognition sequence. The N1 molecule is produced by annealing one strand of the genomic DNA fragment with a portion of the other strand of the genomic DNA fragment (i.e., T1).

FIG. 7 shows a schematic diagram of the major steps for preparing an initial nucleic acid molecule N1 from a genomic DNA and subsequent amplification of a single-stranded nucleic acid molecule A1. The genomic DNA comprises a nicking agent recognition sequence. The N1 molecule is produced by annealing one strand of the genomic DNA fragment to a first template (T1) that is complementary to the strand of the genomic DNA at its 3′ portion, but not at its 5′ portion.

FIG. 8 shows a schematic diagram of the major steps for preparing an initial nucleic acid molecule N1 from a genomic DNA and subsequent amplification of a nucleic acid molecule A1. The genomic DNA comprises a nicking agent recognition sequence and a restriction endonuclease recognition sequence. A nicking endonuclease recognition sequence recognizable by a nicking endonuclease that nicks outside its recognition sequence is used as an exemplary nicking agent recognition sequence.

FIG. 9 shows a schematic diagram of the major steps for preparing an initial nucleic acid molecule N1 from a target nucleic acid using two oligonucleotide primers and subsequent amplification of a nucleic acid molecule A1. One primer comprises a sequence of a sense strand of a NERS while the other comprises one strand of a Type IIs restriction endonuclease recognition sequence (TRERS).

FIG. 10a shows a schematic diagram of the major steps for preparing initial nucleic acid molecules N1a and N1b using two ODNPs and subsequent amplification of nucleic acid molecules A1a and A1b. In this exemplary embodiment, both ODNPs comprise a sequence of the sense strand of a NERS.

FIG. 10b shows a schematic diagram of the major steps for amplifying nucleic acid molecules A2a and A2b using A1a and A1b of FIG. 10a as respective templates. In this exemplary embodiment, the first and second primers of FIG. 10a anneal to A1b and A1a, respectively, to form N2b and N2a molecules.

FIG. 11 shows a schematic diagram of the major steps for preparing an initial nucleic acid molecule N1 in an exemplary embodiment using two ODNPs and subsequent amplification of a nucleic acid molecule A1. Both ODNPs comprise a sequence of one strand of a RERS. The amplification is performed in the presence of an α-thio deoxynucleoside triphosphate, which is used as an exemplary modified deoxynucleoside triphosphate.

FIG. 12 shows a schematic diagram of a method for detecting an immobilized target nucleic acid using a partially double-stranded initial nucleic acid molecule N1 that comprises a NERS.

FIG. 13 shows a schematic diagram of a method for detecting an immobilized target nucleic acid using a single-stranded nucleic acid molecule T1 that comprises a sequence of the antisense strand of a NERS.

FIG. 14 shows a schematic diagram of the major steps for preparing an initial nucleic acid molecule N1 from a target nucleic acid and subsequent amplification of a single-stranded nucleic acid molecule A1. The target nucleic acid comprises a restriction endonuclease recognition sequence and a potential genetic variation.

FIG. 15 shows a schematic diagram of the major steps for preparing an initial nucleic acid molecule N1 from a target nucleic acid and subsequent amplification of single-stranded nucleic acid molecule A1. The target nucleic acid comprises a nicking agent recognition sequence, a restriction endonuclease recognition sequence, and a genetic variation between the two recognition sequences.

FIG. 16 shows a schematic diagram of the major steps for preparing an initial nucleic acid molecule N1 from a target nucleic acid using two primers and subsequent amplification of a nucleic acid molecule A1. The target nucleic acid comprises a genetic variation (“X”). The first primer comprises a sequence of the sense strand of a nicking endonuclease recognition sequence, whereas the second primer comprises a sequence of one strand of a type IIs restriction endonuclease recognition sequence.

FIG. 17 shows a schematic diagram of the major steps for preparing initial nucleic acid molecules (N1a and N1b) from a target nucleic acid using two primers and subsequent amplification of nucleic acid molecules A1a and A1b. The target nucleic acid comprises a genetic variation (“X”). Both primers comprise a sequence of the sense strand of a nicking endonuclease recognition sequence.

FIG. 18 shows a schematic diagram of the major steps for preparing initial nucleic acid molecules (N1a and N1b) from a target nucleic acid using two primers and subsequent amplification of nucleic acid molecules A1a and A1b. The target nucleic acid comprises a genetic variation (“X”). Both primers comprise a sequence of one strand of a restriction endonuclease recognition sequence.

FIG. 19 shows that a schematic diagram of the major steps for preparing an initial nucleic acid molecule (N1) from a target cDNA and subsequent amplification of a nucleic acid molecule (A1). The target cDNA comprises a restriction endonuclease recognition sequence and a location suspected to be a specific exon-exon junction.

FIG. 20 shows that a schematic diagram of the major steps for preparing an initial nucleic acid molecule (N1) from a target cDNA and subsequent amplification of a nucleic acid molecule (A1). The target cDNA comprises a nicking endonuclease recognition sequence, a restriction endonuclease recognition sequence, and a location suspected to be a specific exon-exon junction between the two recognition sequences.

FIGS. 21A and 21B show schematic diagrams of the process for preparing an initial nucleic acid molecule (N1) from a target cDNA and subsequent amplification of a nucleic acid molecule (A1). The target cDNA comprises Exon A and Exon B that is directly downstream to Exon A (FIG. 21A), or Exon A, Exon B, and a sequence between Exon A and Exon B (FIG. 21B).

FIG. 22 shows a schematic diagram of the major steps for preparing an initial nucleic acid molecule (N1) from a target cDNA using two primers and subsequent amplification of a nucleic acid molecule (A1). The target cDNA comprises exon A and exon B. The first primer comprises a sequence of the sense strand of a nicking endonuclease recognition sequence and anneal to a portion of the antisense strand of exon A. The second primer comprises a sequence of the antisense strand of a type IIs restriction endonuclease recognition sequence and anneals to a portion of the sense strand of exon B.

FIG. 23 shows a schematic diagram of the major steps for preparing initial nucleic acid molecules (N1a and N1b) from a target cDNA using two primers and subsequent amplification of a nucleic acid molecule (A1a and A1b). The target cDNA comprises exon A and exon B. Both primers comprise a sequence of the sense strand of a nicking endonuclease recognition sequence. The first primer anneals to a portion of the antisense strand of exon A, whereas the second primer anneals to a portion of the sense strand of exon B.

FIG. 24 shows a schematic diagram of the major steps for preparing initial nucleic acid molecules (N1a and N1b) from a target cDNA using two primers and subsequent amplification of a nucleic acid molecule (A1a and A1b). The target cDNA comprises exon A and exon B. Both primers comprise a sequence of one strand of a restriction endonuclease recognition sequence. The first primer anneals to a portion of the antisense strand of exon A, whereas the second primer anneals to a portion of the sense strand of exon B.

FIG. 25 shows a schematic diagram of a method for detecting the presence of a target nucleic acid in using an immobilized T1 molecule that comprises a sequence of the sense strand of a NARS and a sequence that is at least substantially complementary to the 3′ portion of the target nucleic acid.

FIG. 26 shows a schematic diagram of a method for detecting the presence of a target nucleic acid in using an immobilized T1 molecule that comprises a sequence of the sense strand of a NARS and is at least substantially complementary to the target nucleic acid.

### DETAILED DESCRIPTION OF THE INVENTION

The present invention provides simple and efficient methods and kits for exponential amplification of nucleic acids using nicking agents. The amplification can be carried out isothermally and is not transcription-based. These methods and kits are useful in many areas, such as in genetic variation detection, pathogen or disease diagnosis, and differential splicing analysis.

### A. Conventions/Definitions

Prior to providing a more detailed description of the present invention, it may be helpful to an understanding thereof to define conventions and provide definitions as used herein, as follows. Additional definitions are also provided throughout the description of the present invention.

The terms “3” and “5” are used herein to describe the location of a particular site within a single strand of nucleic acid. When a location in a nucleic acid is “3′ to” or “3′ of” a reference nucleotide or a reference nucleotide sequence, this means that the location is between the 3′ terminus of the reference nucleotide or the reference nucleotide sequence and the 3′ hydroxyl of that strand of the nucleic acid. Likewise, when a location in a nucleic acid is “5′ to” or “5′ of” a reference nucleotide or a reference nucleotide sequence, this means that it is between the 5′ terminus of the reference nucleotide or the reference nucleotide sequence and the 5′ phosphate of that strand of the nucleic acid. Further, when a nucleotide sequence is “directly 3′ to” or “directly 3′ of” a reference nucleotide or a reference nucleotide sequence, this means that the nucleotide sequence is immediately next to the 3′ terminus of the reference nucleotide or the reference nucleotide sequence. Similarly, when a nucleotide sequence is “directly 5′ to” or “directly 5′ of” a reference nucleotide or a reference nucleotide sequence, this means that the nucleotide sequence is immediately next to the 5′ terminus of the reference nucleotide or the reference nucleotide sequence.

A “naturally occurring nucleic acid” refers to a nucleic acid molecule that occurs in nature, such as a full-length genomic DNA molecule or an mRNA molecule.

An “isolated nucleic acid molecule” refers to a nucleic acid molecule that is not identical to any naturally occurring nucleic acid or to that of any fragment of a naturally occurring genomic nucleic acid spanning more than three separate genes.

As used herein, “nicking” refers to the cleavage of only one strand of a fully double-stranded nucleic acid molecule or a double-stranded portion of a partially double-stranded nucleic acid molecule at a specific position relative to a nucleotide sequence that is recognized by the enzyme that performs the nicking. The specific position where the nucleic acid is nicked is referred to as the “nicking site” (NS).

A “nicking agent” (NA) is an enzyme that recognizes a particular nucleotide sequence of a completely or partially double-stranded nucleic acid molecule and cleaves only one strand of the nucleic acid molecule at a specific position relative to the recognition sequence. Nicking agents include, but are not limited to, a nicking endonuclease (e.g., N.BstNB I) and a restriction endonuclease (e.g., Hinc II) when a completely or partially double-stranded nucleic acid molecule contains a hemimodified recognition/cleavage sequence in which one strand contains at least one derivatized nucleotide(s) that prevents cleavage of that strand (i.e., the strand that contains the derivatized nucleotide(s)) by the restriction endonuclease.

A “nicking endonuclease” (NE), as used herein, refers to an endonuclease that recognizes a nucleotide sequence of a completely or partially double-stranded nucleic acid molecule and cleaves only one strand of the nucleic acid molecule at a specific location relative to the recognition sequence. Unlike a restriction endonuclease (RE), which requires its recognition sequence to be modified by containing at least one derivatized nucleotide to prevent cleavage of the derivatized nucleotide-containing strand of a fully or partially double-stranded nucleic acid molecule, a NE typically recognizes a nucleotide sequence composed of only native nucleotides and cleaves only one strand of a fully or partially double-stranded nucleic acid molecule that contains the nucleotide sequence.

As used herein, “native nucleotide” refers to adenylic acid, guanylic acid, cytidylic acid, thymidylic acid or uridylic acid. A “derivatized nucleotide” is a nucleotide other than a native nucleotide.

The nucleotide sequence of a completely or partially double-stranded nucleic acid molecule that a NA recognizes is referred to as the “nicking agent recognition sequence” (NARS). Likewise, the nucleotide sequence of a completely or partially double-stranded nucleic acid molecule that a NE recognizes is referred to as the “nicking endonuclease recognition sequence” (NERS). The specific sequence that a RE recognizes is referred to as the “restriction endonuclease recognition sequence” (RERS). A “hemimodified RERS,” as used herein, refers to a double-stranded RERS in which one strand of the recognition sequence contains at least one derivatized nucleotide (e.g., α-thio deoxynucleotide) that prevents cleavage of that strand (i.e., the strand that contains the derivatized nucleotide within the recognition sequence) by a RE that recognizes the RERS.

In certain embodiments, a NARS is a double-stranded nucleotide sequence where each nucleotide in one strand of the sequence is complementary to the nucleotide at its corresponding position in the other strand. In such embodiments, the sequence of a NARS in the strand containing a NS nickable by a NA that recognizes the NARS is referred to as a “sequence of the sense strand of the NARS” or a “sequence of the sense strand of the double-stranded NARS,” while the sequence of the NARS in the strand that does not contain the NS is referred to as a “sequence of the antisense strand of the NARS” or a “sequence of the antisense strand of the double-stranded NARS.”

Likewise, in the embodiments where a NERS is a double-stranded nucleotide sequence of which one strand is exactly complementary to the other strand, the sequence of a NERS located in the strand containing a NS nickable by a NE that recognizes the NERS is referred to as a “sequence of a sense strand of the NERS” or a “sequence of the sense strand of the double-stranded NERS,” while the sequence of the NERS located in the strand that does not contain the NS is referred to a “sequence of the antisense strand of the NERS” or a “sequence of the antisense strand of the double-stranded NERS.” For example, the recognition sequence and the nicking site of an exemplary nicking endonuclease, N.BstNB I, are shown below with “▾” to indicate the cleavage site and N to indicate any nucleotide:

▾
5′-GAGTCNNNNN-3′
3′-CTCAGNNNNN-5′

The sequence of the sense strand of the N.BstNB I recognition sequence is 5′-GAGTC-3′, whereas that of the antisense strand is 5′-GACTC-3′.

Similarly, the sequence of a hemimodified RERS in the strand containing a NS nickable by a RE that recognizes the hemimodified RERS (i.e., the strand that does not contain any derivatized nucleotides) is referred to as “the sequence of the sense strand of the hemimodified RERS” and is located in “the sense strand of the hemimodified RERS” of a hemimodified RERS-containing nucleic acid, while the sequence of the hemimodified RERS in the strand that does not contain the NS (i.e., the strand that contains derivatized nucleotide(s)) is referred to as “the sequence of the antisense strand of the hemimodified RERS” and is located in “the antisense strand of the hemimodified RERS” of a hemimodified RERS-containing nucleic acid.

In certain other embodiments, a NARS is an at most partially double-stranded nucleotide sequence that has one or more nucleotide mismatches, but contains an intact sense strand of a double-stranded NARS as described above. According to the convention used herein, in the context of describing a NARS, when two nucleic acid molecules anneal to one another so as to form a hybridized product, and the hybridized product includes a NARS, and there is at least one mismatched base pair within the NARS of the hybridized product, then this NARS is considered to be only partially double-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′
3′-NNNNN-5′

where N indicates any nucleotide, and N at one position may or may not be identical to N at another position, however there is at least one mismatched base pair within this recognition sequence. In this situation, the NARS will be characterized as having at least one mismatched nucleotide.

In certain other embodiments, a NARS is a partially or completely single-stranded nucleotide sequence that has one or more unmatched nucleotides, but contains an intact sense strand of a double-stranded NARS as described above. According to the convention used herein, in the context of describing a NARS, when two nucleic acid molecules (i.e., a first and a second strand) anneal to one another so as to form a hybridized product, and the hybridized product includes a nucleotide sequence in the first strand that is recognized by a NA, i.e., the hybridized product contains a NARS, and at least one nucleotide in the sequence recognized by the NA does not correspond to, i.e., is not across from, a nucleotide in the second strand when the hybridized product is formed, then there is at least one unmatched nucleotide within the NARS of the hybridized product, and this NARS is considered to be partially or completely single-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′
3′-N₀₋₄-5′

(where “N” indicates any nucleotide, 0-4 indicates the number of the nucleotides “N,” a “N” at one position may or may not be identical to a “N” at another position), which contains the sequence of the sense strand of the double-stranded recognition sequence of N.BstNB I. In this instance, at least one of G, A, G, T or C is unmatched, in that there is no corresponding nucleotide in the complementary strand. This situation arises, e.g., when there is a “loop” in the hybridized product, and particularly when the sense sequence is present, completely or in part, within a loop.

As used herein, the phrase “amplifying a nucleic acid molecule” or “amplification of a nucleic acid molecule” refers to the making of two or more copies of the particular nucleic acid molecule. “Exponentially amplifying a nucleic acid molecule” or “exponential amplification of a nucleic acid molecule” refers to the amplification of the particular nucleic acid molecule by a tandem amplification system that comprises two or more nucleic acid amplification reactions. In such a system, the amplification product from the first amplification reaction functions as an initial amplification primer for the second nucleic acid amplification reaction. As used herein, the term “nucleic acid amplification reaction” refers to the process of making more than one copy of a nucleic acid molecule (A) using a nucleic acid molecule (T) that comprises a sequence complementary to the sequence of nucleic acid molecule A as a template. According to the present invention, both the first and the second nucleic acid amplification reactions employ nicking and primer extension reactions.

An “initial primer,” as used herein, is a primer that anneals to a template nucleic acid and initiates a nucleic acid amplification reaction. An initial primer must function as a primer for an initial primer extension, but need not be the primer for any subsequent primer extensions. For instance, assume that a primer A1 anneals to a portion of a template nucleic acid T1 that comprises the sequence of a sense strand of a NARS at a location 3′ to the sense strand of the NARS. In the presence of a DNA polymerase, the 3′ terminus of A1 is extended using T1 as a template to produce a double-stranded or partially double-stranded nucleic acid molecule (H1) that contains the double-stranded NARS. In the presence of a NA that recognizes the NARS, H1 is nicked in the strand complementary to the initial primer A1. The strand that contains the 3′ terminus at the nicking site, not the initial primer A1, may function as a primer for subsequent primer extensions in the presence of the NA and the DNA polymerase. A1 is regarded as an initial primer although it functions as a primer only for the first primer extension, but not the subsequent primer extensions.

A “trigger oligonucleotide primer (ODNP)” is an ODNP that functions as a primer in the first nucleic acid amplification reaction of a tandem nucleic acid amplification system. It triggers exponential amplification of a nucleic acid molecule in the presence of the other required components of the system (e.g., DNA polymerase, NA, deoxynucleoside triphosphates, the template for the first amplification reaction (T1), and the template for the second amplification reaction (T2)). In certain embodiments, when the template for the first amplification reaction (T1) comprises the sequence of one strand of a NARS, the trigger ODNP may comprise the sequence of the other strand of the NARS. A trigger ODNP may be derived from a target nucleic acid or may be chemically synthesized.

A first nucleic acid molecule (“first nucleic acid”) is “derived from” or “originates from” another nucleic acid molecule (“second nucleic acid”) if the first nucleic acid is either a digestion product of the second nucleic acid, or an amplification product using a portion of the second nucleic acid molecule or the complement thereof as a template. The first nucleic acid molecule must comprise a sequence that is exactly identical to, or exactly complementary to, at least a portion of the second nucleic acid.

A first nucleic acid sequence is “at least substantially identical” to a second nucleic acid sequence when the complement of the first sequence is able to anneal to the second sequence in a given reaction mixture (e.g., a nucleic acid amplification mixture). In certain preferred embodiments, the first sequence is “exactly identical” to the second sequence, that is, the nucleotide of the first sequence at each position is identical to the nucleotide of the second sequence at the same position, and the first sequence is of the same length as the second sequence.

A first nucleic acid sequence is “at least substantially complementary” to a second nucleic acid sequence when the first sequence is able to anneal to the second sequence in a given reaction mixture (e.g., a nucleic acid amplification mixture). In certain preferred embodiments, the first sequence is “exactly or completely complementary” to the second sequence, that is, each nucleotide of the first sequence is complementary to the nucleotide of the second sequence at its corresponding position, and the first sequence is of the same length as the second sequence.

As used herein, a nucleotide in one strand (referred to as the “first strand”) of a double-stranded nucleic acid located at a position “corresponding to” another position (e.g., a defined position) in the other strand (referred to as the “second strand”) of a double-stranded nucleic acid refers to the nucleotide in the first strand that is complementary to the nucleotide at the corresponding position in the second strand. Likewise, a position in one strand (referred to as the “first strand”) of a double-stranded nucleic acid corresponding to a nicking site within the other strand (referred to as the “second strand”) of a double-stranded nucleic acid refers to the position between the two nucleotides in the first strand complementary to those in the second strand between which nicking occurs.

A nucleic acid sequence (or region) is “upstream to” another nucleic acid sequence (or region) when the nucleic acid sequence is located 5′ to the other nucleic acid sequence. A nucleic acid sequence (or region) is “downstream to” another nucleic acid sequence (or region) when the nucleic acid sequence is located 3′ to the other nucleic acid sequence.

### B. Methods and Compositions for Exponential Amplification of Nucleic Acids

The present invention provides methods and compositions for exponential amplification of nucleic acids using nicking endonucleases. The following sections first provide a general description of the methods, and subsequently provide descriptions of two types of nucleic acid amplification methods, and compositions or kits for nucleic acid amplification.

1. General Description

In one aspect, the present invention provides a simple and fast method for exponential amplification of nucleic acids. It uses two or more linked amplification reactions (i.e., a tandem amplification system) catalyzed by the combination of a nicking agent (NA) and a DNA polymerase. Each amplification reaction is based on the ability of a NA to nick a double-stranded or partially double-stranded nucleic acid molecule that comprises the recognition sequence of the NA and the ability of a DNA polymerase to extend from the 3′ terminus at a nicking site (NS) of the NA.

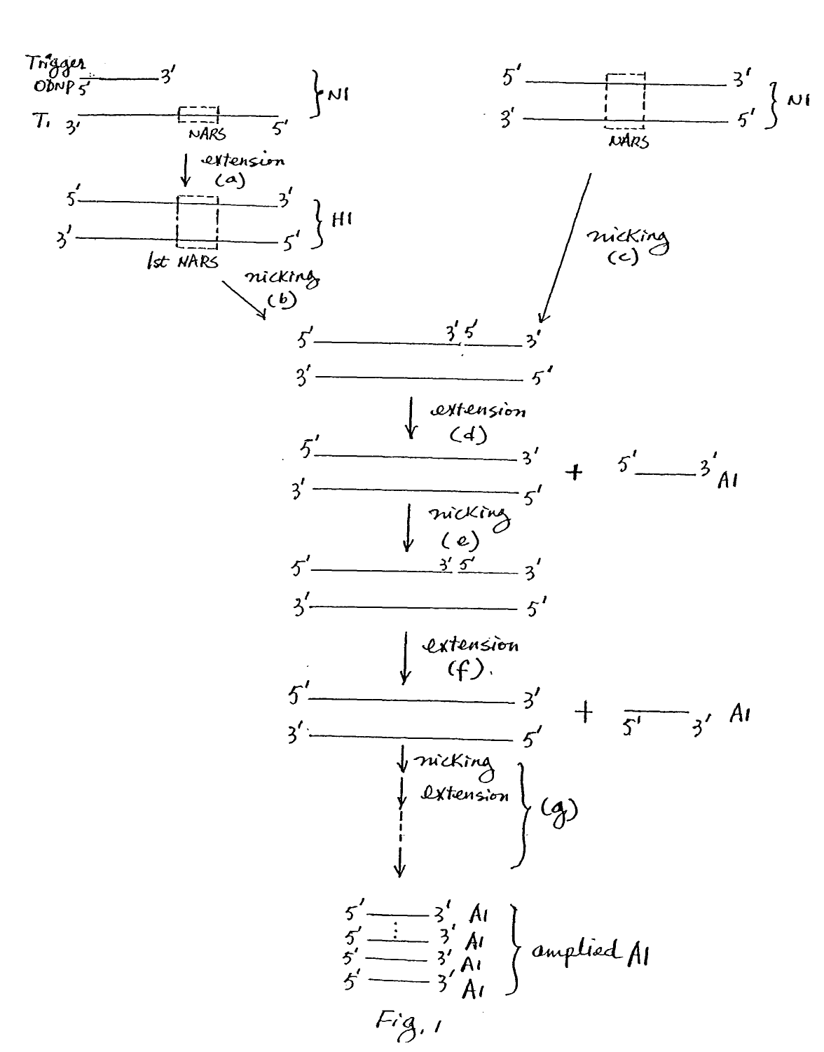

In the first amplification reaction (FIG. 1), a trigger ODNP is hybridized to a first template nucleic acid (T1) that comprises the sequence of one strand of a NARS (referred to as a “first NARS”) to form a completely or partially double-stranded nucleic acid molecule (“the initial nucleic acid molecule of the first amplification reaction (N1)”). The trigger ODNP either does not contain the other strand of the first NARS and hybridizes to a portion of T1 located 3′ to the strand of the first NARS in T1, or contains the other strand of the first NARS so that its hybridization to T1 forms a nucleic acid molecule comprising a double-stranded first NARS. If a portion of T1 at its 5′ terminus forms a 5′ overhang in N1, in the presence of a DNA polymerase (referred to as a “first DNA polymerase”), the trigger ODNP is extended using T1 as a template to form a hybrid (H1) that comprises the double-stranded first NARS (step (a) of FIG. 1). The resulting H1 may be nicked by a NA that recognizes the first NARS, producing a 3′ terminus and a 5′ terminus at the nicking site (step (b)). If the fragment containing the 5′ terminus at the nicking site (referred to as “A1”) is sufficiently short (e.g., less than 18 nucleotides in length), it will dissociate from the other portion of H1 under dissociative reaction conditions (e.g., at 60° C.). However, if this fragment (i.e., A1) does not readily dissociate, it may be displaced by the extension of the remaining fragment from its 3′ terminus at the NS in the presence of a first DNA polymerase that is 5′→3′ exonuclease deficient and has a strand displacement activity (step (d)). Strand displacement may also occur in the absence of strand displacement activity in the first DNA polymerase, if a strand displacement facilitator is present. Such extension recreates a new NS for the first NA that can be nicked again (“re-nicked”) as in the first NA (step (e)). The fragment containing the 5′ terminus at the new NS (i.e., a new A1) may again readily dissociate from the other portion of H1 or be displaced by extension from the 3′ terminus at the NS (step (f)). The nicking-extension cycles can be repeated multiple times (step (g)), resulting in the linear accumulation/amplification of the nucleic acid fragment A1.

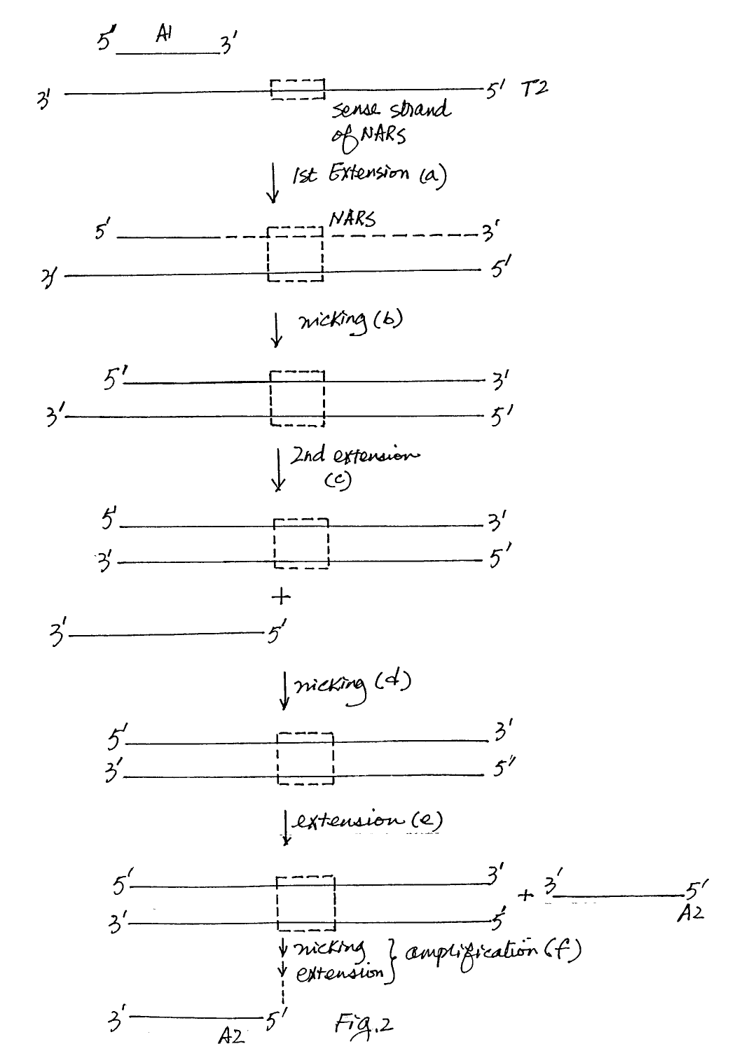

Exponential amplification of nucleic acid molecules may be performed by combining or linking the above-described first amplification reaction with a second amplification reaction via the amplified fragment A1 from the first amplification reaction. In the second amplification reaction (FIG. 2), A1 hybridizes to a portion of another single-stranded nucleic acid molecule (T2) that comprises a sequence of a sense strand of a second NARS. The resulting partially double-stranded nucleic acid molecule is referred to as “the initial nucleic acid molecule of the second amplification reaction (N2).” The portion of T2 to which A1 hybridizes is located 3′ to the sequence of the sense strand of the second NARS so that A1 functions as an initial primer for a primer extension reaction using T2 as a template. The extension from A1 produces a hybrid (H2) that comprises the double-stranded second NARS (step (a) of FIG. 2). In the presence of a second NA that recognizes the NARS, H2 is nicked, producing a 3′ terminus and a 5′ terminus at the nicking site (step (b)). If the fragment containing the 5′ terminus at the nicking site is sufficiently short (e.g., less than 18 nucleotides in length), it may dissociate from the other portion of H2 under certain reaction conditions (e.g., at 60° C.). However, if this fragment does not readily dissociate from the other portion of H2, it may be displaced by extension of the fragment having a 3′ terminus at the NS in the presence of a DNA polymerase (referred to as a “second DNA polymerase”) that is 5′→3′ exonuclease deficient and has a strand displacement activity (step (c)). Strand displacement may also occur in the absence of the strand displacement activity of the second DNA polymerase, but in the presence of a strand displacement facilitator. Such extension recreates a new NS for the second NA that can be nicked again (“re-nicked”) by the second NA (step (d)). The fragment containing the 5′ terminus at the new NS (referred to as “A2”) may again readily dissociate from the other portion of H2 or be displaced by extension from the 3′ terminus at the NS (step (e)). The nicking-extension cycles can be repeated multiple times (step (f)), resulting the exponential accumulation/amplification of the nucleic acid fragment A2. The amplified single-stranded strand nucleic acid fragment A2 is at least substantially complementary to A1. A2 may be completely complementary to A1 if A2 is of the same length as A1.

In one aspect, the present invention provides a method for amplifying a nucleic acid molecule (A2) comprising (a) providing a single-stranded nucleic acid molecule (A1); (b) providing a second single-stranded nucleic acid molecule (T2) comprising, from 5′ to 3′: (i) a sequence of the sense strand of a NARS, and (ii) a sequence that is at least substantially complementary to A1; and (c) amplifying A2 in he presence of T2, A1 a nicking agent that recognizes the NARS, a DNA polymerase, and one or more deoxynucleoside triphosphate(s), wherein A2 is at least substantially complementary to A1 and wherein A1, A2 or both are at most 25 nucleotides in length. Exemplary means by which A1 may be provided are described therein.

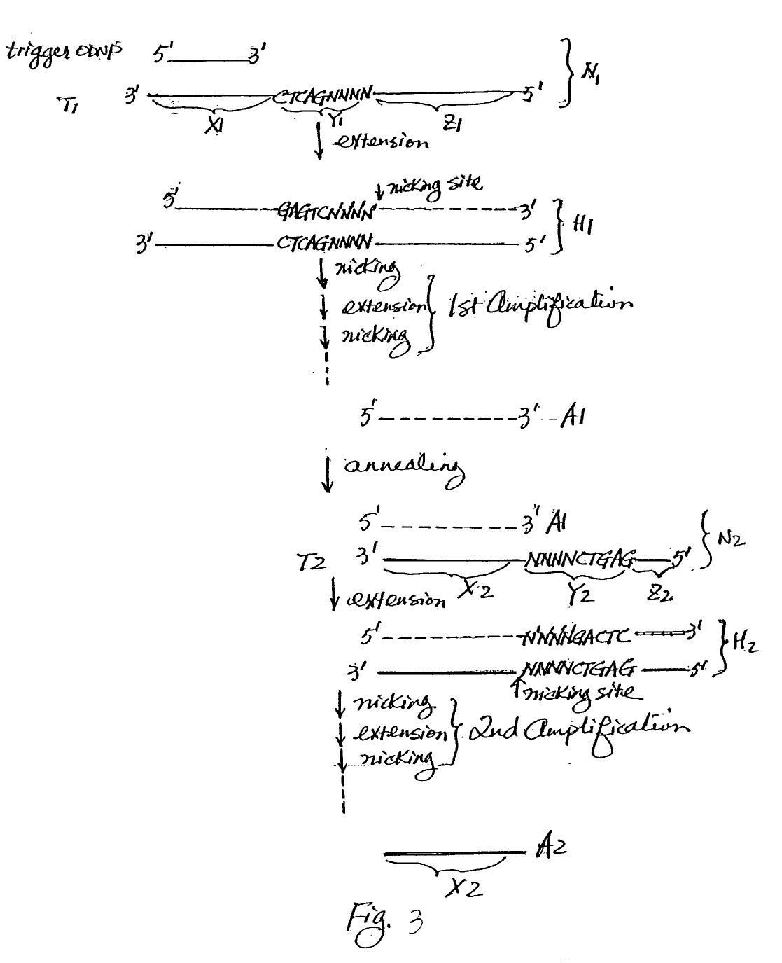

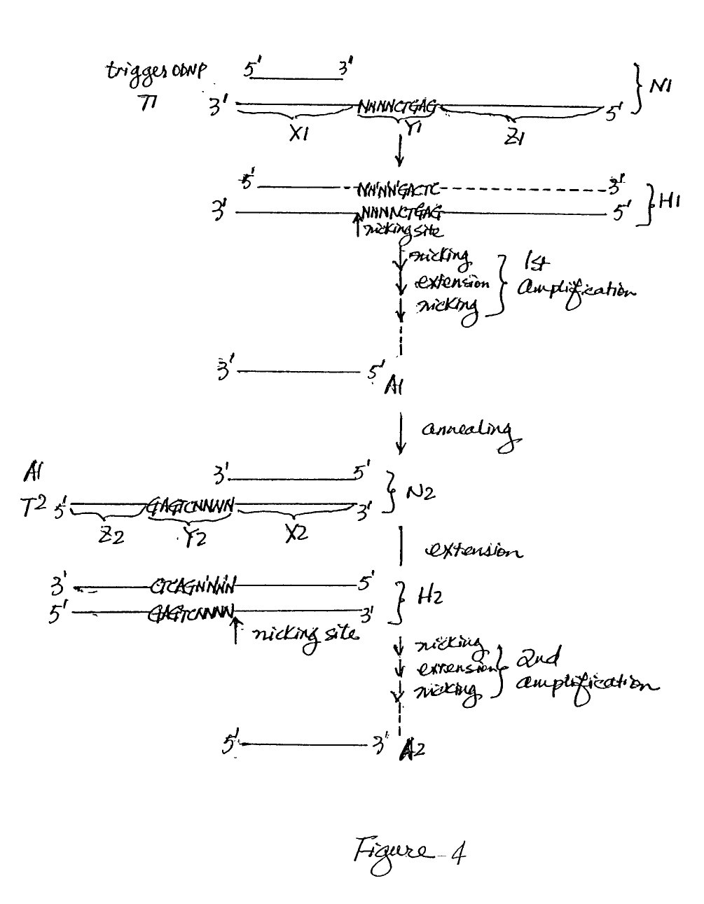

Although the exponential nucleic acid amplification method of the present invention requires that T2 comprise a sequence of a sense strand of a NARS, T1 may comprise a sequence of a sense strand or an antisense strand of a NARS. These two types of nucleic acid amplification reactions are illustrated in FIGS. 3 and 4 using the recognition sequence of N.BstNB I as an example for both the first and second NARSs. However, one of ordinary skill in the art appreciates that T1 and T2 may comprise the recognition sequences of other nicking agents.

The first type of nucleic acid amplification according to the present invention is where T1 comprises a sequence of an antisense strand of a first NARS. As shown in FIG. 3, for the first amplification reaction, the initial nucleic acid molecule N1 is a partially double-stranded nucleic acid molecule formed by annealing a trigger ODNP with T1 that has three regions: Regions X1, Y1 and Z1. Regions X1, Y1 and Z1 are defined as the region directly 3′ to the sequence of the antisense strand of the N.BstNB I recognition sequence, the region from the 3′ terminus of the sequence of the antisense strand of the recognition sequence of N.BstNB I to the nucleotide corresponding to the 3′ terminal nucleotide at the nicking site of N.BstNB I within the extension product of the trigger ODNP (i.e., 3′-CACAGNNNN-5′ where N can be A, T, G or C), and the region directly 5′ to Region Y1, respectively. The trigger ODNP is at least substantially complementary to Region X1 and functions as a primer for nucleic acid extension in the presence of a DNA polymerase. The extension product (H1) can be completely or partially double-stranded, depending on whether the 5′ terminal sequence of the trigger ODNP anneals to the 3′ terminal sequence of Region X1. Because H1 comprises the double-stranded N.BstNB I recognition sequence, it can be nicked by N.BstNB I. In certain embodiments, the 3′ terminus of T1 is blocked, such as by a phosphate group, so that the extension from this terminus is prevented. The nicked product comprising the sequence of the trigger ODNP may be extended again from its 3′ terminus at the nicking site by the DNA polymerase, which displaces the strand containing the 5′ terminus produced by N.BstNB I at the nicking site. The nicking-extension cycle is repeated multiple times, which accumulates the displaced strand (A1).

The product of the first amplification reaction A1 is then used as an initial primer for the second amplification reaction. It is annealed to Region X2 of T2, which also has two additional regions: Regions Y2 and Z2, to form an initial nucleic acid molecule N2 for the second amplification reaction. Region Y2 consists of a sequence of the sense strand of the recognition sequence of N.BstNB I and four nucleotides directly 3′ to the sequence (i.e., 3′-NNNNCTGAG-5′ where each of the Ns may be A, T, G, or C), whereas Regions X2 and Z2 refer to regions immediately next to the 3′ terminus and the 5′ terminus of Region Y2, respectively. The extension of A1 using T2 as a template provides an extension product (H2) that can be completely or partially double-stranded, depending on whether the 5′ terminal sequence of A1 anneals to the 3′ terminal sequence of Region X2. Because H2 comprises the double-stranded N.BstNB I recognition sequence, it can be nicked in the presence of N.BstNB I. The resulting 3′ terminus at the nicking site may be extended again by the DNA polymerase, which displaces Region X2. The nicking-extension cycle is repeated multiple times, resulting in the accumulation/amplification of a displaced strand A2 that contains the 5′ terminus at the nicking site. A2 is exactly identical to Region X2 if the 5′ terminal sequence of A1 anneals to the 3′ terminal sequence of Region X2. Otherwise, A2 and Region X2 is substantially complementary to each other as they have different lengths. The amplification of A2 is exponential because it is the final amplification product of two linked linear amplification reactions.

The second type of nucleic acid amplification according to the present invention is where T1 comprises a sequence of a sense strand of a first NARS. Preferably, the first NARS is identical to the second NARS. Using N.BstNB I as an exemplary NA whose sequence of the sense strand is present in both T1 and T2, this type of nucleic acid amplification is illustrated in FIG. 4.

As shown in FIG. 4, for the first amplification reaction, the initial nucleic acid molecule N1 is a partially double-stranded nucleic acid molecule formed by annealing a trigger ODNP with T1 having three regions: Regions X1, Y1 and Z1. Regions X1, Y1 and Z1 are defined as the region directly 3′ to the nicking site of the extension product of N1 (i.e., H1) by N.BstNB I, the region from the nicking site to the 5′ terminus of the sequence of the sense strand of the recognition sequence of N.BstNB I (i.e., 5′-GAGTCNNNN-3′ where N can be A, T, G or C), and the region directly 5′ to Region Y2, respectively. The trigger ODNP is at least substantially complementary to Region X1 or a portion thereof and functions as a primer for nucleic acid extension in the presence of a DNA polymerase. The extension product (H1) can be completely or partially double stranded, depending on whether the 5′ terminal sequence of the trigger ODNP anneals to the 3′ terminal sequence of Region X1. Because H1 comprises the double-stranded N.BstNB I recognition sequence, it can be nicked by N.BstNB I. In certain embodiments, the 3′ terminus of T1 is blocked, such as by a phosphate group, so that the extension from this terminus is prevented. The nicked product comprising the sequence of the sense strand of the recognition sequence of N.BstNB I may be extended again from its 3′ terminus at the nicking site by the DNA polymerase, which displaces the strand containing the 5′ terminus produced by N.BstNB I at the nicking site. The nicking-extension cycle is repeated multiple times, resulting in the accumulation of the displaced strand A1 containing the 5′ terminus of the nicking site.

The product of the first amplification reaction A1 is then used as an initial primer for the second amplification reaction. It is annealed to Region X2 of T2, (which contains two additional regions, i.e., Regions Y2 and Z2), to form an initial nucleic acid molecule N2 for the second amplification reaction. Region Y2 is similar to Region Y1 and has the sequence of the sense strand of the recognition sequence of N.BstNB I and four nucleotides located directly 3′ to the sequence of the sense strand of the N.BstNB I recognition sequence (i.e., 5′-GAGTCNNNN-3′ wherein N can be A, T, G or C). Regions X2 and Z2 refer to regions immediately next to the 3′ terminus and the 5′ terminus of Region Y2, respectively. The extension of A1 using T2 as a template provides an extension product (H2) that can be completely or partially double-stranded, depending on whether the 5′ terminal sequence of A1 anneals to the 3′ terminal sequence of Region X2. Because H2 comprises the double-stranded N.BstNB I recognition sequence, it can be nicked by N.BstNB I. The resulting 3′ terminus at the nicking site may be extended again by the DNA polymerase, which displaces Region X2. The nicking-extension cycle is repeated multiple times, resulting in accumulation/amplification of a displaced strand A2 that contains the 5′ terminus at the nicking site. The amplification of A2 is exponential because it is the final amplification product of two linked linear amplification reactions.

The present method is not limited to linking two nucleic acid amplification reactions together. In certain embodiments, a second amplification reaction may be further linked to a third amplification reaction. In other words, the nucleic acid molecule A2 amplified during the second amplification reaction may anneal to a portion of another nucleic acid molecule “T3” that comprises the sequence of one strand of a NARS (referred to as a “third NARS”) to trigger the amplification of a nucleic acid molecule “A3” in a third amplification reaction. Additional amplification reactions may be added to the chain. For example, A3 may in turn anneal to a portion of another nucleic acid molecule “T4” also comprising one strand of a NARS (referred to as a “fourth NARS”) and initiate the amplification of a nucleic acid molecule “A4” in a fourth amplification reaction. Because each subsequent amplification reaction results in a linear amplification of the amplified fragment from its previous amplification reaction, the greater number of the amplification reactions in an amplification system, the higher level of amplification, provided that the other components of the system (e.g., template nucleic acid molecules, NAs, and DNA polymerases) do not limit the amplification rate or level.

a. Nicking Agents

As described above, the exponential nucleic acid amplification method of the present invention links two or more nucleic acid amplification reactions together and each amplification reaction is performed in the presence of a NA. The NA for one amplification reaction may be different from that for another amplification reaction. In one embodiment, the NAs for different amplification reactions are identical to each other, so that only one NA is required for exponential amplification of a nucleic acid molecule. In another embodiment, two different NAs, e.g., two NAs recognizing different NARSs, are employed.

Any enzyme that recognizes a specific nucleotide sequence and cleaves only one strand of a fully or partially double-stranded nucleic acid that comprises the sequence may be used as a nicking agent in the present invention. Such an enzyme can be a NE that recognizes a specific sequence that consists of native nucleotides or a RE that recognizes a hemimodified recognition sequence.

A nicking endonuclease may or may not have a nicking site that overlaps with its recognition sequence. An exemplary NE that nicks outside its recognition sequence is N.BstNB I, which recognizes a unique nucleic acid sequence composed of 5′-GAGTC-3′, but nicks four nucleotides beyond the 3′ terminus of the recognition sequence. The recognition sequence and the nicking site of N.BstNB I are shown below with “▾” to indicate the cleavage site where the letter N denotes any nucleotide:

▾
5′-GAGTCNNNNN-3′
3′-CTCAGNNNNN-5′

N.BstNB I may be prepared and isolated as described in U.S. Pat. No. 6,191,267. Buffers and conditions for using this nicking endonuclease are also described in the '267 patent. An additional exemplary NE that nicks outside its recognition sequence is N.AlwI, which recognizes the following double-stranded recognition sequence:

▾
5′-GGATCNNNNN-3′
3′-CCTAGNNNNN-5′

The nicking site of N.AlwI is also indicated by the symbol “▾”. Both NEs are available from New England Biolabs (NEB). N.AlwI may also be prepared by mutating a type IIs RE AlwI as described in Xu et al. (Proc. Natl. Acad. Sci. USA 98:12990-5, 2001).

Exemplary NEs that nick within their NERSs include N.BbvCl-a and N.BbvCl-b. The recognition sequences for the two NEs and the NSs (indicated by the symbol “▾”) are shown as follows:

| | |
|---|---|
| N.BbvCl-a | |
| | ▾ |
| | 5′-CCTCAGC-3′ |
| | 3′GGAGTCG 5′ |
| N.BbvCl-b | |
| | ▾ |
| | 5′-GCTGAGG-3′ |
| | 3′-CGACTCC-5′ |

Both NEs are available from NEB.

Additional exemplary nicking endonucleases include, without limitation, N.BstSE I (Abdurashitov et al., Mol. Biol. (Mosk) 30: 1261-7, 1996), an engineered EcoR V (Stahl et al., Proc. Natl. Acad. Sci. USA 93: 6175-80, 1996), an engineered Fok I (Kim et al., Gene 203: 43-49, 1997), endonuclease V from Thermotoga maritima (Huang et al., Biochem. 40: 8738-48, 2001), Cvi Nickases (e.g., CviNY2A, CviNYSI, Megabase Research Porducts, Lincoln, Nebr.) (Zhang et al., Virology 240: 366-75, 1998; Nelson et al., Biol. Chem. 379: 423-8, 1998; Xia et al., Nucleic Acids Res. 16: 9477-87, 1988), and an engineered Mly I (i.e., N.Mly I) (Besnier and Kong, EMBO Reports 2: 782-6, 2001). Additional NEs may be obtained by engineering other restriction endonuclease, especially type IIs restriction endonucleases, using methods similar to those for engineering EcoR V, AlwI, Fok I and/or Mly I.

A RE useful as a nicking agent can be any RE that nicks a double-stranded nucleic acid at its hemimodified recognition sequences. Exemplary REs that nick their double-stranded hemimodified recognition sequences include, but are not limited to Ava I, Bsl I, BsmA I, BsoB I, Bsr I, BstN I, BstO I, Fnu4H I, Hinc II, Hind II and Nci I. Additional REs that nick a hemimodified recognition sequence may be screened by the strand protection assays described in U.S. Pat. No. 5,631,147.

Certain nicking agents require only the presence of the sense strand of a double-stranded recognition sequence in an at least partially double-stranded substrate nucleic acid for their nicking activities. For instance, N.BstNB I is active in nicking a substrate nucleic acid that comprises, in one strand, the sequence of the sense strand of its recognition sequence “5′-GAGTC-3′” of which one or more nucleotides do not form conventional base pairs (e.g., G:C, A:T, or A:U) with the other strand of the substrate nucleic acid. The nicking activities of N.BstNB I decreases with the increase of the number of the nucleotides in the sense strand of its recognition sequence that do not form conventional base pairs with any nucleotides in the other strand of the substrate nucleic acid.

In certain embodiments, a nicking agent may recognize a nucleotide sequence in a DNA-RNA duplex and nicks in one strand of the duplex. In certain other embodiments, a nicking agent may recognize a nucleotide sequence in a double-stranded RNA and nicks in on strand of the RNA.

b. DNA Polymerases

As described above, the exponential nucleic acid amplification method of the present invention links two or more nucleic acid amplification reactions together and each amplification reaction is performed in the presence of a DNA polymerase. The DNA polymerase for one amplification reaction may be different from that for another amplification reaction. In one embodiment, the DNA polymerases for different amplification reactions are identical to each other, so that only one DNA polymerase is required for exponential amplification of a nucleic acid molecule.

The DNA polymerase useful in the present invention may be any DNA polymerase that is 5′→3′ exonuclease deficient but has a strand displacement activity. Such DNA polymerases include, but are not limited to, exo⁻ Deep Vent, exo⁻ Bst, exo⁻ Pfu, and exo⁻ Bca. Additional DNA polymerase useful in the present invention may be screened for or created by the methods described in U.S. Pat. No. 5,631,147, incorporated herein by reference in its entirety. The strand displacement activity may be further enhanced by the presence of a strand displacement facilitator as described below.

Alternatively, in certain embodiments, a DNA polymerase that does not have a strand displacement activity may be used. Such DNA polymerases include, but are not limited to, exo⁻ Vent, Taq, the Klenow fragment of DNA polymerase I, T5 DNA polymerase, and Phi29 DNA polymerase. In certain embodiments, the use of these DNA polymerases requires the presence of a strand displacement facilitator. A “strand displacement facilitator” is any compound or composition that facilitates strand displacement during nucleic acid extensions from a 3′ terminus at a nicking site catalyzed by a DNA polymerase. Exemplary strand displacement facilitators useful in the present invention include, but are not limited to, BMRF1 polymerase accessory subunit (Tsurumi et al., J. Virology 67: 7648-53, 1993), adenovirus DNA-binding protein (Zijderveld and van der Vliet, J. Virology 68: 1158-64, 1994), herpes simplex viral protein ICP8 (Boehmer and Lehman, J. Virology 67: 711-5, 1993; Skaliter and Lehman, Proc. Natl. Acad. Sci. USA 91: 10665-9, 1994), single-stranded DNA binding protein (Rigler and Romano, J. Biol Chem. 270: 8910-9, 1995), phage T4 gene 32 protein (Villemain and Giedroc, Biochemistry 35: 14395-4404, 1996), calf thymus helicase (Siegel et at., J. Biol Chem. 267: 13629-35, 1992) and trehalose. In one embodiment, trehalose is present in the amplification reaction mixture.

Additional exemplary DNA polymerases useful in the present invention include, but are not limited to, phage M2 DNA polymerase (Matsumoto et al., Gene 84: 247, 1989), phage PhiPRD1 DNA polymerase (Jung et al., Proc. Natl. Acad. Sci. USA 84: 8287, 1987), T5 DNA polymerase (Chatterjee et al., Gene 97: 13-19, 1991), Sequenase (U.S. Biochemicals), PRD1 DNA polymerase (Zhu and Ito, Biochim. Biophys. Acta. 1219: 267-76, 1994), 9° Nm™ DNA polymerase (New England Biolabs) (Southworth et al., Proc. Natl. Acad. Sci. 93: 5281-5, 1996; Rodriquez et al., J. Mol. Biol. 302: 447-62, 2000), and T4 DNA polymerase holoenzyme (Kaboord and Benkovic, Curr. Biol. 5: 149-57, 1995).

Alternatively, a DNA polymerase that has a 5′→3′ exonuclease activity may be used. For instance, such a DNA polymerase may be useful for amplifying short nucleic acid fragments that automatically dissociate from the template nucleic acid after nicking.

In certain embodiments where a nicking agent nicks in the DNA strand of a RNA-DNA duplex, a RNA-dependent DNA polymerase may be used. In other embodiments where a nicking agent nicks in the RNA strand of a RNA-DNA duplex, a DNA-dependent DNA polymerase that extends from a DNA primer, such as Avian Myeloblastosis virus reverse transcriptase (Promega) may be used. In both instances, a target mRNA need not be reverse transcribed into cDNA and may be directly mixed with a template nucleic acid molecule that is at least substantially complementary to the target mRNA.

c. Reaction Conditions

The exponential nucleic acid amplification method of the present invention links two or more nucleic acid amplification reactions where each utilizes nicking and primer extension reactions in achieving amplification. According to the methods of the present invention, in each amplification reaction, a DNA polymerase may be mixed with nucleic acid molecules (e.g., template nucleic acid molecules) before, after, or at the same time as, a NA is mixed with the template nucleic acid. Preferably, the nicking-extension reaction buffer is optimized to be suitable for both the NA and the DNA polymerase. For instance, if N.BstNB I is the NA and exo⁻ Vent is the DNA polymerase, the nicking-extension buffer can be 0.5×N.BstNB I buffer and 1×DNA polymerase Buffer. Exemplary 1×N.BstNB I buffer may be 10 mM Tris-HCl, 10 mM MgCl₂, 150 mM KCl, and 1 mM dithiothreitol (pH 7.5 at 25° C). Exemplary 1×DNA polymerase buffer may be 10 mM KCl, 20 mM Tris-HCl (pH 8.8 at 25° C.), 10 mM (NH₄)₂SO₄, 2 mM MgSO₄, and 0.1% Triton X-100. One of ordinary skill in the art is readily able to find a reaction buffer for a NA and a DNA polymerase.

In addition, in certain embodiments where a DNA polymerase is dissociative (i.e., the DNA polymerase is relatively easy to dissociate from a template nucleic acid, such as Vent DNA polymerase), the ratio of a NA to a DNA polymerase in a reaction mixture may also be optimized for maximum amplification of full-length nucleic acid molecules. As used herein, a “full-length” nucleic acid molecule refers to an amplified nucleic acid molecule that contains the sequence complementary to the 5′ terminal sequence of its template. In other words, a full-length nucleic acid molecule is an amplification product of a complete gene extension reaction. In a reaction mixture where the amount of a NA is excessive with respect to that of a DNA polymerase, partial amplification products may be produced. The production of partial amplification products may be due to excessive nicking of partially amplified nucleic acid molecules by the NA and subsequent dissociation of these molecules from their templates. Such dissociation prevents the partially amplified nucleic acid molecules from being further extended.

Because different NAs or different DNA polymerases may have different nicking or primer extension activities, the ratio of a particular NA to a specific dissociative DNA polymerase that is optimal to maximum amplification of full-length nucleic acids will vary depending on the identities of the specific NA and DNA polymerase. However, for a given combination of a particular NA and a specific DNA polymerase, the ratio may be optimized by carrying out exponential nucleic acid amplification reactions in reaction mixtures having different NA to DNA polymerase ratios and characterizing amplification products thereof using techniques known in the art (e.g., by liquid chromatography or mass spectrometry). The ratio that allows for maximum production of full-length nucleic acid molecules may be used in future amplification reactions.

It is noteworthy that although partial amplification of nucleic acid molecules may occur during both the first and the second amplification reactions, partial amplification during the first amplification reaction usually does not significantly affect the overall nucleic acid amplification level or rate. Because the nucleic acid molecules amplified during the first amplification reaction are used as an initial amplification primer for the second amplification reaction, they are sufficient for their intended use if they are long enough to allow for their specific annealing to their templates. Besides using the optimal ratio of a NA to a dissociative DNA polymerase for full-length nucleic acid amplification, alternatively, the amount of partial amplification products may be eliminated or reduced by inactivating the NA but not the DNA polymerase (e.g., by heat inactivation) after amplification reactions have proceeded for a period of time and allowing each gene extension reaction to proceed to its completion.

In certain preferred embodiments, nicking and extension reactions of the present invention are performed under isothermal conditions. As used herein, “isothermally” and “isothermal conditions” refer to a set of reaction conditions where the temperature of the reaction is kept essentially constant (i.e., at the same temperature or within the same narrow temperature range wherein the difference between an upper temperature and a lower temperature is no more than 20° C.) during the course of the amplification. An advantage of the amplification method of the present invention is that there is no need to cycle the temperature between an upper temperature and a lower temperature. Both the nicking and the extension reaction will work at the same temperature or within the same narrow temperature range. If the equipment used to maintain a temperature allows the temperature of the reaction mixture to vary by a few degrees, such a fluctuation is not detrimental to the amplification reaction. Exemplary temperatures for isothermal amplification include, but are not limited to, any temperature between 50° C. to 70° C. or the temperature range between 50° C. to 70° C., 55° C. to 70° C., 60° C. to 70° C., 65° C. to 70° C., 50° C. to 55° C., 50° C. to 60° C., or 50° C. to 65° C. Many NAs and DNA polymerases are active at the above exemplary temperatures or within the above exemplary temperature ranges. For instance, both the nicking reaction using N.BstNB I (New England Biolabs) and the extension reaction using exo⁻ Bst polymerases (BioRad) may be carried out at about 55° C. Other polymerases that are active between about 50° C. and 70° C. include, but are not limited to, exo⁻ Vent (New England Biolabs), exo⁻ Deep Vent (New England Biolabs), exo⁻ Pfu (Strategene), exo⁻ Bca (Panvera) and Sequencing Grade Taq (Promega).

When a restriction endonuclease is used as a nicking agent, a modified deoxyribonucleoside triphosphate is needed to produce a hemimodified restriction endonuclease recognition sequence. Any modified deoxyribonucleoside triphosphate that contributes to the inhibition of cleavage of one strand of a double-stranded nucleic acid comprising the modified deoxyribonucleoside triphosphate in a restriction endonuclease recognition sequence may be used. Exemplary modified deoxyribonucleoside triphosphates include, but are not limited to, 2′-deoxycytidine 5′-O-(1-thiotriphosphate) [i.e., dCTP(.alpha.S)], 2′-deoxyguanosine 5′-O-(1-thiotriphosphate), thymidine-5′-O-(1-thiotriphosphate), 2′-deoxycytidine 5′-O(1-thiotriphosphate), 2′-deoxyuridine 5′-triphosphate, 5-methyldeoxycytidine 5′-triphosphate, and 7-deaza-2′-deoxyguanosine 5′-triphosphate.

d. Initial Nucleic Acids (N1s)

As discussed above, the initial nucleic acid for the first nucleic acid amplification (i.e., N1) may be provided by annealing a trigger ODNP with a template nucleic acid molecule T1. Because the trigger ODNP functions as a primer for primer extension using T1 as a template, it must be substantially complementary to a portion of T1 and also have a 3′ terminus, from which primer extension occurs.

In certain embodiments, the trigger ODNP is derived from a nucleic acid molecule. The 3′ terminus of the trigger may be produced by various methods known in the art. For instance, the 3′ terminus of a trigger ODNP may be provided by digesting a nucleic acid fragment having a restriction endonuclease recognition sequence (RERS) using a restriction endonuclease that recognizes the RERS (e.g., a type IIs restriction endonuclease). The RERS in the nucleic acid fragment may be naturally occurring or may be incorporated into the fragment by using a primer that comprises one strand of the RERS. Alternatively, the 3′ terminus of a trigger ODNP may be produced by nicking a nucleic acid fragment having a NARS with a NA that recognizes the NARS. The NARS may also be naturally occurring or may be incorporated into the fragment by using a primer that comprises one strand of the NARS. In addition, the 3′ terminus of a trigger ODNP may be created by oligonucleotide-directed cleavage according to Szybalski (U.S. Pat. No. 4,935,357) or by base-specific chemical cleavage according to Maxam-Gilbert (Proc. Natl. Acad. Sci. USA 74:560-4, 1977). In certain embodiments, the 3′ terminus of a trigger ODNP may be provided by cleaving a nucleic acid molecule with DNase I or other non-specific nucleases or by shearing a nucleic acid molecule. In situations where the cleavage product is a double-stranded nucleic acid, a trigger ODNP may be obtained by denaturing the double-stranded nucleic acid.

The nucleic acid molecule from which the trigger ODNP is derived may be naturally occurring or synthetic. It may be RNA or DNA, single-stranded or double-stranded. Such nucleic acid molecules include genomic DNA, cDNA or its derivates, such as randomly primed or specifically primed amplification products. The trigger ODNP itself may be a single-stranded DNA molecule or a single-stranded RNA molecule. The trigger ODNP or the nucleic acid molecule from which the trigger ODNP is derived may or may not be immobilized to a solid support.

Likewise, T1 may also be derived from another nucleic acid molecule by enzymatic, chemical, or mechanic cleavages. Enzymatic cleavages may be accomplished, for example, by digesting the nucleic acid molecule with a restriction endonuclease that recognizes a specific sequence within the nucleic acid molecule. Alternatively, enzymatic cleavages may be accomplished by nicking the nucleic acid molecule with a nicking agent that recognizes a specific sequence within the nucleic acid molecule. Enzymatic cleavages may also be oligonucleotide-directed cleavages according to Szybalski (U.S. Pat. No. 4,935,357). Chemical and mechanic cleavages may be accomplished by any method known in the art suitable for cleaving nucleic acid molecules such as shearing. In situations where the cleavage product is a double-stranded nucleic acid molecule, a T1 molecule may be obtained by denaturing the double-stranded nucleic acid molecule.

As noted above, T1 contains a sequence of one strand of a NARS. The NARS may be present in the nucleic acid molecule from which T1 is derived. Alternatively, it may be incorporated into T1, for example, by using an ODNP comprising a sequence of one strand of the NARS.

Similar to trigger ONDPs, T1 molecules may be derived from various nucleic acid molecules. These nucleic acid molecules include naturally occurring nucleic acids and synthetic nucleic acids, either of which may be double-stranded or single-stranded nucleic acid molecules, and may be DNAs (such as genomic DNA and cDNA) or RNAs.

In certain embodiments, a T1 molecule comprises or consists essentially of, from 3′ to 5′: a first sequence that is at most 100 nucleotides in length; a sequence of one strand of a double-stranded nicking agent recognition sequence; and a second sequence that is at most 100 nucleotides in length. In some embodiments, a T1 molecule is at most 200, 150, 100, 80, 60, 50, 40, 30, 25, 20, 18, 16, 14, 12, or 10 nucleotides in length. The first sequence, the second sequence, or both, in certain embodiments, may be at most 100, 80, 60, 50, 40, 30, 25, 20, 18, 16, 14, 12, 10, 9, 8, 7, 6, 5, or 4 nucleotides in length.

In certain embodiments where (1) a T1 comprises a sequence of the sense strand of a nicking agent recognition sequence and (2) a trigger ODNP is complementary to a portion of the T1 molecule that flanks the sequence of the sense strand of the nicking agent recognition sequence, there may be mismatches between one or more nucleotides within the sense strand of the nicking agent recognition sequence in the T1 and the corresponding nucleotides in the trigger ODNP. In other words, one or more nucleotides within the sense strand of the nicking agent recognition sequence in the T1 may not form conventional base pair(s) with any nucleotides in the trigger ODNP. Because certain nicking agents (e.g., N.BstNB I) are capable of nicking a substrate that comprises only the sense strand of their double-stranded recognition sequences, the initial nucleic acid (N1) formed by annealing the trigger ODNP to the T1 may be used as a template to amplify a single-stranded nucleic acid (A1) in the presence of a nicking agent that recognizes the sense strand of the recognition sequence in the T1 molecule. The detailed descriptions for the use of a nicking agent to amplify a single-stranded nucleic acid using a template nucleic acid that comprises only the sequence of the sense strand, not the intact antisense strand, of a double-stranded nicking agent recognition sequence are provided in the U.S. application entitled to “Amplification of Nucleic Acid Fragments Using Nicking Agents.”

Alternative to the embodiments where a trigger ODNP, T1, or both are derived from a nucleic acid molecule, the present invention also includes embodiments where the trigger ODNP, T1, or both are synthetic nucleic acid molecules. Any method known in the art for oligonucleotide synthesis may be used to synthesize trigger ODNP and/or T1. For instance, trigger ODNP and/or T1 may be synthesized by the solid phase oligonucleotide synthesis methods disclosed in U.S. Pat. Nos. 6,166,198, 6,043,353, 6,040,439, and 5,945,524 (incorporated herein in their entireties by reference). Briefly, solid phase oligonucleotide synthesis can be performed by sequentially linking 5′ blocked nucleotides to a nascent oligonucleotide attached to a resin, followed by oxidizing and unblocking to form phosphate diester linkages. In addition, the trigger ODNP and/or T1 may be purchased from companies that synthesize customer-designed oligonucleotides.

T1 may be immobilized to a solid support in certain embodiments. Preferably, T1 is immobilized via its 5′ terminus. In other embodiments, T1 may not be immobilized to a solid support.

In certain embodiments, the initial nucleic acid molecule of the first amplification reaction (i.e., N1) may be provided other than by annealing a trigger ODNP with a template nucleic acid molecule T1. For instance, N1 may be a completely or partially double-stranded nucleic acid molecule comprising a double-stranded NARS, which can be readily nicked by a NA that recognizes the NARS (step (c) of FIG. 1) without any initial primer extension reaction (e.g., step (a) of FIG. 1). In such a case, each strand of the N1 molecule comprises a sequence of one strand of a NARS. Thus, either strand may be regarded as a T1 molecule with its complementary strand as a trigger ODNP. A double-stranded N1 molecule may be, for example, a digestion product of a nucleic acid comprising a NARS. The sequence of NARS in N1 may be originated or derived from another nucleotide sequence, or incorporated into N1 by an oligonucleotide primer comprising the sequence of one strand of the NARS or during the chemical synthesis of T1.

N1 may be a partially double-stranded nucleic acid molecule comprising either a double-stranded NARS or only one strand of a NARS. For instance, N1 may be a nicked product of a nucleic acid molecule comprising two NARSs or a nicking digestion product of a nucleic acid molecule comprising both a NARS and a RERS.

In certain embodiments, N1 may be immobilized to a solid support. In other embodiments, N1 may not be immobilized to a solid support.

e. T2 Molecules

Similar to T1, T2 may also be derived from another nucleic acid molecule by enzymatic, chemical or mechanic cleavages within the other nucleic acid molecule as described above, or by nucleic acid amplification using the other nucleic acid molecule as a template. The other nucleic acid molecule from which T2 is derived may be naturally occurring nucleic or synthetic, double-stranded or single-stranded nucleic acid, DNA (such as genomic DNA and cDNA) or RNA. In one embodiment T2 is chemically synthesized.

As described above, T2 contains a sequence of a sense strand of a NARS. The NARS may be present in the nucleic acid molecule from which T2 is derived. Alternatively, it may be incorporated into T2, for example, by using an ODNP comprising a sequence of one strand of the NARS.

The number of T2 molecules in an amplification reaction mixture is typically more than that of T1 molecules. The preference for a greater number of T2 molecules than T1 molecules is due to the fact that T2 molecules are used as annealing partners for the single-stranded nucleic acid molecules (i.e., A1) amplified using T1 molecules as templates. In other words, during the first amplification reaction, each T1 molecule is used as a template to produce multiple copies of A1. Thus, for each of the T1 molecules, multiple T2 molecules are preferably present to provide annealing partners for the multiple A1 molecules amplified using a single T1 molecule as a template.

In certain embodiments, a T2 molecule comprises or consists essentially of, from 3′ to 5′: a first sequence that is at most 100 nucleotides in length; a sequence of the sense strand of a double-stranded nicking agent recognition sequence; and a second sequence that is at most 100 nucleotides in length. In some embodiments, a T2 molecule is at most 200, 150, 100, 80, 60, 50, 40, 30, 25, 20, 18, 16, 14, 12, or 10 nucleotides in length. The first sequence, the second sequence, or both, in certain embodiments, may be at most 100, 80, 60, 50, 40, 30, 25, 20, 18, 16, 14, 12, 10, 9, 8, 7, 6, 5, or 4 nucleotides in length.

A T2 molecule may be immobilized to a solid support, preferably at its 5′ terminus. There may be a linker between the solid phase to which the T2 molecule is attached and the 5′ or 3′ terminus of the primer. In addition, multiple T2 molecules may be immobilized to a single solid phase to produce an array of T2 molecules. The multiple T2 molecules may have identical sequences at discrete locations of the array. Alternatively, they may have different sequences that are at least substantially complementary to various A1 molecules at distinct locations of the array. Such an array may be used to amplify multiple single-stranded nucleic acid molecules with different sequences. In certain embodiments, the amplification reactions performed at different locations of an array are physically separated, such as in microwells of a plate, so that the amplification products at different location are not mixed with each other and may be characterized individually.

2. Nucleic Acids, Compositions or Kits for Nucleic Acid Amplification

In one aspect, the present invention provides compositions and kits for exponential amplification of nucleic acids. Such compositions generally comprise a combination of a first at least partially double-stranded nucleic acid molecule (N1 or H1) and a second at least partially double-stranded nucleic acid molecule (N2 or H2) designed to function in the first or the second type of nucleic acid amplification described above. For instance, for the first type of nucleic acid amplification, the composition may comprise (1) a first at least partially double-stranded nucleic acid molecule (N1 or H1) of which one strand comprises a sequence of the antisense strand of a first NARS, and (2) a second nucleic acid (N2 or H2) that comprises, from 5′ to 3′: (i) a sequence of the sense strand of a second NARS, and (ii) a sequence that is at least substantially identical to a sequence located 5′ to the sequence of the antisense strand of the first NARS in the first nucleic acid molecule. For the second type of nucleic acid amplification, the composition may comprise (1) a first at least partially double-stranded nucleic acid molecule (N1 or H1) of which one strand comprises a sequence of a sense strand of a first NARS, and (2) a second at least partially double-stranded nucleic acid molecule (N2 or H2) of which one strand comprises, from 5′ to 3′: (i) a sequence of the sense strand of a second NARS, and (ii) a sequence that is at least substantially complementary to a sequence located 3′ to the sequence of the sense strand of the first NARS in the first nucleic acid molecule. In certain embodiments, for both types of nucleic acid amplification, the first NARS is identical to the second NARS.

The kit of the present invention may comprise one of the above compositions. Alternatively, the kit may comprises a combination of single-stranded nucleic acid molecules T1 and T2 designed to function in either the first or the second type of nucleic acid amplification described above. For instance, for the first type of nucleic acid amplification, the composition may comprise a T1 that comprises a sequence of the antisense strand of a first NARS, and a T2 that comprises, from 5′ to 3′: (i) a sequence of the sense strand of a second NARS, and (ii) a sequence that is at least substantially identical to a sequence located 5′ to the sequence of the antisense strand of the first NARS in the T1. For the second type of nucleic acid amplification, the composition may comprise a T1 that comprises a sequence of the sense strand of a first NARS, and a T2 that comprises, from 5′ to 3′: (i) a sequence of the sense strand of a second NARS, and (ii) a sequence that is at least substantially complementary to a sequence located 3′ to the sequence of the sense strand of the first NARS in the first nucleic acid molecule. In certain embodiments, for both types of nucleic acid amplification, the first NARS is identical to the second NARS.

In addition to the above-described nucleic acid molecules, the kits (or compositions) of the present invention may further comprise at least one, two, several, or each of the following components: (1) a trigger ODNP that is capable of specific annealing to the sequence of T1 3′ to the sequence of one strand of the NARS in T1; (2) a nicking agent (e.g., a NE or a RE) that recognizes the NARS of which the sequence of one strand is present in T1, T2 or both; (3) a buffer for nicking agent (2); (4) a DNA polymerase useful for primer extension; (5) a buffer for DNA polymerase (4); (6) deoxynucleoside triphosphates; (7) a modified deoxynucleoside triphosphate; (8) a control T1, T2 and/or trigger ODNP; and (9) a strand displacement facilitator (e.g., trehalose). Detailed descriptions of many of the above components are provided above.

In certain embodiments, the composition of the present invention does not contain a buffer specific to a NA or a buffer specific to a DNA polymerase. Instead, it contains a buffer suitable for both the nicking agent and the DNA polymerase. For instance, if N.BstNB I is the nicking agent and exo⁻ Vent is the DNA polymerase, the nicking-extension buffer can be 0.5×N.BstNB I buffer and 1×exo⁻ Vent Buffer.

The compositions of the present invention may be made by simply mixing their components or by performing reactions that results in the formation of the compositions. The kits of the present invention may be prepared by mixing some of their components or keep each of them in an individual container.

### C. Diagnostic Uses of Nucleic Acid Amplification Methods and Compositions

As described in detail herein above, the present invention provides methods and compositions for exponential amplification of nucleic acids. These methods and compositions may find utility in a wide variety of applications where it is desirable to rapidly amplify a nucleic acid molecule. Such rapid amplification may be especially desirable in diagnostic applications, such as where it is desirable to quickly detect the presence of a pathogen (e.g., bacteria, viruses, fungi, parasites) in a biological sample. The following sections describe various exemplary embodiments specifically applicable for diagnostic uses.

1. Overview

The present invention is useful for detecting a target nucleic acid molecule in a biological sample. The target nucleic acid includes a nucleic acid molecule that is derived or originates from a pathogenic organism. Depending on the presence or absence of the target nucleic acid in the sample, an amplification product may or may not be detected in an amplification system that is designed to use the target nucleic acid or its portion as a template. The target nucleic acid or its portion is first incorporated into an initial nucleic acid molecule (N1) to be used as a template in a first amplification reaction. The initial nucleic acid molecule also comprises at least one strand of a first NARS and thus triggers the first amplification reaction in the presence of a DNA polymerase and a NA that recognizes the first NARS. The product (A1) from the first amplification reaction then anneals to another template nucleic acid molecule (T2). T2 comprises a sequence of the sense strand of a second NARS and thus initiates a second amplification reaction in the presence of the DNA polymerase and a NA that recognizes the second NARS. The determination of the presence or absence of the product (A1) of the first amplification reaction and/or the product (A2) of the second amplification reaction indicates the presence or absence of the target nucleic acid in the biological sample.

2. Initial Nucleic Acid Molecules (N1s)

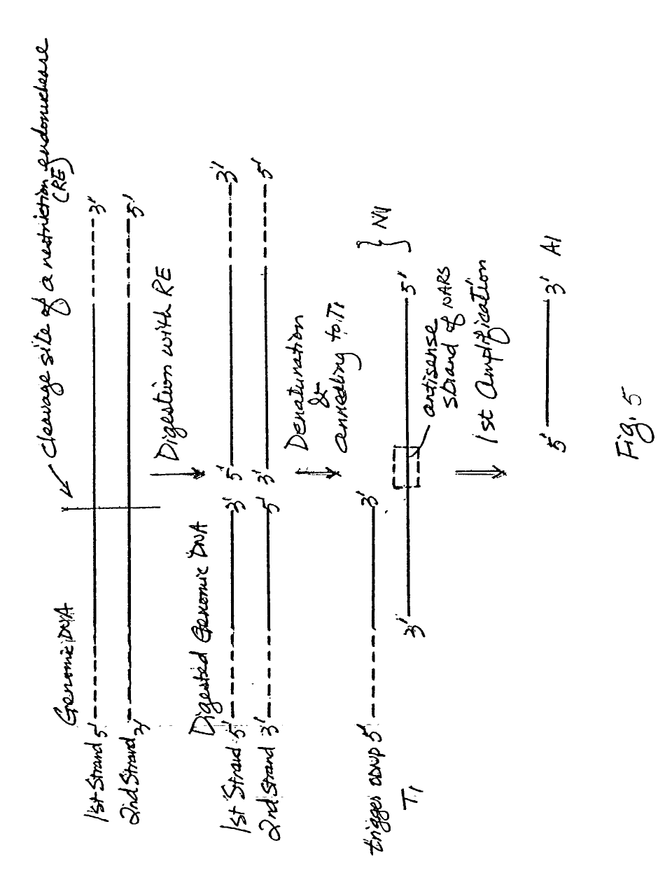

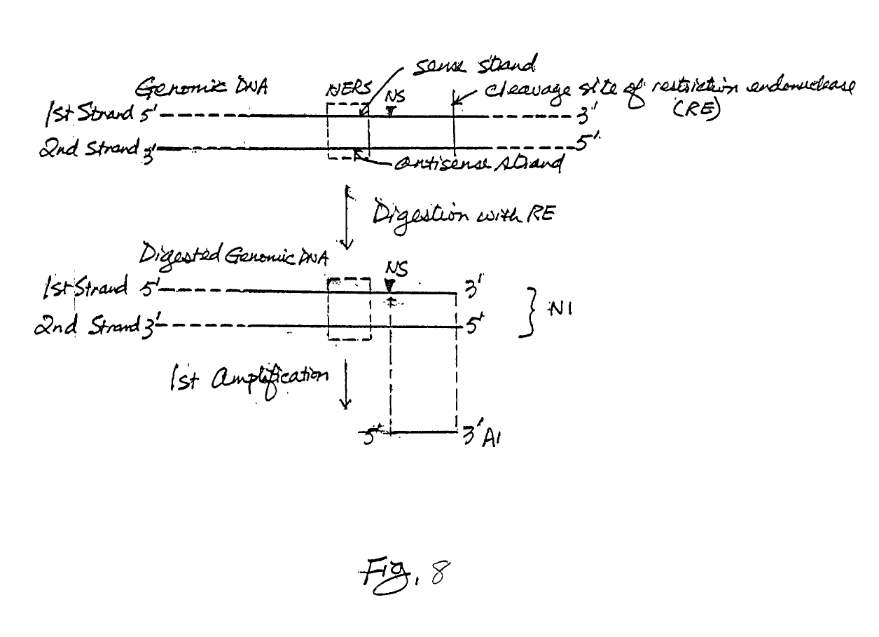

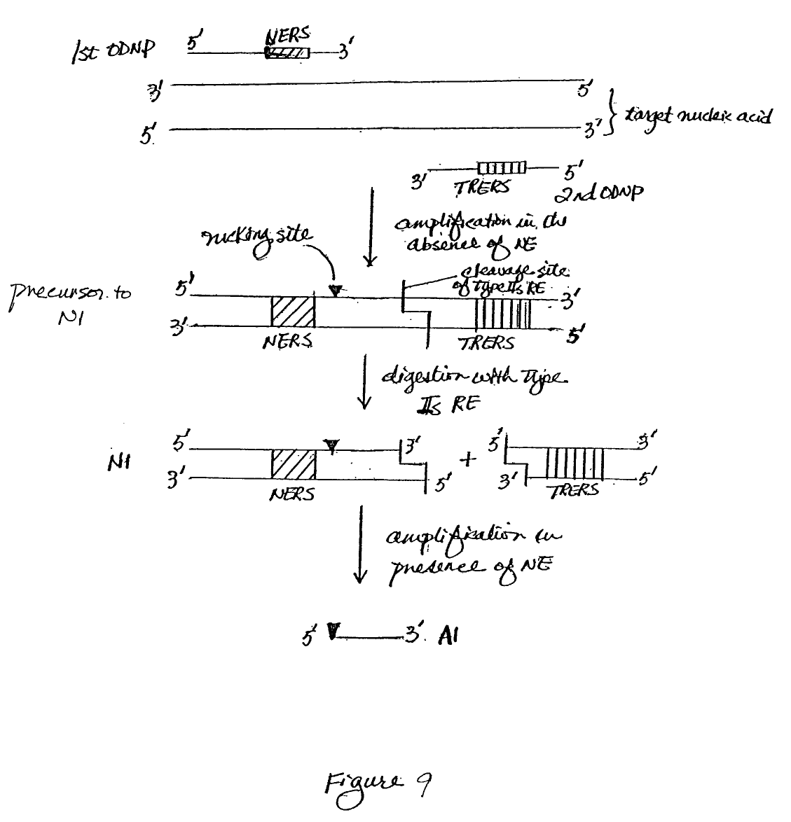

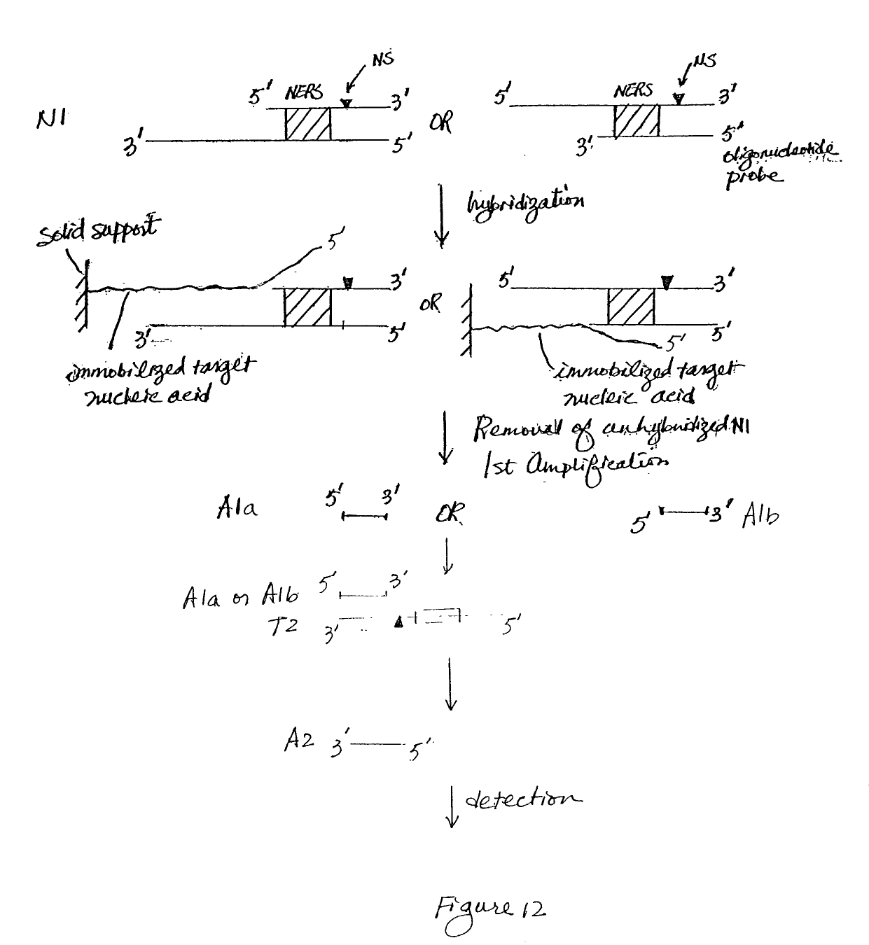

Initial nucleic acid molecules useful for diagnostic applications may be provided by various approaches. For instance, N1 may be obtained by annealing of a trigger ODNP to a T1 molecule where the trigger ODNP is derived from a nucleic acid molecule originated from a pathogenic organism (e.g., FIGS. 5-7 and FIG. 13). Alternatively, N1 may be directly derived from a double-stranded nucleic acid molecule originated from a pathogenic organism (e.g., FIG. 8). N1 may also be prepared by the use of appropriate oligonucleotide primer pairs (e.g., FIGS. 9-11). In certain embodiments, N1 may be a partially double-stranded nucleic acid molecule having an overhang capable of hybridizing with a target nucleic acid (e.g., FIG. 12). These and other means for providing N1 relevant to diagnostic applications are described below.

a. First Type of Exemplary Methods for Providing N1 Molecules

In certain embodiments of the present invention where N1 is provided by annealing a trigger ODNP to a T1 molecule, the trigger ODNP may be derived from either a DNA molecule (e.g., a genomic DNA molecule) or a RNA molecule (e.g., a mRNA molecule) of a pathogenic organism. If the nucleic acid molecule from a pathogenic organism is single-stranded, it may be directly used as a trigger ODNP. Alternatively, the single-stranded nucleic acid may be cleaved to produce shorter fragments, where one or more of these fragments may be used as a trigger ODNP. If the nucleic acid molecule from a pathogenic organism is double-stranded, it may be denatured and directly used as a trigger ODNP or the denatured product may be cleaved to provide multiple shorter single-stranded fragments where one or more of these fragments may function as an ODNP trigger. Alternatively, it may be first cleaved to obtain multiple shorter double-stranded fragments, and the shorter fragments are then denatured to provide one or more trigger ODNPs.

As discussed above, a T1 molecule must be at least substantially complementary to the trigger ODNP. In addition, the number of T1 molecules in an amplification reaction mixture is preferably greater than that of the trigger ODNP to effectively compete with the complementary strand of the trigger ODNP originated from the double-stranded nucleic acid molecule for annealing to the trigger ODNP.

An example of the first type of methods for preparing N1 molecules is shown in FIG. 5. As indicated in this figure, a double-stranded genomic DNA may be first cleaved by a restriction endonuclease. The digestion products may be denatured and one strand of one of the digestion products may be used as a trigger ODNP to initiate nucleic acid amplification reactions.

b. Second Type of Exemplary Methods for Providing N1 Molecules

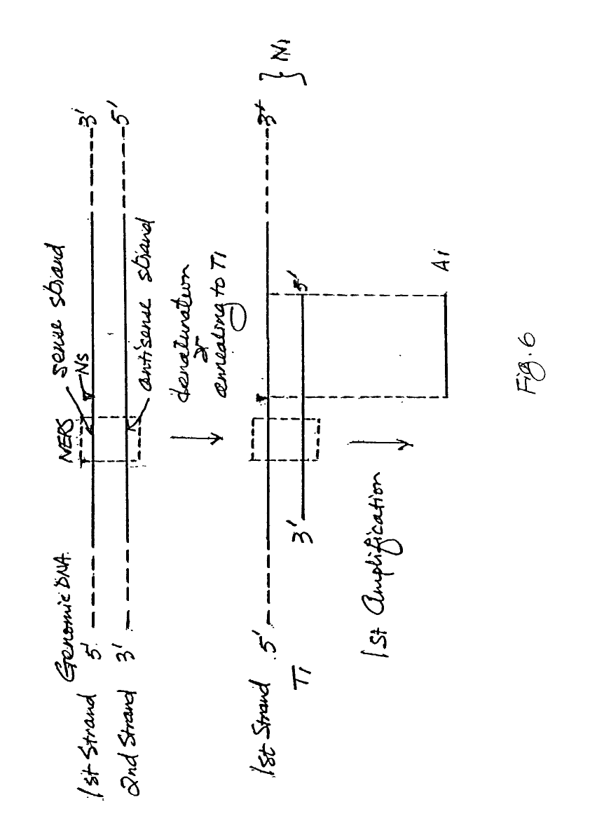

In certain embodiments of the present invention where N1 is provided by annealing a trigger ODNP to a T1 molecule, the trigger ODNP comprises the sequence of the sense strand of a NARS. The trigger ODNP may be derived from a target nucleic acid (e.g., a genomic nucleic acid) originated from a pathogenic organism. A specific embodiment where N1 comprises a NERS recognizable by a nicking endonuclease that nicks outside its recognition sequence (e.g., N.BstNB I) is illustrated in FIG. 6. As illustrated by this figure, a genomic DNA or a fragment thereof comprising a NERS is denatured and one strand of the genomic DNA or a fragment of that strand anneals to a T1 molecule. The T1 molecule is a portion of the other strand of the genomic DNA that comprises a sequence of the antisense strand of the NERS. The annealing of the trigger ODNP to the T1 molecule provides the initial nucleic acid molecule N1 for amplification reactions. The number of T1 molecules in an amplification reaction mixture is preferably greater than the number of strands of genomic DNA or fragments thereof that contain the sequence of the sense strand of the NERS.

The above genomic DNA may be immobilized to a solid support in certain embodiments. In other embodiments, the T1 molecule may be immobilized to a solid support.

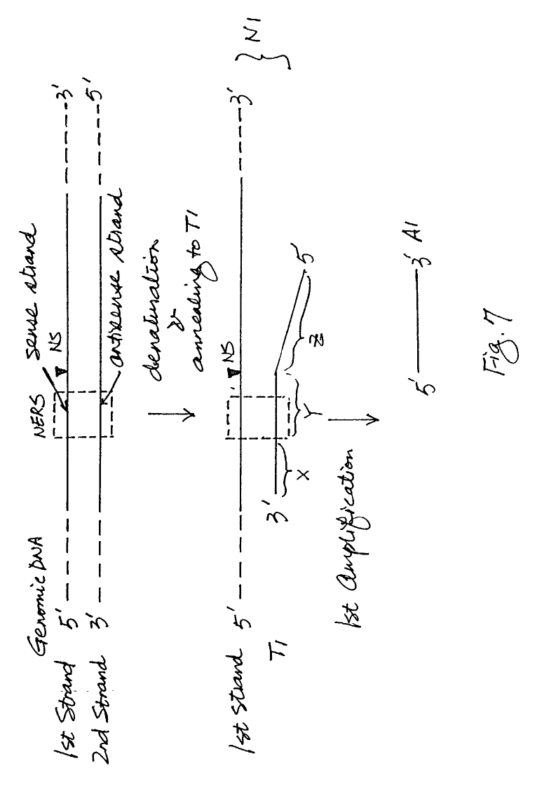

In related embodiments where the trigger ODNP is derived from a target nucleic acid and comprises the sequence of the sense strand of a NARS, a T1 molecule may be at least substantially complementary to the trigger ODNP at its 3′ portion (i.e., Regions X and Y), but not at its 5′ portion (i.e., Region Z) (FIG. 7). The 3′ portion of T1 includes the sequence of the antisense strand of the NARS so that the initial nucleic acid formed by annealing T1 to the trigger ODNP comprises a double-stranded NARS. In the presence of a NA that recognizes the NARS, the N1 molecule is nicked. The 3′ terminus at the nicking site is then extended using a region 5′ to the sequence of the antisense strand of the NARS in the T1 molecule as the template. The resulting amplification product is a single-stranded nucleic acid molecule that is complementary to a region of T1 located 5′ to the sequence of the antisense strand of the NARS (i.e., Region Z1) rather than a portion of the trigger ODNP.

c. Third Type of Exemplary Methods for Providing N1 Molecules

In certain embodiments of the present invention, N1 is a double-stranded nucleic acid derived directly from a genomic nucleic acid that contains both a NARS and a RERS. An embodiment with a NERS recognizable by a nicking endonuclease that nicks outside its recognition sequence (e.g., N.BstNB I) as an exemplary NARS is illustrated in FIG. 8. As shown in this figure, a genomic DNA that comprises a NERS and a RERS may be digested by a restriction endonuclease that recognizes the RERS. The digestion product that contains the NERS may function as an initial nucleic acid molecule (N1).

d. Fourth Type of Exemplary Methods for Providing N1 Molecules

In certain embodiments of the present invention, an initial nucleic acid molecule N1 is a completely or partially double-stranded nucleic acid molecule produced using various ODNP pairs. The methods for using ODNP pairs to prepare N1 molecules are described below in connection with FIGS. 9-11.

In one embodiment, a precursor to N1 contains a double-stranded NARS and a RERS. The NARS and RERS are incorporated into the precursor using an ODNP pair. An embodiment with a NERS recognizable by a NE that nicks outside its recognition sequence (e.g., N.BstNB I) as an exemplary NARS, and a type IIs restriction endonuclease recognition sequence (TRERS) as an exemplary RERS is illustrated in FIG. 9. As shown in this figure, a first ODNP comprises the sequence of one strand of a NERS while a second ODNP comprises the sequence of one strand of a TRERS. When these two ODNPs are used as primers to amplify a portion of a target nucleic acid, the resulting amplification product (i.e., a precursor to N1), contains both a double-stranded NERS and a double-stranded TRERS. In the presence of a type IIs restriction endonuclease that recognizes the TRERS, the amplification product is digested to produce a nucleic acid molecule N1 that comprises a double-stranded NERS.

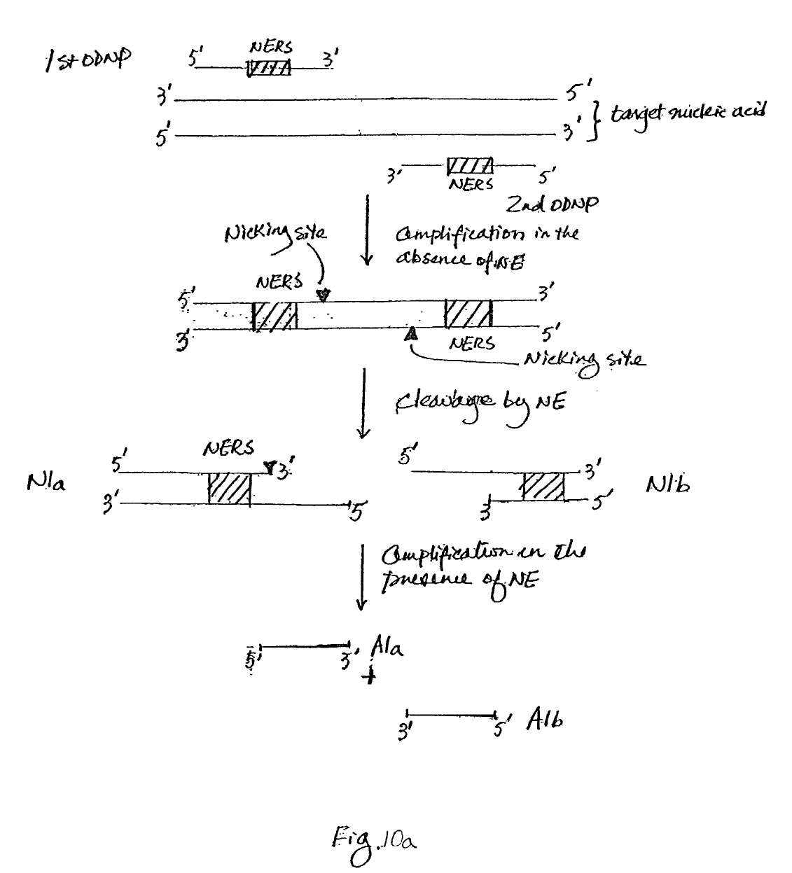

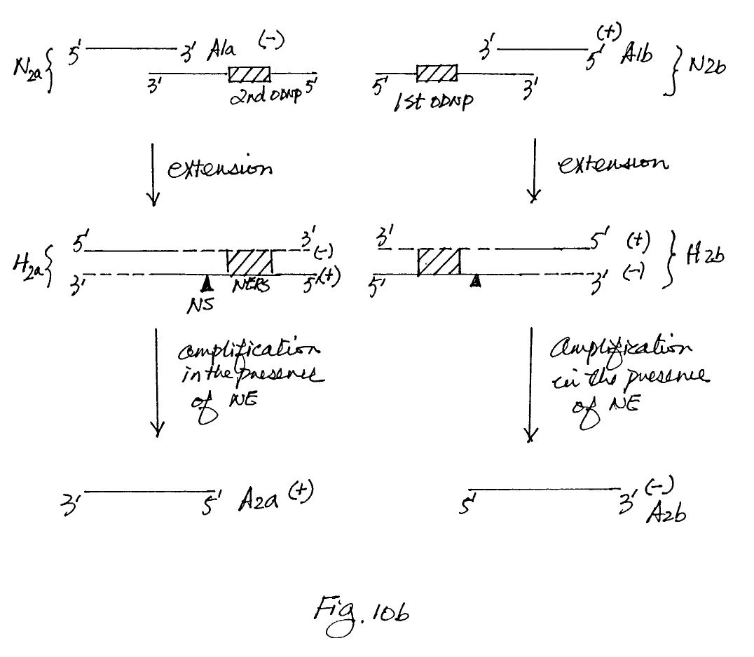

In another embodiment, a precursor to N1 contains two double-stranded NARSs. The two NARSs are incorporated into the precursor to N1 using two ODNPs. An embodiment with a NERS recognizable by a nicking endonuclease that nicks outside its recognition sequence as an exemplary NARS is illustrated in FIG. 10a. As shown in this figure, both ODNPs comprise a sequence of a sense strand of a NERS. When these two ODNPs are used as primers to amplify a portion of a target nucleic acid, the resulting amplification product contains two NERSs. These two NERSs may or may not be identical to each other, but preferably, they are identical. In the presence of a NE or NEs that recognize the NERSs, the amplification product is nicked twice (once on each strand) to produce two nucleic acid molecules (N1a and N1b) that each comprises a double-stranded NERS.

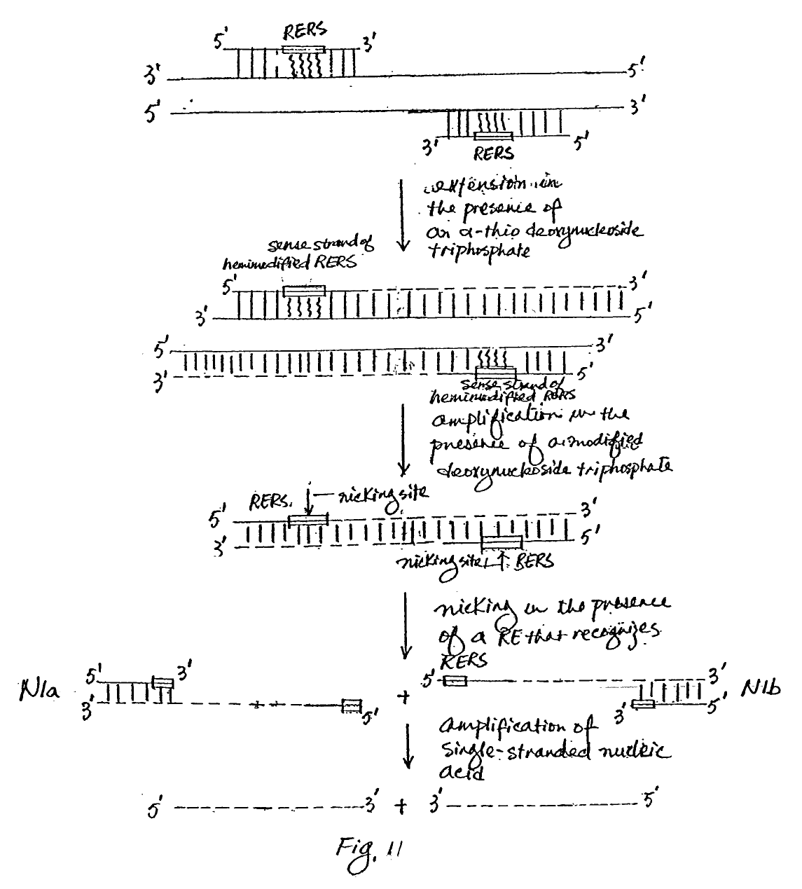

In yet another embodiment, a precursor to N1 contains two hemimodified RERS. The two hemimodified RERSs are incorporated into the precursor by the use of two ODNPs. This embodiment is illustrated in FIG. 11. As shown in this figure, both the first and the second ODNPs comprise a sequence of one strand of a RERS. When these two ODNPs are used as primers to amplify a portion of a target nucleic acid in the presence of a modified deoxynucleoside triphosphate, the resulting amplification product contains two hemimodified RERSs. These two hemimodified RERS may or may not be identical to each other. In the presence of a RE or REs that recognize the hemimodified RERS, the above amplification product is nicked to produce two partially double-stranded nucleic acid molecule (N1a and N1b) that each comprises a sequence of at least one strand of the hemimodified RERS.

The above first ODNP, the second ODNP or both may be immobilized to a solid support in certain embodiments. In other embodiments, the nucleic acid molecules of a sample, including the target nucleic acid are immobilized.

e. Fifth Type of Exemplary Methods for Providing N1 Molecules

In other embodiments of the present invention, an initial nucleic acid molecule N1 is a partially double-stranded nucleic acid molecule having a NARS and an overhang at least substantially complementary to a target nucleic acid. An exemplary embodiment wherein N1 has a NERS recognizable by a nicking endonuclease that nicks outside its recognition sequence as an exemplary NARS is illustrated in FIG. 12. As shown in this figure, the N1 molecule may contain a 5′ overhang in the strand that either comprises a NS or forms a NS upon extension. Alternatively, the N1 molecule may contain a 3′ overhang in the strand that neither comprises a NS nor forms a NS upon extension. The overhang of the N1 molecule must be at least substantially complementary to a target nucleic acid molecule so that it can anneal to the target nucleic acid molecule. The annealing of N1 to the target nucleic acid enables the isolation of a complex formed between the target nucleic acid and the N1 molecule (“target-N1 complex”) in those instances where the target nucleic acid is present in a biological sample of interest.

For instance, the nucleic acid molecules in the biological sample may be immobilized to a solid support as shown in FIG. 12. Such immobilization may be performed by any method known in the art, including without limitation, the use of a fixative or tissue printing. A N1 molecule having an overhang that is substantially complementary to a particular target nucleic acid molecule is then applied to the sample. If the target nucleic acid is present in the sample, N1 hybridizes to the target nucleic acid via its overhang. The sample is subsequently washed to remove any unhybridized N1 molecule. In the presence of a DNA polymerase and nicking endonuclease that recognizes the NERS in N1, a single-stranded nucleic acid molecule A1 is amplified. In the further presence of a suitable T2 molecule, another single-stranded nucleic acid molecule A2 is amplified. However, if the target nucleic acid is absent in the sample, N1 is unable to hybridize to any nucleic acid molecule in the sample and thus is washed off from the sample. Thus, when the washed biological sample is incubated with a nucleic acid amplification reaction mixture (i.e., a mixture containing all the necessary components for single strand nucleic acid amplification using a portion of N1 as a template, such as a NE that recognizes the NERS in the N1 molecule and a DNA polymerase), no single-stranded nucleic acid molecule that is complementary to the above portion of N1 is amplified.

Besides immobilizing a target nucleic acid molecule, a target-N1 complex may be purified by first hybridizing the N1 molecule with the target nucleic acid molecule in a biological sample and then isolating the complex by a functional group associated with the target nucleic acid. For instance, the target nucleic acid may be labeled with a biotin molecule, and the target-N1 complex may be subsequently purified via the biotin molecule associated with the target, such as precipitating the complex with immobilized streptavidin.

In certain related embodiments, N1 is formed by hybridizing an immobilized target nucleic acid from a biological sample with a single-stranded T1 molecule. An example of these embodiments is where a target nucleic acid is not immobilized, but a T1 molecule as described above is immobilized to a solid support via its 5′ terminus. If a target nucleic acid is present in a sample, the hybridization of the nucleic acids of the sample to the T1 allows the target to remain attached to the solid support when the solid support is washed. In the presence of a nicking agent that recognizes the nicking agent recognition sequence of which the antisense strand is present in the T1 and a DNA polymerase, a single-stranded nucleic acid molecule is amplified using a sequence located 5′ to the sequence of the antisense strand of the recognition sequence in the T1 as a template. If the target is absent in the sample, the nucleic acids of the sample will be washed off the solid support to which the T1 is attached. Thus, no single-stranded nucleic acid molecule is amplified using a portion of the T1 as a template.

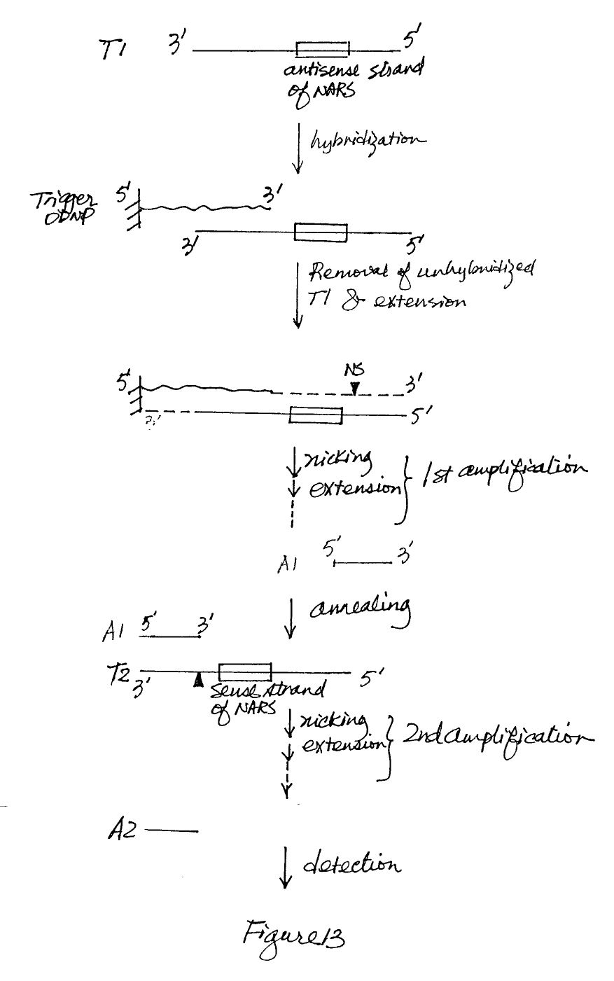

Another example of the above embodiments using a NARS recognizable by a nicking agent that nicks outside the NARS is illustrated in FIG. 13. As shown in this figure, nucleic acids of a biological sample are immobilized via their 5′ termini. The resulting immobilized nucleic acids are then hybridized with a T1 molecule that comprises, from 3′ to 5′, a sequence that is at least substantially complementary to a target nucleic acid suspected to be present in the biological sample and a sequence of the antisense strand of a NARS. If the target nucleic acid is present in the biological sample, the T1 molecule hybridizes to the target nucleic acid to form a N1 molecule. The N1 molecule is separated from unhybridized T1 molecule by washing the solid phase to which the target nucleic acid is attached. In the presence of a DNA polymerase and a nicking agent that recognizes the NARS, N1 is used as a template to amplify a single-stranded nucleic acid molecule A1. However, if the target nucleic acid is absent in the sample, T1 is unable to hybridize to any nucleic acid molecule in the sample and thus is washed off from the solid support. Consequently, no N1 can be formed that attaches to the solid support, and no single-stranded nucleic acid molecule complementary to a portion of N1 can be amplified.

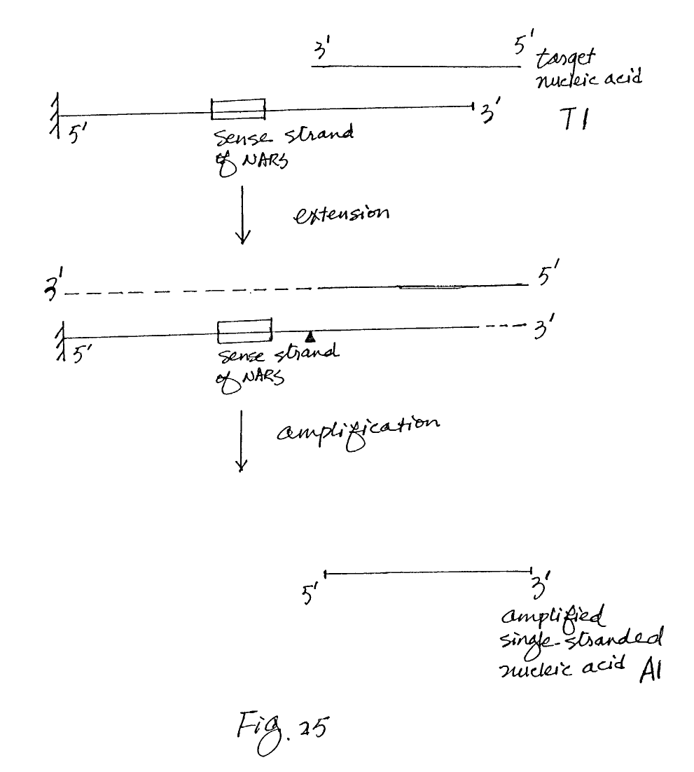

Another example of the above embodiments using a NARS recognizable by a nicking agent that nicks outside the NARS is illustrated in FIG. 25. As shown in this figure, a T1 molecule is immobilized to a solid support via its 5′ terminus. The T1 molecule comprises, from 5′ to 3′, a sequence of the sense strand of the NARS and a sequence that is substantially complementary to the 3′ portion of the target nucleic acid. The T1 molecule is mixed with the nucleic acids from a biological sample. If the target nucleic acid is present in the sample, the T1 molecule is hybridized to the target to form a template molecule. When the solid support to which the T1 molecule is attached is washed, the target remains attached to the solid support via its hybridization with the T1 molecule. In the presence of a DNA polymerase, the target extends from its 3′ terminus using the T1 molecule as a template. The duplex formed between the extension product of the target and that of the T1 molecule comprises a double-stranded NARS. In the presence of a nicking agent that recognizes the NARS as well as the DNA polymerase, a single-stranded nucleic acid molecule is amplified using a portion of the target nucleic acid as a template. However, if the target nucleic acid is absent in the sample, the T1 molecule will not be able to hybridize with the target. Thus, no single-stranded nucleic acid molecule will be amplified using the target as a template.

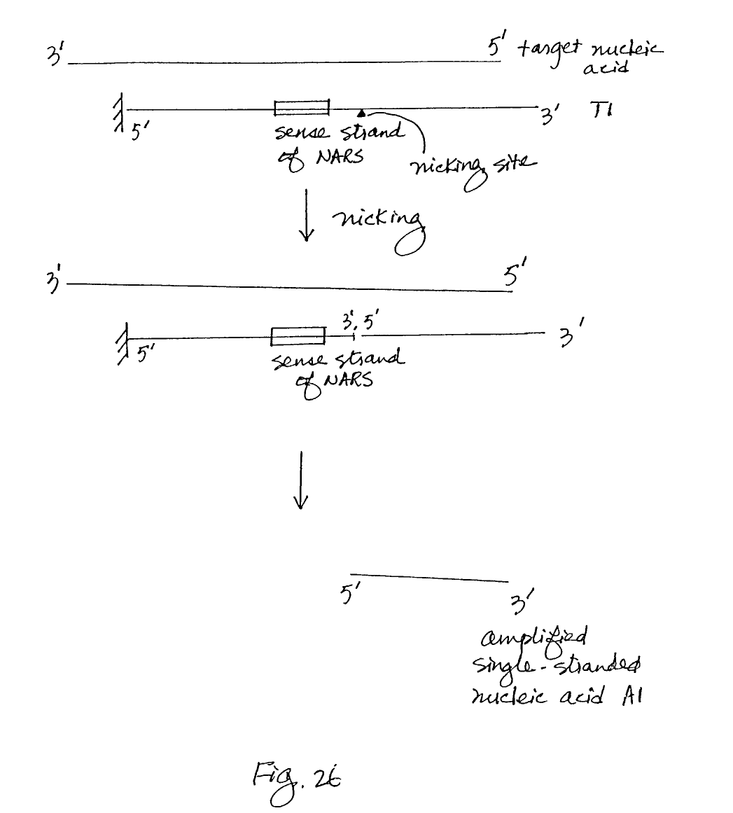

Another example of the above embodiments is illustrated in FIG. 26. In this example, the immobilized T1 molecule is substantially complementary to the target nucleic acid, but not necessarily complementary to the 3′ portion of the target. The T1 also comprises a sequence of the sense strand of a nicking agent recognition sequence. If the target is present in a biological sample, when the T1 molecule is mixed with the nucleic acids in the sample, it may hybridize with the target. When the solid support to which the T1 is attached is washed, the target remains attached to the solid support via its hybridization with the T1. In the presence of a DNA polymerase, and a nicking agent that recognizes the NARS, even when one or more nucleotides in the sequence of the sense strand of the NARS may not form conventional base pairs with nucleotides in the target, in certain circumstances, a single-stranded nucleic acid may be amplified using a portion of the target as a template. The detailed descriptions for the circumstances where a single-stranded nucleic acid is amplified when a template nucleic acid does not comprise a double-stranded NARS are provided in the U.S. Application entitled “Amplification of Nucleic Acid Fragments Using Nicking Agents”. However, if the target nucleic acid is absent in the sample, the probe will not be able to hybridize with the target. Thus, no single-stranded nucleic acid molecule will be amplified using the target as a template.

3. Specificity

The methods of the present invention may be used for detecting the presence or absence of a particular pathogenic organism in a sample, as well as for detecting the presence of several closely related pathogenic organisms. For instance, as to the first and the second types of exemplary methods described above, the portion of a trigger ODNP to which a T1 molecule anneals may be derived from a target nucleic acid or a portion thereof that is specific to a particular pathogenic organism to be detected. Alternatively, such a portion of a trigger ODNP may be derived from a target nucleic acid or a portion thereof that is substantially or completely conserved among several closely related pathogenic organisms, but absent in other more distantly related or unrelated pathogenic organisms.

A target nucleic acid or its portion that is “specific” to a particular pathogenic organism refers to a target nucleic acid or its portion having a sequence present in the particular organism, but not in any other organisms (including those closely related to the particular organism). In addition, a region in a target nucleic acid that is “substantially conserved” among several closely related pathogenic organisms refers to a region in the target nucleic acid for which there exists a nucleic acid molecule capable of hybridizing to the corresponding region in each of the several closely related organisms under appropriate conditions, but incapable of hybridizing to a similar region in the target nucleic acid from a more distantly related or unrelated organism under identical conditions. Also, a region in a target nucleic acid that is “completely conserved” among several closely related pathogenic organisms refers to a region that has an identical sequence in the target nucleic acid from each of the several closely related pathogenic organisms.

Similarly, as to the above fourth type of exemplary methods, the portion of a target nucleic acid that is amplified with a primer pair may be a region that is specific for a particular pathogenic organism, or a region that is substantially or completely conserved among several closely related pathogenic organisms but absent in other distantly related or unrelated pathogenic organisms. In addition, the amplified portion of a target nucleic acid may be a variable region in the target nucleic acid among several closely related pathogenic organisms. As used herein, a “variable” region in a target nucleic acid refers to a region that has less than 50% sequence identity among the target nucleic acids from closely related organisms, but is surrounded by regions at each side having higher than 80% sequence identity among the target nucleic acids from the same closely related organisms. As used herein, percent sequence identity of two nucleic acids is determined using BLAST programs of Altschul et al. (J. Mol. Biol-215: 403-10, 1990) with their default parameters. These programs implement the algorithm of Karlin and Altschul (Proc. Natl. Acad. Sci. USA 87:2264-8, 1990) modified as in Karlin and Altschul (Proc. Natl. Acad. Sci. USA 90:5873-7, 1993). BLAST programs are available, for example, at the web site http://www.ncbi.nlm.nih.gov.

Likewise, as to the above fifth type of exemplary methods, the overhang of a N1 molecule may be at least substantially complementary to a region in a target nucleic acid specific to a pathogenic organism, or a region in a target nucleic acid that is substantially or completely conserved among several closely related pathogenic organisms. When the overhang is completely complementary to a target nucleic acid or a portion thereof from a particular organism, but also substantially complementary to the target nucleic acid or a portion thereof from one or more closely related organisms, one can vary hybridization stringencies to either detect the presence of the particular organism or to detect the presence of any one of the closely related organisms. For example, when a N1 molecule is hybridized with nucleic acids from a biological sample under highly stringent conditions, nucleic acid amplification following the removal of unhybridized N1 molecules using a portion of the N1 molecule as a template may indicate the presence of the particular organism in the biological sample. On the other hand, when a N1 molecule is hybridized with nucleic acids from a biological sample under moderately or low stringent conditions, nucleic acid amplification (following the removal of unhybridized N1 molecules using a portion of the N1 molecule as a template) may indicate a presence of the particular organism and/or one or more organisms closely related to the particular organism. Adjusting stringencies of hybridization conditions is well known in the art and detailed discussions may be found, for example, Sambrook and Russell, Molecular Cloning: A Laboratory Manual, Cold Spring Harbor Press, 2001.

In the embodiments where an initial nucleic acid molecule (N1) is provided by annealing a trigger ODNP to a T1 molecule, the trigger ODNP or a portion thereof and a portion of the T1 molecule located 3′ to the sequence of one strand of a NARS in T1 may be substantially complementary, rather than completely complementary, to each other. For instance, when a trigger ODNP is derived from a region of a target nucleic acid that is substantially conserved among several closely related pathogenic organisms and the presence of any of the several organisms needs to be detected, a T1 molecule substantially complementary to the trigger ODNP may be used. In such a circumstance, the primer extension reaction needs to be performed under conditions that are not too stringent to prevent the trigger ODNP from annealing to the T1 molecule or prevent the trigger ODNP from being extended using a portion of the T1 molecule as a template. However, such conditions need also be sufficiently stringent to prevent the T1 molecule from non-specifically annealing to a nucleic acid molecule other than the trigger ODNP. Conditions suitable for nucleic acid amplification where a trigger ODNP or a portion thereof is substantially complementary to a portion of a T1 molecule may be worked out by adjusting the reaction temperature and/or reaction buffer composition or concentration. Generally, similar to hybridization reactions, an increase in reaction temperatures increases the stringency of amplification reactions.

4. A1 Molecules

As described above, an A1 molecule is amplified using a portion of N1 as a template. In certain embodiments, A1 may be relatively short and has at most 25, 20, 17, 15, 10, or 8 nucleotides. Such short length may be accomplished by appropriately designing T1 molecules or ODNPs used in making N1 molecules. For instance, for the second type of providing N1 molecules (FIG. 6), T1 may be designed to have a short region 5′ to a sequence of the antisense strand of a NARS. For the fourth type of providing N1 molecules (FIGS. 9-11), the ODNP pair may be designed to be close to each other when the primers anneal to the target nucleic acid. The short length of an A1 molecule may be advantageous because it increases amplification efficiencies and rates. In addition, it allows the use of a DNA polymerase that does not have a stand displacement activity. It also facilitates the detection of A1 molecules and/or a product (A2) of a subsequent amplification reaction in which A1 is used as an initial amplification primer via certain technologies such as mass spectrometric analysis.

5. T2 Molecules

A T2 molecule of the present invention comprises the sequence of the sense strand of a NARS as well as a sequence, located 3′ to the sequence of the sense strand of the NARS, that is at least substantially complementary to a single-stranded nucleic acid molecule (A1) amplified using a portion of an initial nucleic acid molecule N1 as a template. Preferably, a T2 molecule comprises a sequence that is completely complementary to an A1 molecule.

Also as discussed above, in the above fourth type of exemplary methods, the portion of a target nucleic acid that is amplified with a primer pair may be a region that is specific for a particular pathogenic organism, or a region that is substantially or completely conserved among several closely related pathogenic organisms but absent in other distantly related or unrelated pathogenic organisms. In addition, the amplified portion of a target nucleic acid may be a variable region in the target nucleic acid among several closely related pathogenic organisms. When the amplified region is substantially conserved, one may use a T2 molecule comprising a sequence, located 3′ to the sequence of the antisense strand of a NARS, that is identical to one strand of the amplified region from a particular organism to detect the presence of the particular organism by performing the amplification reaction under highly stringent conditions (e.g., a relatively high amplification temperature to prevent an A1 molecule derived from an organism other than the particular organism from hybridizing with the T2 molecule). Alternatively, one may use the same T2 molecule to detect the presence of the particular organism as well as the presence of one or more organisms closely related to the particular organism by performing the amplification reaction under moderately or low stringent conditions (e.g., a relatively low amplification temperature to allow an A1 molecule derived from an organism closely related to the particular organism to hybridize with the T2 molecule and to be extended using a portion of the T2 molecule as a template).

Additionally, in the embodiments where the amplified region is a variable region among closely related organisms, a T2 molecule may comprise a sequence that is at least substantially complementary to an A1 molecule amplified using a N1 molecule derived from a particular organism among the above closely related organisms. The amplification of a single-stranded nucleic acid molecule using a portion of the T2 molecule as a template indicates the presence of the particular organism in a biological sample.

In certain embodiments, no additional T2 molecules are needed for a second amplification reaction. In these embodiments, the second ODNP used in producing a N1 molecule has a 3′ terminal sequence that allows the second ODNP to anneal to A1. The second ODNP also comprises a sequence of the sense strand of a NARS. Thus, the extension of A1 using the second ODNP as a template creates a double-stranded NARS. In the presence of a DNA polymerase and a NA that recognizes the NARS, a single-stranded nucleic acid (A2) is amplified using A1 as a template. An example of the above embodiments is illustrated in FIG. 10b where A1a and A1b are amplified in a first amplification reaction that uses two ODNPs each comprising a sequence of the sense strand of a NERS (FIG. 10a).

A T2 molecule may be immobilized to a solid support, preferably at its 5′ terminus, in certain embodiments. In other embodiments, a T2 molecule may not be immobilized.

6. Detecting and/or Characterizing Amplified Single-Stranded Nucleic Acids

The presence of a target nucleic acid originated from a pathogenic organism may be detected by detecting and/or characterizing an amplification product (e.g., A1, A2, etc.). Any methods suitable for detecting or characterizing single-stranded nucleic acid molecules may be used. For instance, the amplification reaction may be carried out in the presence of a labeled deoxynucleoside triphosphate so that the label is incorporated into the amplified nucleic acid molecules. Labels suitable for incorporating into a nucleic acid fragment, and methods for the subsequent detection of the fragment are known in the art, and exemplary labels include, but are not limited to, a radiolabel such as ³²P, ³³P, ¹²⁵I or ³⁵S, an enzyme capable of producing a colored reaction product such as alkaline phosphatase, fluorescent labels such as fluorescein isothiocyanate (FITC), biotin, avidin, digoxigenin, antigens, haptens, or fluorochromes.

Alternatively, amplified nucleic acid molecules may be detected by the use of a labeled detector oligonucleotide that is substantially, preferably completely, complementary to the amplified nucleic acid molecules. Similar to a labeled deoxynucleoside triphosphate, the detector oligonucleotide may also be labeled with a radioactive, chemiluminescent, or fluorescent tag (including those suitable for detection using fluorescence polarization or fluorescence resonance energy transfer), or the like. See, Spargo et al., Mol. Cell. Probes 7: 395-404, 1993; Hellyer et al., J. Infectious Diseases 173: 934-41, 1996; Walker et al., Nucl. Acids Res. 24: 348-53, 1996; Walker et al., Clin. Chem. 42: 9-13, 1996; Spears et al., Anal. Biochem. 247: 130-7, 1997; Mehrpouyan et al., Mol. Cell. Probes 11: 337-47, 1997; and Nadeau et al., Anal. Biochem. 276: 177-87, 1999.

In certain embodiments, amplified nucleic acid molecules may be further characterized. The characterization may confirm the identities of these nucleic acid molecules and thus confirm the presence of a target nucleic acid from a pathogenic organism in a biological sample. Such a characterization may be performed via any known method suitable for characterizing single-stranded nucleic acid fragments. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445, incorporated herein in their entireties.

Besides detecting and/or characterizing an amplification product to detect the presence of a target nucleic acid in a biological sample, the presence of the target nucleic acid may be detected by detecting completely or partially double-stranded nucleic acid molecules produced in the amplification reactions (e.g., H1, H2 or nicking product thereof). In a preferred embodiment, the detection of the double-stranded nucleic acid molecule may be performed by adding to the amplification mixture a dye that specifically binds to double-stranded nucleic acid molecules and becomes fluorescent upon binding to double-stranded nucleic acid molecules (i.e., fluorescent intercalating agent). The addition of a fluorescent intercalating agent enables real time monitoring of nucleic acid amplification. Alternatively, to maximize the production of double-stranded nucleic acid molecules (e.g., H1 and H2), the NE, but not the DNA polymerase, in the nicking-extension reaction mixture may be inactivated (e.g., by heat treatment). The inactivation of the NE allows all the nicked nucleic acid molecules in the reaction mixture to be extended to produce double-stranded nucleic acid molecules.

Various fluorescent intercalating agents are known in the art and may be used in the present invention. Exemplary agents include, without limitation, those disclosed in U.S. Pat. Nos. 4,119,521; 5,599,932, 5,658,735; 5,734,058; 5,763,162; 5,808,077; 6,015,902; 6,255,048 and 6,280,933, those discussed in Glazer and Rye, Nature 359: 859-61, 1992, PicoGreen dye, and SYBR® dyes such as SYBR® Gold, SYBR® Green I and SYBR® Green II (Molecular Probes, Eugene Wash.). Fluorescence produced by fluorescent intercalating agents may be detected by various detectors, including PMTs, CCD cameras, fluorescent-based microscopes, fluorescent-based scanners, and fluorescent-based microplate readers, fluorescent-based capillary readers.

7. Compositions and Kits Useful in Diagnosis

Compositions and kits useful in pathogen diagnosis may be the same as those described above for exponential amplification of nucleic acids. In certain embodiments, these compositions and kits may further comprise an additional component to facilitate the detection of amplification products. For instance, the additional component may be a labeled deoxynucleoside triphosphate to be incorporated into amplification products. Alternatively, it may be a labeled detector oligonucleotide capable of hybridizing with amplification products. In certain preferred embodiments, the additional component may be a fluorescent intercalating agent.

8. Diagnostic Uses of the Present Invention

The present invention is useful in quickly detecting the presence of any target nucleic acid of interest. In certain embodiments, the target nucleic acid is derived or originated from a pathogenic organism (e.g., an organism that causes infectious diseases). Such pathogenic organisms include those that impose bio-threat, such as Anthrax and smallpox. In addition, as described above, the present methods may be used for the detecting the presence of a particular pathogenic organism as well as for detecting the presence of several closely related pathogenic organisms. The present invention may also be used to detect organisms that are resistant to certain antibiotics. For example, the present methods, compositions or kits may be used to detect certain pathogenic organisms in a subject that has been treated with an antibiotic or certain combinations of antibiotics. Furthermore, the use of fluorescent intercalating agents for detecting nucleic acid amplification in some embodiments offers real time detection of a target nucleic acid in a biological sample.

### D. Use of Nucleic Acid Amplification Methods and Compositions in Genetic Variation Detection

The methods and compositions for exponential nucleic acid amplification may also be used for detecting genetic variations at defined locations in target nucleic acids. A target nucleic acid or its portion that comprises a genetic variation is first incorporated into an initial nucleic acid molecule (N1) to be used as a template in a first amplification reaction. The initial nucleic acid molecule also comprises at least one strand of a first nicking agent recognition sequence and thus allows for the first amplification reaction in the presence of a DNA polymerase and a nicking agent that recognizes the first nicking agent recognition sequence. The product (A1) from the first amplification reaction comprises the nucleotide(s) at the defined location in the target nucleic acid or the complementary nucleotide(s) of the above nucleotide(s). A1 then anneals to another template nucleic acid (T2). T2 comprises a sequence of the sense strand of a second NARS and thus allows for a second amplification reaction in the presence of the DNA polymerase and a nicking agent that recognizes the second NARS. The characterization of A1 and/or A2 enables the identification of the genetic variation in the target nucleic acid.

1. Target Nucleic Acids

The target nucleic acid of the present invention related to identifying genetic variations is any nucleic acid molecule that may contain a genetic variation using a wild type nucleic acid sequence as a reference. It may or may not be immobilized to a solid support. It can be either single-stranded or double-stranded. A single-stranded target nucleic acid may be one strand of a denatured double-stranded DNA. Alternatively, it may be a single-stranded nucleic acid not derived from any double-stranded DNA. In one aspect, the target nucleic acid is DNA, including genomic DNA, ribosomal DNA and cDNA. In another aspect, the target is RNA, including mRNA, rRNA and tRNA.

In one aspect, the target nucleic acid either is or is derived from naturally occurring nucleic acid. A naturally occurring target nucleic acid is obtained from a biological sample. Preferred biological samples include one or more mammalian tissues, preferably human tissues, (for example blood, plasma/serum, hair, skin, lymph node, spleen, liver, etc.) and/or cells or cell lines. The biological samples may comprise one or more human tissues and/or cells. Mammalian and/or human tissues and/or cells may further comprise one or more tumor tissues and/or cells.

Methodology for isolating populations of nucleic acids from biological samples is well known and readily available to those skilled in the art of the present invention. Exemplary techniques are described, for example, in the following laboratory research manuals: Sambrook et al., “Molecular Cloning” (Cold Spring Harbor Press, 3rd Edition, 2001) and Ausubel et al., “Short Protocols in Molecular Biology” (1999) (incorporated herein by reference in their entireties). Nucleic acid isolation kits are also commercially available from numerous companies, and may be used to simplify and accelerate the isolation process.

The target nucleic acid contains one or more nucleotides of unknown identity (i.e., genetic variations). The present invention provides compositions and methods whereby the identity of the unknown nucleotide(s) becomes known and thereby the genetic variation becomes identified. The base(s) of unknown identity is present at the “nucleotide locus” (or the “defined position” or the “defined location”), which refers to a specific nucleotide or region encompassing one, two, three, four, five, six, seven, or more nucleotides having a precise location on a target nucleic acid.

The term “polymorphism” refer to the occurrence of two or more genetically determined alternative sequences or alleles in a small region (i.e., one to several (e.g., 2, 3, 4, 5, 6, 7, or 8) nucleotides in length) in a population. The two or more genetically determined alternative sequences or alleles each may be referred to as a “genetic variation.” The genetic variation may be the allelic form occurring most frequently in a selected population also referred to as “the wild type form” or one of the other allelic forms. Diploid organisms may be homozygous or heterozygous for allelic forms.

Genetic variations may or may not have effects on gene expression, including expression levels and expression products (i.e., encoded peptides). Genetic variations that affect gene expression are also referred to as “mutations,” including point mutations, frameshift mutations, regulatory mutations, nonsense mutations, and missense mutation. A “point mutation” refers to a mutation in which a wild-type base (i.e., A, C, G, or T) is replaced with one of the other standard bases at a defined nucleotide locus within a nucleic acid sample. It can be caused by a base substitution or a base deletion. A “frameshift mutation” is caused by small deletions or insertions that, in turn, cause the reading frame(s) of a gene to be shifted and, thus, a novel peptide to be formed. A “regulatory mutation” refers to a mutation in a non-coding region, e.g., an intron, a region located 5′ or 3′ to the coding region, that affects correct gene expression (e.g., amount of product, localization of protein, timing of expression). A “nonsense mutation” is a single nucleotide change resulting in a triplet codon (where mutation occurs) being read as a “STOP” codon causing premature termination of peptide elongation, i.e., a truncated peptide. A “missense mutation” is a mutation that results in one amino acid being exchanged for a different amino acid. Such a mutation may cause a change in the folding (3-dimensional structure) of the peptide and/or its proper association with other peptides in a multimeric protein.

In one aspect of the invention, the genetic variation is a “single-stranded nucleotide polymorphism” (SNP), which refers to any single nucleotide sequence variation, preferably one that is common in a population of organisms and is inherited in a Mendelian fashion. Typically, the SNP is either of two possible bases and there is no possibility of finding a third or fourth nucleotide identity at an SNP site.

The genetic variation may be associated with or cause diseases or disorders. The term “associated with,” as used herein, refers to the presence of a positive correlation between the occurrence of the genetic variation and the presence of a disease or a disorder in the host. Such diseases or disorders may be human genetic diseases or disorders and include, but are not limited to, cystic fibrosis, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer' s disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

Target nucleic acids may be amplified before being incorporated into initial nucleic acids as described below. Any of the known methods for amplifying nucleic acids may be used. Exemplary methods include, but are not limited to, the use of Qbeta Replicase, Strand Displacement Amplification (Walker et al., Nucleic Acid Research 20: 1691-6, 1995), transcription-mediated amplification (Kwoh et al., PCT Int'l. Pat. Appl. Pub. No. WO88/10315), RACE (Frohman, Methods Enzymol. 218:340-56, 1993), one-sided PCR (Ohara et al., Proc. Natl. Acad. Sc. 86: 5673-7, 1989), and gap-LCR (Abravaya et al., Nucleic Acids Res. 23: 675-82, 1995). The cited articles and the PCT international patent application are incorporated herein by reference in their entireties.

2. Initial Nucleic Acid Molecules (N1)

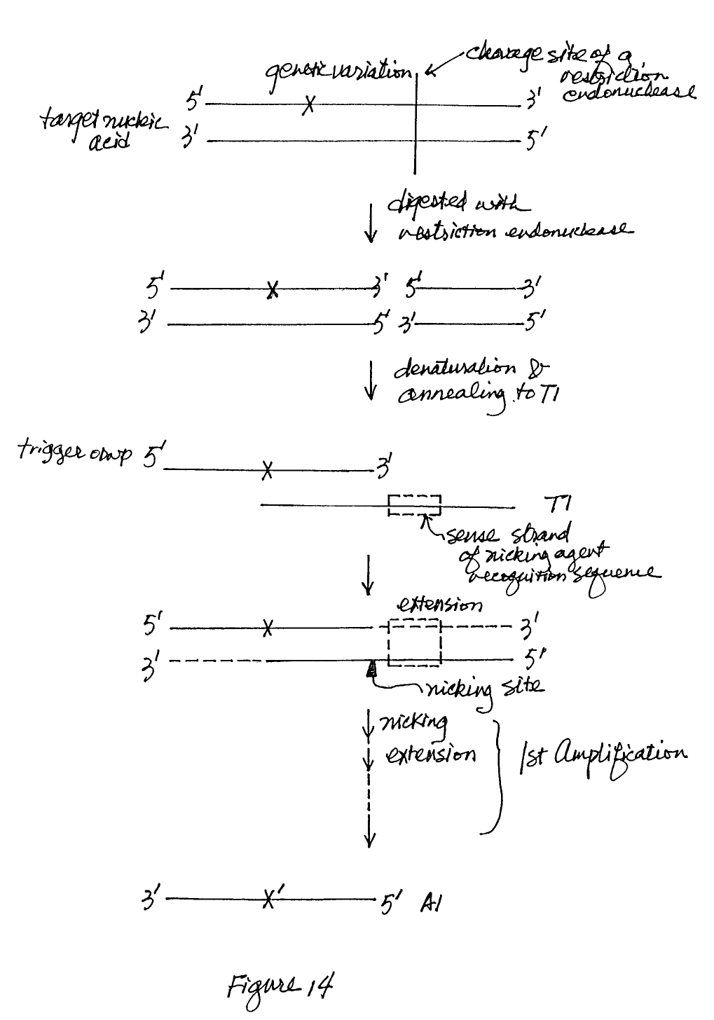

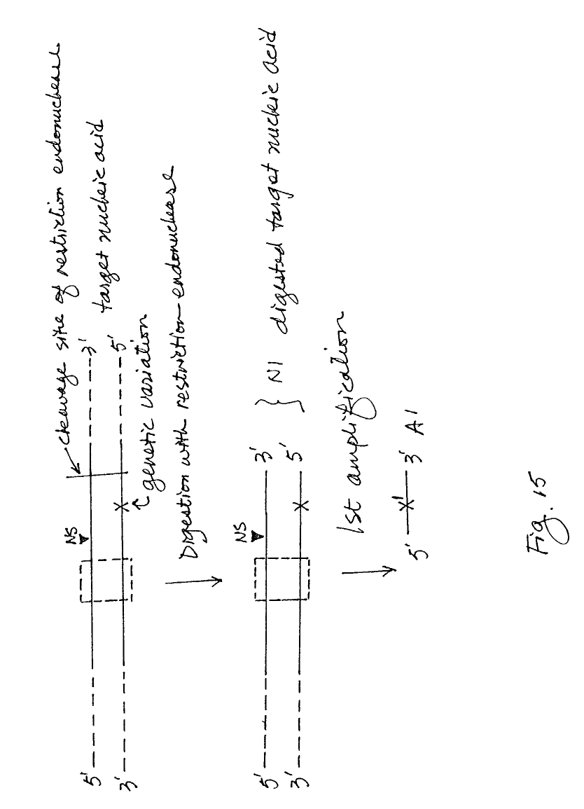

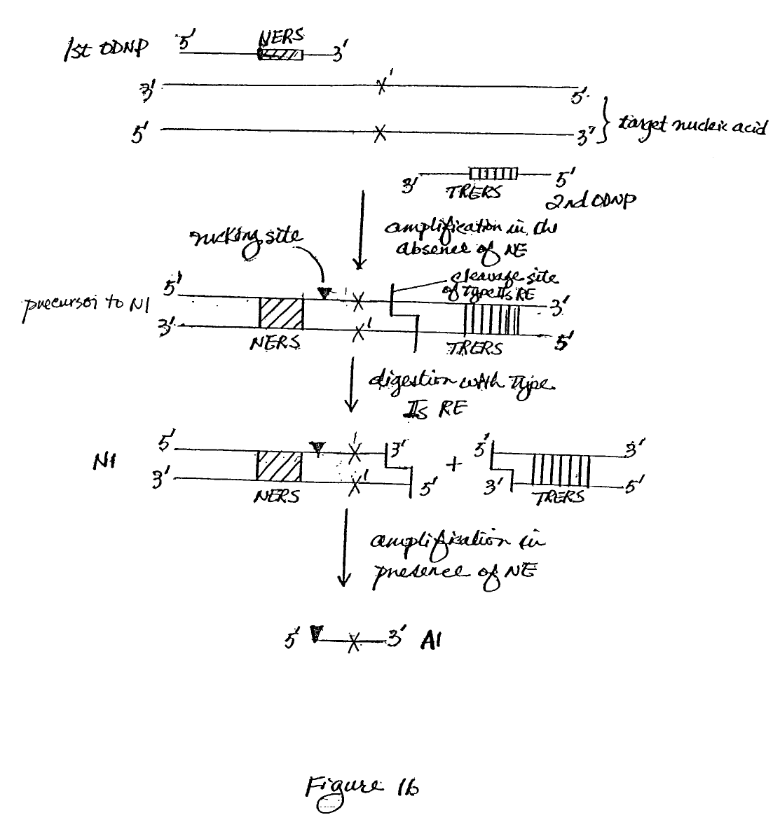

Initial nucleic acid molecules useful for genetic variation detection may be provided by various approaches. For instance, N1 may be obtained by annealing of a trigger oligonucleotide primer to a T1 molecule where the trigger primer is derived from a target nucleic acid and encompasses a genetic variation in the target nucleic acid (e.g., FIG. 14). Alternatively, N1 may be directly derived from a double-stranded target nucleic acid (e.g., by digestion of the target nucleic acid with a restriction endonuclease as shown in FIG. 15). N1 may also be prepared by the use of appropriate oligonucleotide primer pairs (e.g., FIGS. 16-18). Several exemplary means for providing initial nucleic acid molecules are described below.

a. First Type of Exemplary Methods for Providing N1 Molecules

As noted above, N1 may be provided by annealing a trigger oligonucleotide primer to a T1 molecule. The trigger primer needs to encompass genetic variation of a target nucleic acid. An example of this type of methods for providing N1 molecules is illustrated in FIG. 14. As shown in this figure, a double-stranded target nucleic acid (e.g., a genomic DNA) is first cleaved by a restriction endonuclease whose recognition sequence is close to the defined location where a genetic variation is present. The digestion products may be denatured and the strand of the digestion product that comprises the potential genetic variation may then be used as a trigger oligonucleotide primer to anneal to a template nucleic acid (T1). T1 comprises a sequence of the sense strand of a nicking agent recognition sequence so that in the presence of a DNA polymerase and a nicking agent that recognizes the recognition sequence, a single-stranded nucleic acid fragment (A1) is amplified that comprises the complementary nucleotide(s) of the genetic variation of the target nucleic acid.

b. Second Type of Exemplary Methods for Providing N1 Molecules

In certain embodiments of the present invention, N1 is directly derived from a target nucleic acid that comprises a potential genetic variation, a nicking agent recognition sequence, and a restriction endonuclease recognition sequence. An embodiment with a recognition sequence recognizable by a nicking endonuclease that nicks outside its recognition sequence (e.g., N.BstNB I) as an exemplary nicking agent recognition sequence is illustrated in FIG. 15. As shown in this figure, a target nucleic acid may be digested by a restriction endonuclease that recognizes a sequence in the target nucleic acid. The digestion product that contains the nicking endonuclease recognition sequence may function as an initial nucleic acid molecule (N1) to amplify a single-stranded nucleic acid fragment (A1). The genetic variation (“X”) needs to be between the position corresponding to the nicking site produced by the nicking agent and the restriction cleavage site of the restriction endonuclease. Such a location allows the amplified fragment (A1) to contain the complement (“X′”) of the genetic variation.

c. Third Type of Exemplary Methods for Providing N1 Molecules

In certain embodiments of the present invention, an initial nucleic acid molecule N1 is a completely or partially double-stranded nucleic acid molecule produced using various ODNP pairs. The methods for using ODNP pairs to prepare N1 molecules are briefly described below in connection with FIGS. 16-18. More detailed description may be found in U.S. Prov. Appl. Nos. 60/305,637 and 60/345,445.

In certain embodiments, a precursor to N1 contains a double-stranded nicking agent recognition sequence and a restriction endonuclease recognition sequence. The nicking agent recognition sequence and the restriction endonuclease recognition sequence are incorporated into the precursor using a primer pair. An embodiment with a recognition sequence recognizable by a nicking agent that nicks outside its recognition sequence (e.g., N.BstNB I) as an exemplary nicking agent recognition sequence, and a type IIs restriction endonuclease recognition sequence (TRERS) as an exemplary restriction endonuclease recognition sequence is illustrated in FIG. 16. As shown in this figure, a first primer comprises the sequence of one strand of a nicking agent recognition sequence, while a second ODNP comprises the sequence of one strand of a type IIs restriction endonuclease recognition sequence. When these two ODNPs are used as primers to amplify a portion of a target nucleic acid, the resulting amplification product (i.e., a precursor to N1), contains both a double-stranded NERS and a double-stranded TRERS. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. Such designs allow the precursor to N1 to encompass the genetic variation and its complement. In the presence of a type IIs restriction endonuclease that recognizes the TRERS, the amplification product is digested to produce a partially double-stranded nucleic acid molecule N1 that comprises a double-stranded NERS.

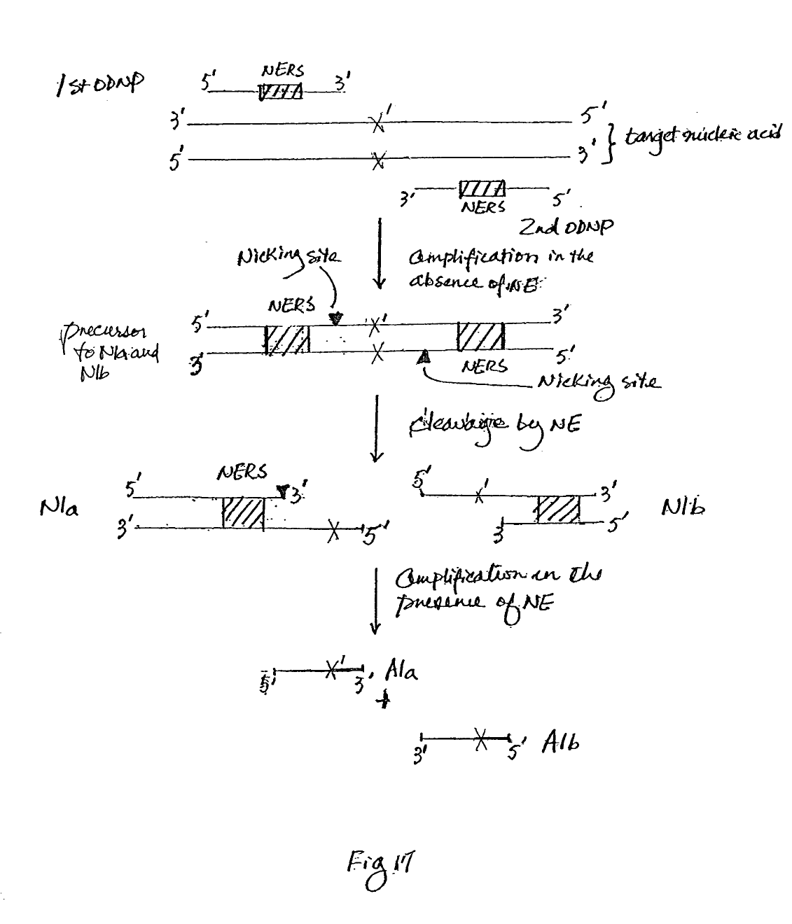

In other embodiments, a precursor to N1 contains two double-stranded nicking agent recognition sequences. The two nicking agent recognition sequences are incorporated into the precursor to N1 using two oligonucleotide primers. An embodiment with a recognition sequence recognizable by a nicking endonuclease that nicks outside its recognition sequence as an exemplary nicking agent recognition sequence is illustrated in FIG. 17. As shown in this figure, both primers comprise a sequence of a sense strand of a nicking endonuclease recognition sequence. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. When these two primers are used as primers to amplify a portion of a target nucleic acid, the resulting amplification product (i.e., a precursor to N1a and N1b described below) contains the genetic variation and its complement, as well as two nicking endonuclease recognition sequences. These two recognition sequences may or may not be identical to each other, but preferably, they are identical. In the presence of a nicking endonuclease or nicking endonucleases that recognize the recognition sequences, the amplification product is nicked twice (once on each strand) to produce two partially double-stranded nucleic acid molecules (N1a and N1b) that each comprises one of the double-stranded nicking endonuclease recognition sequences.

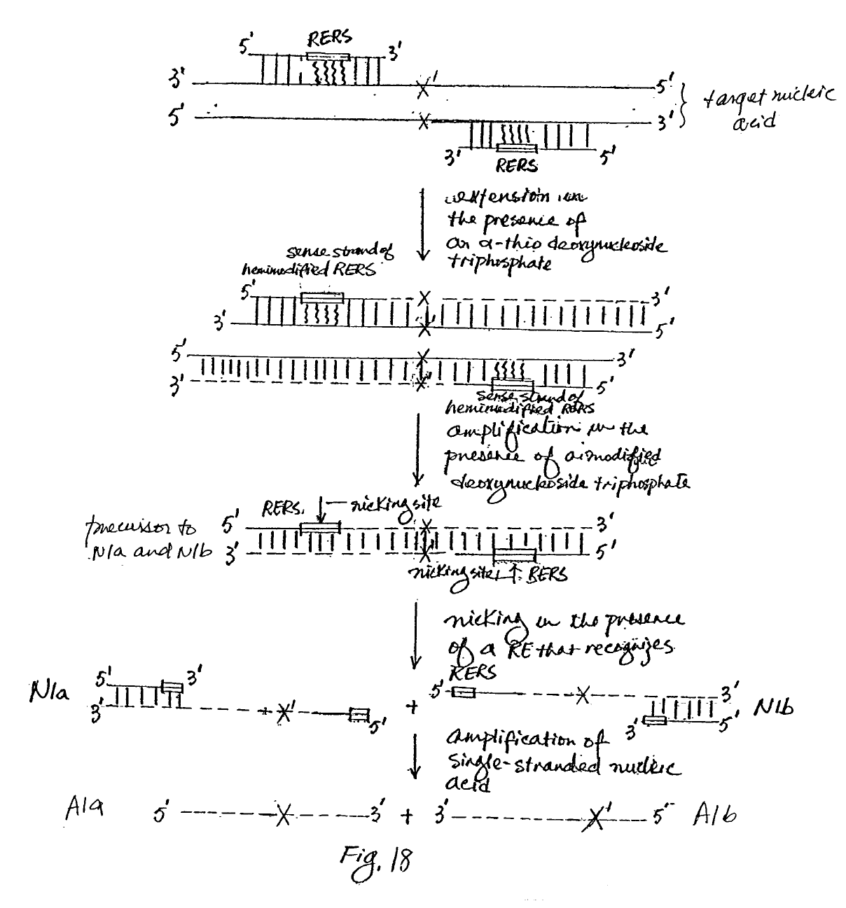

Another embodiment with a hemimodified restriction endonuclease recognition sequence as an exemplary nicking agent recognition sequence is illustrated in FIG. 18. As shown in this figure, both the first and the second primers comprise a sequence of one strand of a restriction endonuclease recognition sequence. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. When these two primers are used as primers to amplify a portion of a target nucleic acid in the presence of a modified deoxynucleoside triphosphate, the resulting amplification product (i.e., a precursor to N1a and N1b described below) contains the genetic variation and its complement, as well as two hemimodified restriction endonuclease recognition sequences. These two hemimodified recognition sequences may or may not be identical to each other. In the presence of a restriction endonuclease or restriction endonucleases that recognize the hemimodified recognition sequences, the above amplification product is nicked to produce two partially double-stranded nucleic acid molecules (N1a and N1b) that each comprises a sequence of at least one strand of one of the hemimodified restriction endonuclease recognition sequences.

The above first ODNP, the second ODNP or both may be immobilized to a solid support in certain embodiments. In other embodiments, the nucleic acid molecules of a sample, including the target nucleic acid are immobilized.

3. A1 Molecules

As described above, an A1 molecule is amplified using a portion of N1 as a template. This portion of N1 comprises the genetic variation or its complement of the target nucleic acid so that A1 comprises the complement of the genetic variation or the genetic variation itself. A1 may be relatively short and has at most 25, 20, 17, 15, 10, or 8 nucleotides. Such short length may be accomplished by appropriately designing oligonucleotide primers used in making N1 molecules. For instance, for the third type of providing N1 molecules (FIGS. 16-18), the ODNP pair may be designed to be close to each other when they anneal to the target nucleic acid. Similar to the diagnostic application of the present invention described above, the short length of an A1 molecule increases amplification efficiencies and rates, allows the use of a DNA polymerase that does not have a stand displacement activity, and facilitates the detection of A1 molecules and/or a product (A2) of a subsequent amplification reaction in which A1 is used as an initial amplification primer via certain technologies such as mass spectrometric analysis.

4. T2 Molecules

A T2 molecule of the present invention comprises a sequence of the sense strand of a NARS as well as a sequence, located 3′ to the sequence of the sense strand of the NARS, that is at least substantially complementary to a single-stranded nucleic acid molecule (A1) amplified using a portion of an initial nucleic acid molecule N1 as a template. Because T2 comprises a sequence of the sense strand of a nicking agent recognition sequence, in the presence of a nicking agent that recognizes the recognition sequence and a DNA polymerase, A1 is used as primer for the initial nucleic acid extension and subsequently used as a template for amplifying another single-stranded nucleic acid fragment (A2). As noted above, A1 comprises a genetic variation or its complement of a target nucleic acid. Thus, A2 comprises the complement of the genetic variation or the genetic variation itself. Accordingly, the characterization of A2 is able to detect and/or identify the genetic variation of the target nucleic acid.

Similar to the diagnostic application of the present invention, in certain embodiments of genetic variation detection according to the present invention, no additional T2 molecules are needed for a second amplification reaction. In these embodiments, the second ODNP used in producing a N1 molecule has a 3′ terminal sequence that allows the second ODNP to anneal to A1. The second ODNP also comprises a sequence of the sense strand of a NARS. Thus, the extension of A1 using the second ODNP as a template creates a double-stranded NARS. In the presence of a DNA polymerase and a NA that recognizes the NARS, a single-stranded nucleic acid (A2) is amplified using A1 as a template.

The T2 molecule may be immobilized to a solid support, preferably via its 5′ terminus, in certain embodiments. In other embodiments, the T2 molecule may not be immobilized.

5. Characterizing Amplified Single-Stranded Nucleic Acids

A potential genetic variation in a target nucleic acid may be detected or identified by characterizing an amplification product (i.e., A1 or A2). Any method suitable for characterizing single-stranded nucleic acid molecules may be used. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445.

Many of the methodologies for characterizing amplified single-stranded nucleic acid fragments may also be used to measure the amount of a particular amplified single-stranded nucleic acid fragment in the amplification reaction mixture. For instance, in the embodiments where an amplified single nucleic acid molecule is first separated from the other molecules in the amplification reaction mixture by liquid chromatography and then subject to mass spectrometry analysis, the amount of the amplified single-stranded nucleic acid molecule may be quantified either by liquid chromatography of the fraction that contains the nucleic acid molecule, or by ion current measurement of the mass spectrometry peak corresponding to the nucleic acid molecule.

Such methodologies may be used to determine the allelic frequency of a target nucleic acid in a population of nucleic acids where the allelic variant(s) of the target nucleic acid may also be present. “Allelic variant” refers to a nucleic acid molecule that has an identical sequence to the target nucleic acid except at a defined location of the target nucleic acid. “Allelic frequency of a target nucleic acid in a population of nucleic acids” refers to the percentage of the total amount of the target nucleic acid and its allelic variant(s) in the nucleic acid population that is the target nucleic acid. Because the primer pairs used in preparing precursors to N1 are designed to anneal to portions of a target nucleic acid at each side of a potential genetic variation at a defined location in the target, the amplification using the primer pairs as primers and a nucleic acid population containing the target nucleic acid as templates produces the nucleic acid fragment that contains the genetic variation at the defined location of the target nucleic acid, as well as the nucleic acid fragment(s) that contains the genetic variations at the same location of the allelic variant(s) of the target nucleic acid if the variant(s) is present in the nucleic acid population. Because the sequences of the target nucleic acid and its allelic variant(s) differ only at the defined location, the precursors to N1 using the target nucleic acid and the allelic variant(s) as respective templates are amplified at an identical, or a similar, efficiency. Likewise, the single-stranded nucleic acid molecules (A1) that contain the genetic variation or its complement of the target nucleic acid are amplified at the efficiency identical or similar to that of the single-stranded nucleic acid molecules that contain the genetic variation or its complement of the allelic variants. In addition, if a T2 molecule is used that anneals to the A1 molecules amplified using the target and its allelic variants as respective templates at a same efficiency, the ratio of the A2 molecules amplified with the target as an initial template to the A2 molecules amplified using the variant(s) as an initial template reflects the ratio of the target to its variant(s) in the nucleic acid population. Thus, the measurement of the relative amount of A1 (or A2) molecules in the reaction mixture indicates the relative amount of the target nucleic acid in the nucleic acid population.

6. Compositions and Kits Useful in Genetic Variation Detection

Compositions and kits useful in genetic variation detection may be the same as those described above for exponential nucleic acid amplification. In certain embodiments, these kits may further comprise one or more additional components useful in characterizing amplification products. For instance, the additional component may be (1) a chromatography column; (2) a buffer for performing chromatographic characterization or separation of nucleic acids; (3) microtiter plates or microwell plates; (4) oligonucleotide standards (e.g., 6 mer, 7 mer, 8 mer, 10 mer, 12 mer, 14 mer and 16 mer) for liquid chromatography and/or mass spectrometry; and (5) an instruction booklet for using the kits.

7. Applications of the Present Genetic Variation Detection Methods

As described in detail above, the present invention provides methods for detecting and/or identifying genetic variations in target nucleic acids. Methods according to the present invention may find utility in a wide variety of applications where it is desirable or necessary to identify or measure genetic variations. Such applications include, but are not limited to, genetic analysis for hereditarily transferred diseases, tumor diagnosis, disease predisposition, forensics, paternity determination, enhancements in crop cultivation or animal breeding, expression profiling of cell function and/or disease marker genes, and identification and/or characterization of infectious organisms that cause infectious diseases in plants or animal and/or that are related to food safety.

For instance, the present invention may be useful in genetic analysis for forensic purposes. The identification of individuals at the level of DNA sequence variations is advantageous over conventional criteria such as fingerprints, blood type or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. The present invention is useful in characterizing polymorphism of sample DNAs, therefore useful in forensic DNA analysis. For example, the analysis of 22 separate gene sequences in a sample, each one present in two different forms in the population, could generate 1010 different outcomes, permitting the unique identification of human individuals.

The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such as point mutations (as in the c-K-ras oncogene in bladder carcinoma and in colorectal tumors), small deletions and small insertions. Each of the activation processes leads, in conjunction with additional degenerative processes, to an increased and uncontrolled cell growth. In addition, point mutations, small deletions or insertions may also inactivate the so-called “recessive oncogenes” and thereby leads to the formation of a tumor (as in the retinoblastoma (Rb) gene and the osteosarcoma). The present invention is useful in detecting or identifying the point mutations, small deletions and small mutations that activate oncogenes or inactivate recessive oncogenes, which in turn, cause cancers.

The present invention may also be useful in transplantation analyses. The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body.

The recognition of different foreign antigens is mediated by variable, antigen-specific regions of the T-cell receptor—analogous to the antibody reaction. In a graft rejection, the T-cells expressing a specific T-cell receptor that fits to the foreign antigen, could therefore be eliminated from the T-cell pool. Such analyses are possible by the identification of antigen-specific variable DNA sequences that are amplified by PCR and hence selectively increased. The specific amplification reaction permits the single cell-specific identification of a specific T-cell receptor.

Similar analyses are presently performed for the identification of auto-immune disease like juvenile diabetes, arteriosclerosis, multiple sclerosis, rheumatoid arthritis, or encephalomyelitis.

The present invention is useful for determining gene variations in T-cell receptor genes encoding variable, antigen-specific regions that are involved in the recognition of various foreign antigens. Thus, the present invention may be useful in predicting the probability of a rejection reaction of transplanted tissue.

The present invention is also useful in genome diagnostics. Four percent of all newborns are born with genetic defects; of the 3,500 hereditary diseases described which are caused by the modification of only a single gene, the primary molecular defects are only known for about 400 of them.

Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations.

Using direct and indirect DNA analysis, the diagnosis of a series of genetic diseases has become possible: bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. The present invention is useful in diagnosis of any genetic diseases that are caused by point mutations, small deletions or small insertions at defined positions.

In a related aspect, the present invention may be used in testing disease susceptibility. Certain gene variations, although they do not directly cause diseases, are associated to the diseases. In other words, the possession of the gene variations by a subject renders the subject susceptible to the diseases. The detection of such gene variations using the present methods enables the identification of the subjects that are susceptible to certain diseases and subsequent performance of preventive measures.

The present invention is also applicable to pharmocogenomics. For instance, it may be used to detect or identify genes that involve in drug tolerance, such as various alleles of cytochrome P450 gene.

In addition, the present invention provides methods useful for detecting or characterizing residual diseases. In other words, the present methods may be used for detecting or identifying remaining mutant genotypes as in cancer after certain treatments, such as surgery of chemotherapy. It may also useful in identifying emerging mutants, such as genetic variations in certain genes that render a pathogenic organism drug resistant.

### E. Use of Nucleic Acid Amplification Methods and Compositions in Pre-mRNA Alternative Splicing Analysis

The methods and compositions for exponential nucleic acid amplification may also be used for performing pre-mRNA alternative splicing analysis. A target cDNA or its portion that is suspected to contain a specific exon-exon junction is first incorporated into an initial nucleic acid molecule (N1) to be used as a template in a first amplification reaction. The initial nucleic acid molecule also comprises at least one strand of a first nicking agent recognition sequence and thus allows for the first amplification reaction in the presence of a DNA polymerase and a nicking agent that recognizes the first nicking agent recognition sequence. The product (A1) from the first amplification reaction comprises the portion of the target suspected to contain the specific exon-exon junction or its complementary portion. A1 then anneals to another template nucleic acid (T2). T2 comprises a sequence of the sense strand of a second nicking agent recognition sequence and thus allows for a second amplification reaction in the presence of the DNA polymerase and a nicking agent that recognizes the second nicking agent recognition sequence. The characterization of A1 and/or A2 indicates whether the target contains the specific exon-exon junction.

1. Definitions

An “exon” refers to any segment of an interrupted gene that is represented in the mature RNA product. An “intron” refers to a segment of DNA that is transcribed, but removed from within the transcript by splicing together the sequences (exons) on either side of it.

A “sense strand” of a cDNA molecule refers to the strand that has an identical sequence as the mRNA molecule from which the cDNA molecule is derived except that the nucleotide “U” in the mRNA is substituted by the nucleotide “T” in the cDNA molecule. An “antisense strand” of a cDNA molecule, on the other hand, refers to the strand that is complementary to the mRNA molecule from which the cDNA molecule is derived.

An exon (Exon A) is “upstream” to another exon (Exon B) in a same gene when the sequence of the sense strand of Exon A is 5′ to the sequence of the sense strand of Exon B. Exon A and Exon B may be further referred to as an upstream exon and a downstream exon, respectively.

A target cDNA molecule refers to a cDNA molecule that is derived from a gene of interest. In other words, it is the product of reverse transcription of an mRNA molecule resulting from the transcription of the gene of interest. The target cDNA molecule may have a partial sequence (i.e., reverse transcribed from a partial mRNA molecule), but preferably a full-length sequence.

A nucleic acid fragment encompassing a first ODNP and a second ODNP refers to a double-stranded nucleic acid fragment that one strand consists of the sequence of the first ODNP, the complementary sequence of the second ODNP, and the sequence between the first ODNP and the complementary sequence of the second ODNP; while the other strand consists of the complementary sequence of the first ODNP, the sequence of the second ODNP, and the sequence between the complementary sequence of the first ODNP and the sequence of the second ODNP.

“Differential splicing” or “alternative splicing” is the production of at least two different mRNA molecules from a same transcript of a gene. For instance, a particular segment of the transcript may be present in one of the mRNA molecules, but be spliced out from other mRNA molecules.

A “location suspected to be the junction of two specific exons” or a “location of a suspected junction of two specific exons” refers to the 3′ terminus of the sense strand of the relatively upstream exon and/or the 5′ terminus of the antisense strand of that exon.

A “junction of Exon A and Exon B” in a target cDNA refers to the location in the sense strand of the target cDNA where the 3′ terminus of Exon A is joined with the 5′ terminus of Exon B and/or the location in the antisense strand of the target cDNA where the 5′ terminus of Exon A is joined with the 3′ terminus of Exon B.

2. Initial Nucleic Acid Molecules (N1)

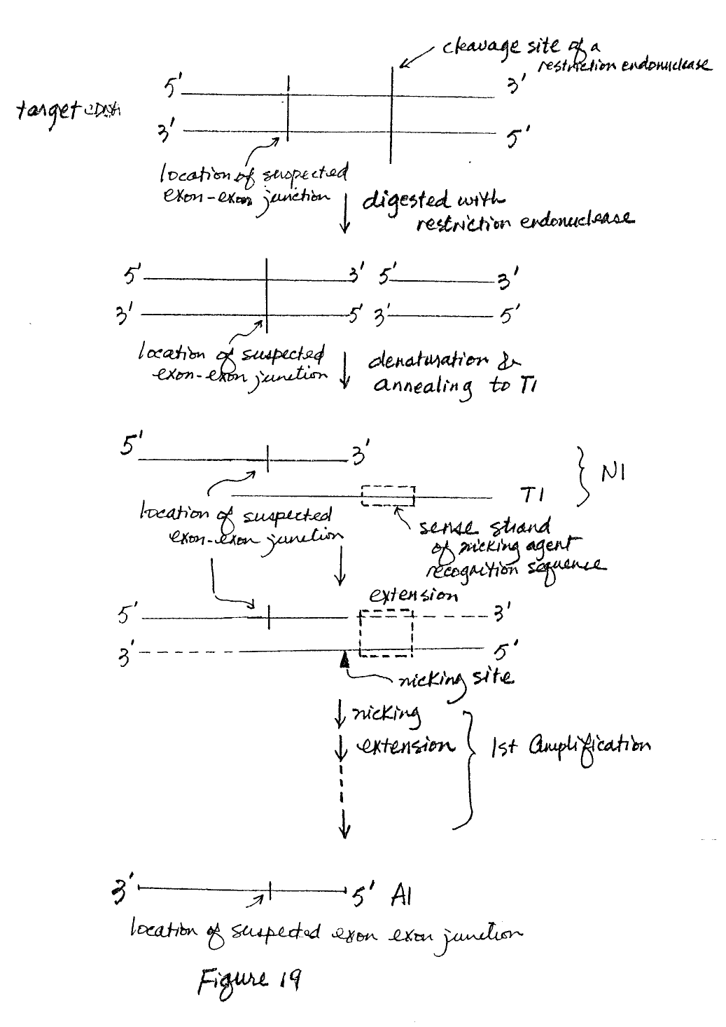

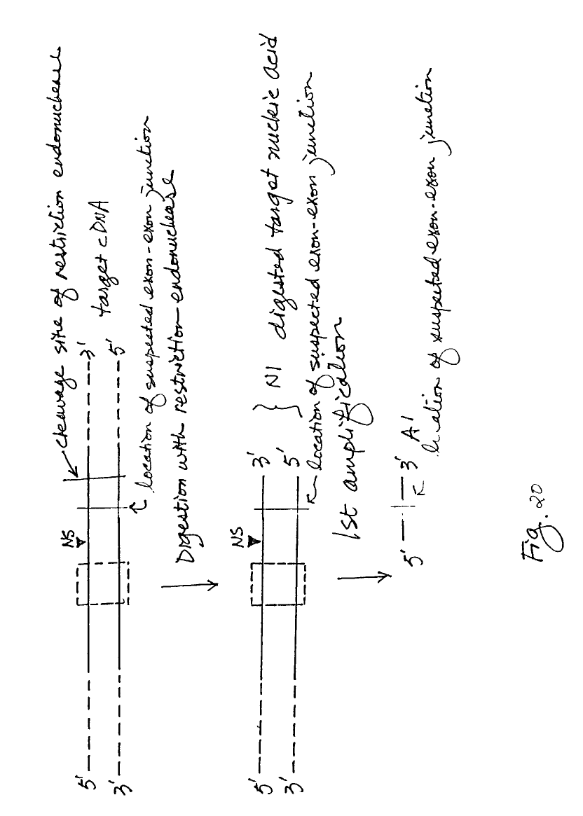

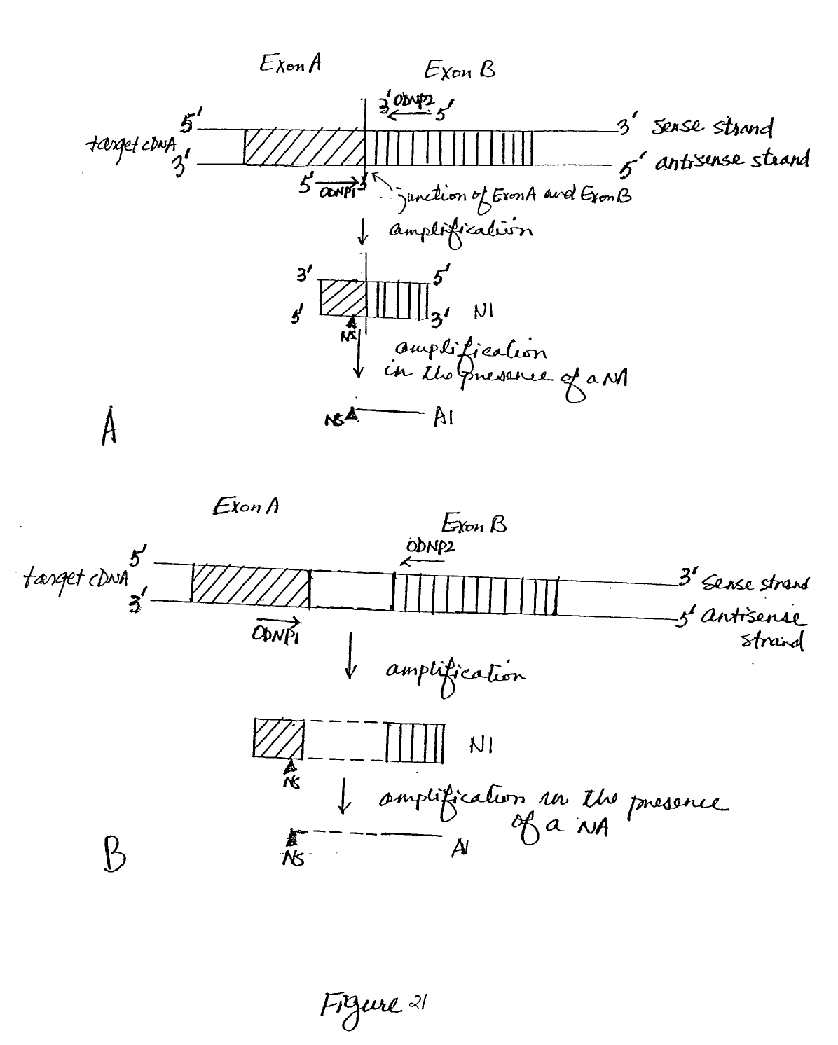

Initial nucleic acid molecules useful for differential splicing analysis may be provided by various approaches. For instance, N1 may be obtained by annealing of a trigger oligonucleotide primer to a T1 molecule where the trigger primer is derived from a target cDNA and encompasses the location suspected to be the junction of two exons (e.g., FIG. 19). Alternatively, N1 may be directly derived from a double-stranded target cDNA (e.g., by digestion of the target cDNA with a restriction endonuclease as shown in FIG. 20). N1 may also be prepared by the use of appropriate oligonucleotide primer pairs (e.g., FIGS. 21-24). Several exemplary means for providing initial nucleic acid molecules N1 are described below.

a. First Type of Exemplary Methods for Providing N1 Molecules

As noted above, N1 may be provided by annealing a trigger oligonucleotide primer to a T1 molecule. The trigger primer needs to encompass the location suspected to be a specific exon-exon junction. An example of this type of methods for providing N1 molecules is illustrated in FIG. 19. As shown in this figure, a double-stranded target cDNA is first cleaved by a restriction endonucelase whose recognition sequence is close to the location suspected to be a specific exon-exon junction. The digestion products may be denatured and the strand of the digestion product that contains the location suspected to be the specific exon-exon junction may then be used as a trigger oligonucleotide primer to anneal to a template nucleic acid (T1). T1 comprises a sequence of the sense strand of a nicking agent recognition sequence so that in the presence of a DNA polymerase and a nicking agent that recognizes the recognition sequence, a single-stranded nucleic acid fragment (A1) is amplified that contains the location suspected to be the specific exon-exon junction.

In certain embodiments, the target cDNA molecule may be immobilized to a solid support. In other embodiments, the T1 molecule may be immobilized, preferably via its 5′ terminus.

b. Second Type of Exemplary Methods for Providing N1 Molecules

In certain embodiments of the present invention, N1 is directly derived from a target cDNA that contains a location suspected to be a specific exon-exon junction and further comprises a nicking agent recognition sequence and a restriction endonucelase recognition sequence. An embodiment with a recognition sequence recognizable by a nicking endonuclease that nicks outside its recognition sequence (e.g., N.BstNB I) as an exemplary nicking agent recognition sequence is illustrated in FIG. 20. As shown in this figure, a target cDNA may be digested by a restriction endonuclease that recognizes a sequence in the target nucleic acid. The digestion product that contains the nicking endonuclease recognition sequence may function as an initial nucleic acid molecule (N1) to amplify a single-stranded nucleic acid fragment (A1). The location suspected to be a specific exon-exon junction needs to be between the nicking site produced by the nicking agent and the cleavage site of the restriction endonuclease so that the location is transferred or incorporated into the amplified A1 fragment.

c. Third Type of Exemplary Methods for Providing N1 Molecules

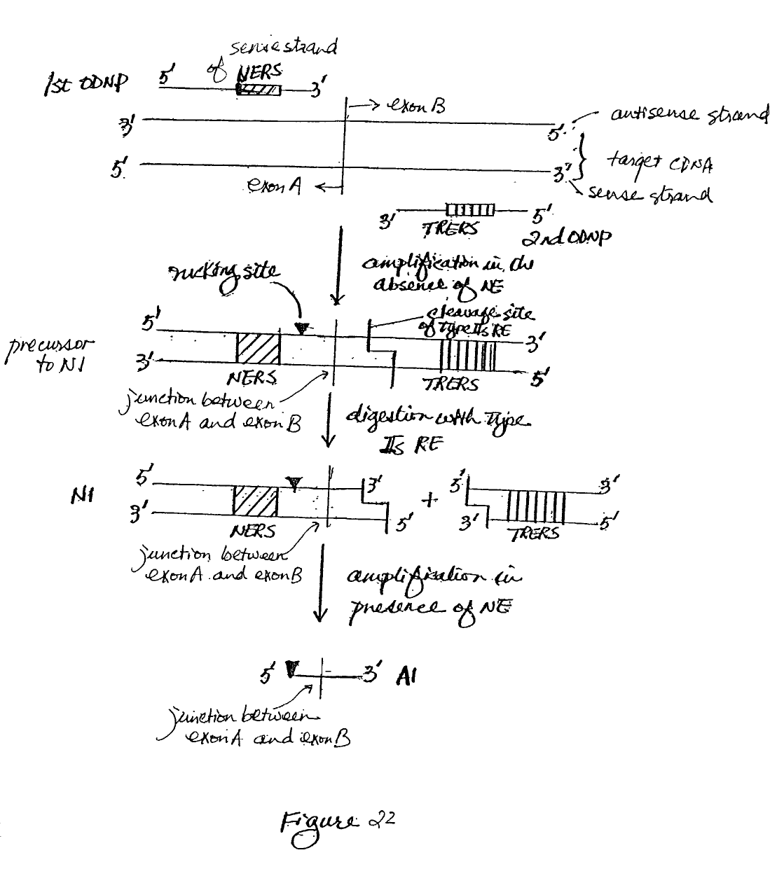

In certain embodiments, an initial nucleic acid molecule N1 is a completely or partially double-stranded nucleic acid molecule produced using various primer pairs. The following section first describes a general method for providing the above initial nucleic acid molecule (FIG. 21) and then provides certain specific embodiments of the general method (FIGS. 22-24).

For determining the presence or absence of a junction of an upstream exon (Exon A) and a downstream exon (Exon B), a primer pair composed of the following two primers may be used: (1) a first primer that comprises a sequence complementary to a portion of the antisense strand of Exon A near the 5′ terminus of Exon A in the antisense strand, and (2) a second primer that comprises a sequence complementary to a portion of the sense strand of Exon B near the 5′ terminus of Exon B in the sense strand (FIG. 21). The complementarity between the first ODNP and the portion of the antisense strand of Exon A needs not be exact, but must be sufficient to allow the ODNP to specifically anneal to that portion of Exon A. Likewise, the complementarity between the second ODNP and the portion of the sense strand of Exon B needs not be exact, but must be sufficient to allow the ODNP to specifically anneal to that portion of Exon B. A portion of a strand of an exon is near one of the termini of the exon if that portion is within 100, 90, 80, 70, 60, 50, 40, 35, 30, 25, 20, 15, or 10 nucleotides from that terminus in that strand. Such a spacing arrangement between the two ODNPs of the ODNP pair enables the amplification of a relatively short fragment encompassing the first and second primers using the target cDNA as a template if the junction of Exon A and Exon B is present in the target cDNA.

Besides the sequence complementarity between each primer and one strand of its corresponding exon, either the first or the second primer must further comprise a sequence of a sense strand of a nicking agent recognition sequence. The recognition sequencer may be recognizable by a nicking endonuclease or a restriction endonuclease. In certain preferred embodiments, both the first and second primers comprise a nicking agent recognition sequence. The presence of the recognition sequence allows the amplified nucleic acid fragments encompassing the first and second primers to function as a template nucleic acid for amplifying a single-stranded nucleic acid fragment (A1) in the presence of a DNA polymerase and a nicking agents that recognizes the recognition sequence.

When the primers and the target cDNA are combined in an amplification reaction, the presence (or absence) and composition of an amplification product reflects the presence or absence of the junction of Exon A and Exon B. If only Exon A or only Exon B is present in the target cDNA, no amplification product will be made using the above primers as primers and the target cDNA as a template. If both Exon A and Exon B are present in the target cDNA, an amplification product (i.e., a N1 molecule or a precursor to N1) will be made that encompasses the first and second primers. If the junction of Exon A and Exon B is present in the target cDNA, the amplification product will contain this junction (FIG. 21A). If the junction of Exon A and Exon B is absent (i.e., there is a sequence between Exon A and Exon B), the amplification product will not contain the junction but contain the sequence between the two exons (FIG. 21B). Thus, characterizing a single-stranded nucleic acid molecule (A1) amplified using N1 as a template and/or another single-stranded nucleic acid molecule (A2) using A1 as a template will indicate whether the target cDNA contains the junction of Exon A and Exon B.

A specific embodiment of the above general method is illustrated in FIG. 22. As indicated in this figure, the first primer comprises a sequence of the sense strand of a nicking endonuclease recognition sequence and anneals to a portion of the antisense strand of Exon A, whereas the second primer comprises a sequence of one strand of a type IIs restriction endonuclease recognition sequence and anneals to a portion of the sense strand of Exon B. When these two primers are used as primers to amplify a portion of the target cDNA, the amplification product (i.e., a precursor to N1) contains both strands of the nicking endonuclease recognition sequence and both strands of the type IIs restriction endonuclease recognition sequence. In addition, the amplification product also contains the junction of Exon A and Exon B if the junction is present in the target cDNA. In the presence of a type IIs restriction endonuclease that recognizes the type IIs restriction endonuclease recognition sequence, the amplification product is digested to produce a partially double-stranded nucleic acid molecule N1 that comprises both strands of the nicking endonuclease recognition sequence and also contains the junction of Exon A and Exon B if the junction is present in the target cDNA.

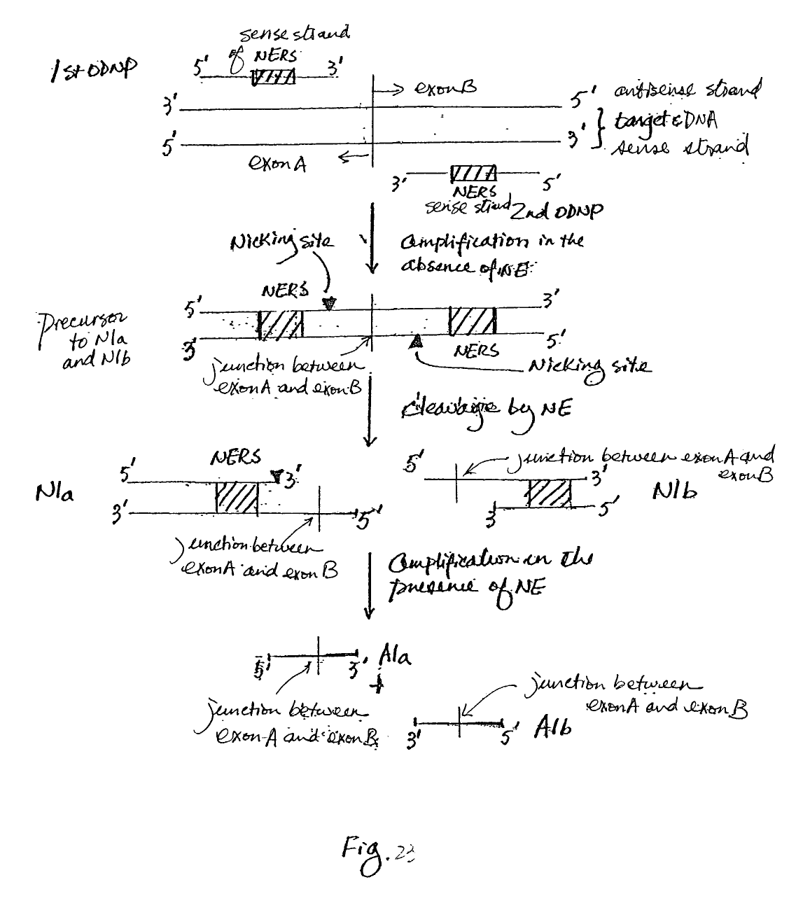

Another specific embodiment of the above general method is illustrated in FIG. 23. As indicated in this figure, both primers comprise a nicking endonuclease recognition sequence. In addition, the first primer is designed to anneal to a portion of the antisense strand of Exon A, whereas the second primer is designed to anneal to a portion of the sense strand of Exon B. When these two primers are used as primers to amplify a portion of the target cDNA, the amplification product (i.e., a precursor to N1) contains the junction of Exon A and Exon B if the junction is present in the target cDNA, as well as two double-stranded nicking endonuclease recognition sequences. These two recognition sequences may or may not be identical to each other, but preferably, they are identical. In the presence of a nicking endonuclease or nicking endonucleases that recognize the recognition sequences, the amplification product is nicked twice (once on each strand) to produce two partially double-stranded nucleic acid molecules (N1a and N1b) that each comprises one of the nicking endonuclease recognition sequences. In addition, the overhang of each of these two molecules also contains the junction of Exon A and Exon B if the junction is present in the target cDNA.

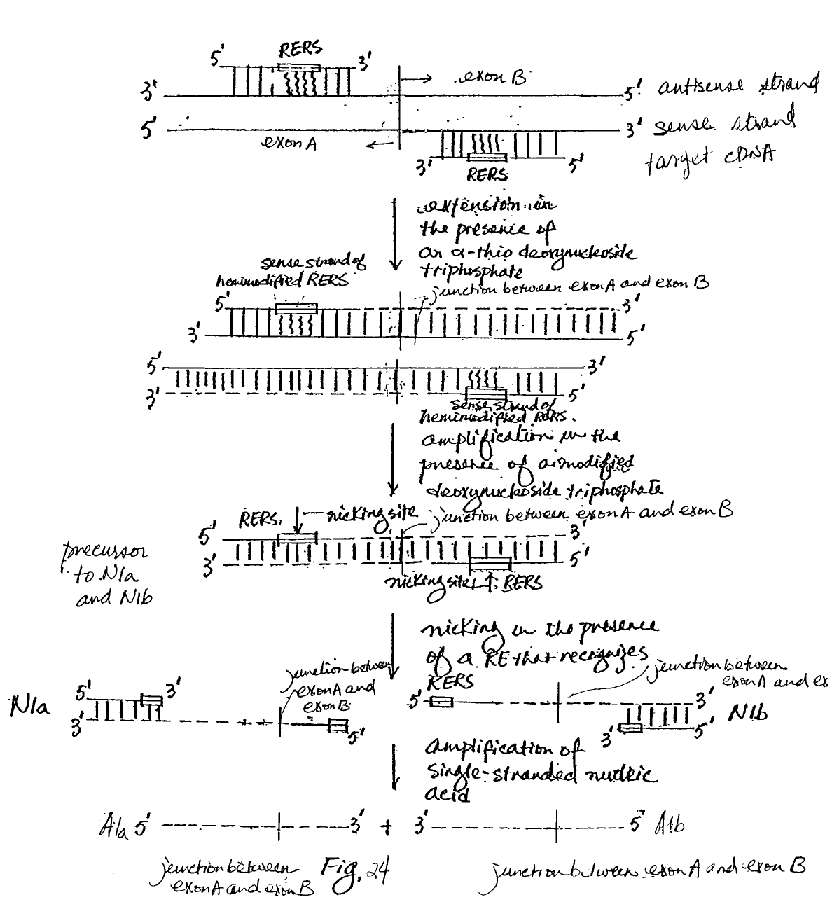

An additional specific embodiment of the above general method is illustrated in FIG. 24. As indicated in this figure, both primers comprise a restriction endonuclease recognition sequence. In addition, the first primer is designed to anneal to a portion of the antisense strand of Exon A, whereas the second primer is designed to anneal to a portion of the sense strand of Exon B. When these two primers are used as primers to amplify a portion of the target cDNA in the presence of a modified deoxynucleoside triphosphate, the amplification product (i.e., a precursor to N1) contains the junction of Exon A and Exon B if the junction is present in the target cDNA, as well as two hemimodified restriction endonuclease recognition sequences. These two hemimodified recognition sequences may or may not be identical to each other, but preferably, they are identical. In the presence of a restriction endonuclease or restriction endonucleases that recognize the recognition sequences, the amplification product is nicked twice (once on each strand) to produce two partially double-stranded nucleic acid molecules (N1a and N1b) that each comprises a sequence of one strand of one of the hemimodified recognition sequences. In addition, the overhang of each of these two molecules also contains the junction of Exon A and Exon B if the junction is present in the target cDNA.

The above first ODNP, the second ODNP or both may be immobilized to a solid support in certain embodiments. In other embodiments, the target cDNA molecule is immobilized.

3. A1 Molecules

As described above, an A1 molecule is amplified using a portion of N1 as a template. This portion of N1 comprises the location suspected to be a specific exon-exon junction so that this location is transferred or incorporated into A1. In certain embodiments, the length of A1 may be regulated to be relatively short in the case where the specific exon-exon junction is present in the target cDNA. For instance, for the third type of providing N1 molecules (FIGS. 21-24), the ODNP pair may be designed to be close to each other where they anneal to the target cDNA. More specifically, the first primer may be designed to anneal to a portion of the antisense strand of the target cDNA close to the 5′ terminus of Exon A, whereas the second primer may be designed to anneal to a portion of the sense strand of the target cDNA close to the 5′ terminus of Exon B. Similar to the diagnostic uses and genetic variation detection of the present invention described above, the short length of an A1 molecule increases amplification efficiencies and rates, allows for the use of a DNA polymerase that does not have a stand displacement activity, and facilitates the detection of A1 molecules and/or a product (A2) of a subsequent amplification reaction where A1 is used as an initial amplification primer via certain technologies such as mass spectrometric analysis.

4. T2 Molecules

A T2 molecule of the present invention comprises a sequence of the sense strand of a nicking agent recognition sequence as well as a sequence, located 3′ to the sequence of the sense strand of the recognition sequence, that is at least substantially complementary to a single-stranded nucleic acid molecule (A1) amplified using a portion of an initial nucleic acid molecule N1 as a template. Because T2 comprises a sequence of the sense strand of a nicking agent recognition sequence, in the presence of a nicking agent that recognizes the recognition sequence and a DNA polymerase, A1 is used as an initial amplification primer and subsequently used as a template for amplifying another single-stranded nucleic acid fragment (A2). As noted above, A1 contains the location suspected to be the specific exon-exon junction. This location is subsequently transferred or incorporated into A2. Accordingly, the characterization of A2 is able to determine the sequences at each side of the location and thus determine whether the specific exon-exon junction is present in the target cDNA.

Similar to the diagnostic application of the present invention, in certain embodiments of pre-mRNA alternative splicing analysis according to the present invention, no additional T2 molecules are needed for a second amplification reaction. In these embodiments, the second primer used in producing a N1 molecule has a 3′ terminal sequence that allows the second primer to anneal to A1. The second primer also comprises a sequence of the sense strand of a nicking agent recognition sequence. Thus, the extension of A1 using the second primer as a template creates a double-stranded nicking agent recognition sequence. In the presence of a DNA polymerase and a nicking agent that recognizes the recognition sequence, a single-stranded nucleic acid (A2) is amplified using A1 as a template.

A T2 molecule may be immobilized to a solid support, preferably via its 5′ terminus, in certain embodiments. In other embodiments, a T2 molecule may not be immobilized.

5. Characterizing Amplified Single-Stranded Nucleic Acids

The presence of a specific exon-exon junction in a target cDNA may be determined by characterizing an amplification product (i.e., A1 or A2). Any method suitable for characterizing single-stranded nucleic acid molecules may be used. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445.

The characteristics of the amplified single-stranded nucleic acid fragments (e.g., the mass to charge ratio obtained by mass spectrometric analysis) are subsequently compared with those of single-stranded nucleic acid fragments predicted in view of the positions and compositions of the primers used in preparing template nucleic acid fragments and with the assumption that the junction between the two exons to which the primers are complementary is present. If the characteristics of the amplified and the predicted nucleic acid fragments are identical, the particular exon-exon junction that was assumed to be present in the target cDNA molecule is in fact present in that target cDNA molecule. The prediction of the sequence and the characteristics (e.g., mass to charge ratio) of the single-stranded nucleic acid fragment that would be amplified is based on the knowledge about consensus sequences near exon-intron junctions. This knowledge allows one of ordinary skill in the art to pinpoint the exon-intron junctions and thus predicts the exact locations of exon-exon junctions when the intron between the two exons has been spliced out.

6. Compositions and Kits Useful in Pre-mRNA Differential Splicing Analysis

Compositions and kits useful in pre-mRNA differential splicing analysis may be the same as those described above for exponential nucleic acid amplification. In certain embodiments, these kits may further comprise one or more additional components useful in characterizing amplification products. For instance, the additional component may be (1) a chromatography column; (2) a buffer for performing chromatographic characterization or separation of nucleic acids; (3) microtiter plates or microwell plates; (4) oligonucleotide standards (e.g., 6 mer, 7 mer, 8 mer, 10 mer, 12 mer, 14 mer and 16 mer) for liquid chromatography and/or mass spectrometry; (5) a reverse transcriptase; (6) a buffer for a reverse transcriptase, and (7) an instruction booklet for using the kits.

7. Applications of the Present Pre-mRNA Differential Splicing Analysis

The present invention is useful in detecting any mRNA differential splicing of interest. Alternative pre-mRNA splicing is an important mechanism for regulating gene expression in higher eukaryotes. By recent estimates, the primary transcripts of ˜30% of human genes are subject to alternative splicing, often regulated in specific spatial/temporal patterns during normal development. In complex genes alternative splicing can generate dozens or even hundreds of different mRNA isoforms from a single transcript (Breitbart and Nadal-Ginard, Annu. Rev. Biochem. 56: 467-95, 1987; Missler and Sudhof, Trends Genet 14: 20-6, 1998; Gascard et al., Blood 92:4404-14, 1998). In many cases the alternatively spliced exon encodes a protein domain that is functionally important for catalytic activity or binding interactions, the resulting proteins can exhibit different or even antagonistic activities.

As discussed in detail herein above, the present invention provides methods, compositions, and kits for detecting pre-mRNA alternative splicing, including the detection of alternative splicing at a terminus of a particular exon of a gene in a cDNA molecule or a cDNA population, and at every terminus of every exon of a gene in a cDNA molecule or a cDNA population. Due to the importance of pre-mRNA splicing, these methods, compositions and kits will find utility in a wide variety of applications such as disease diagnosis, predisposition, and treatment, crop cultivation and animal breeding, development regulations of plants and animals, drug development and manipulation of responses of an organism to external stimuli (e.g., extreme temperatures, poison, and light).

For instance, the present method may be used to identify and/or characterize pre-mRNA splicing patterns unique to a pathological condition. Abnormal pre-mRNA splicings in many genes have been implicated in various diseases or disorders, especially in cancers. In small cell lung carcinoma, the gene of protein p130, which belongs to the retinoblastoma protein family is mutated at a consensus splicing site. This mutation results in the removal of exon 2 and the absence of synthesis of the protein due to the presence of a premature stop codon. Likewise, in certain non small cell lung cancers, the gene of protein p161NK4A, which is an inhibitor of cyclin dependant kinase cdk4 and cdk6, is mutated at a donor splicing site. This mutation results in the production of a truncated short half-life protein. In addition, WT1, the Wilm's tumor suppressor gene, is transcribed into several messenger RNAs generated by alternative splicings. In breast cancers, the relative proportions of different variants are modified in comparison to healthy tissue, hence yielding diagnostic tools or insights into understanding the importance of the various functional domains of WT1 in tumoral progression. A similar alteration process affecting ratios among different mRNA forms and protein isoforms during cell transformation is also found in neurofibrin NF1. Moreover, in head and neck cancer, one of the mechanisms by which p53 is inactivated involved a mutation at a consensus splicing site. Furthermore, an altered splicing pattern of the IRF-1 tumor suppressor gene transcript results in the inactivation of the tumor suppressor and an acceleration of exon skipping in IRF-1 mRNA is indicative of a number of hematopoietic disorders including overt leukemia from myelodysplastic syndrome, acute myeloid leukemia, and the myelodysplastic syndromes (U.S. Pat. No. 5,643,729).

The present method may be used to compare the splicing pattern of the transcript of a gene that is known or suspected to be associated with a disease (or disorder) condition, and to identify exons of which presence or absence is unique to the disease (or disorder) condition or to identify the alteration in the ratio among different splicing variants unique to the disease (or disorder) condition. The identification of the exons that are absence in a disease (or disorder) condition may indicate that the domains encoded by the exons are important to the normal functions of healthy cells and that the signaling pathways involving such domains may be restored for therapeutical purposes. On the other hand, the identification of the exons uniquely present in a disease (or disorder) condition may be used as diagnostic tools and the domains encoded thereof be considered as screening targets for compounds of low molecular weight intended to antagonize signal transduction mediated by the domains. In addition, the antibodies with specific affinities to these domains may also be used as diagnostic tools for the disease (or disorder) condition.

The present method may also be used to identify and/or characterize the pre-mRNA differential splicing important in organism development. Alternative splicing plays a major role in sex determination in Drosophila, antibody response in humans and other tissue or developmental stage specific processes (Chabot, Trends Genet. 12: 472-8; Smith et al., Annu. Rev. Genet. 23: 527-77, 1989; Breitbart et al., Cell 49: 793-803, 1987). Thus, the present method may be used to compare pre-mRNA splicing patterns of a gene that is known or suspected to be involved in development regulation at different developmental stages. The identification and/or characterization of the presence of differential splicing in the gene may provide guidance in regulating the corresponding development process to obtain desirable traits (e.g., bigger fruits, higher protein or oil content seeds, higher milk production).

The present method may also be used to identify and/or characterize the pre-mRNA differential splicing important in organisms' responses to various external stimuli. The pre-mRNA splicing pattern of a gene that is known or suspected to play a role in response to a particular stimulus (e.g., pathogen attack) of an untreated organism may be compared with that of an organism subjected to the stimulus. The identification and/or characterization of the splicing pattern unique to the organism subjected to the stimulus may provide guidance in manipulating the corresponding response process to enhance (if the response is desirable) or to reduce/eliminate (if the response is undesirable) the response.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for amplifying a nucleic acid molecule (A2), comprising:
(A) providing an at least partially double-stranded nucleic acid molecule (N1) comprising at least one of
(i) a sequence of the sense strand of a first nicking agent recognition sequence (NARS), and
(ii) a sequence of the antisense strand of the first NARS;
(B) amplifying a first single-stranded nucleic acid molecule (A1) in the presence of a nicking agent (NA) that recognizes the first NARS, a DNA polymerase, and one or more deoxynucleoside triphosphate(s), wherein the amplifying uses a portion of N1 as a template for the polymerase;
(C) providing a second single-stranded nucleic acid molecule (T2) comprising, from 5′ to 3′:
(i) a sequence of the sense strand of a second NARS, and
(ii) a sequence that is at least substantially complementary to A1; and
(D) amplifying a third single-stranded nucleic acid molecule (A2) in the presence of T2, A1, the first NA, a second NA that recognizes the second NARS, the DNA polymerase, and the deoxynucleoside triphosphate(s), wherein A2 is at least substantially complementary to A1 and wherein A1, A2 or both are at most 25 nucleotides in length.

2. The method of claim 1 wherein the first NARS is identical to the second NARS.

3. The method of claim 2 wherein the first and second NARS are a nicking endonuclease.

4. The method of claim 1 wherein both the first and the second NAs are a nicking endonuclease (NE).

5. The method of claim 1 wherein steps (A)-(D) are performed in a single vessel.

6. The method of claim 1 wherein the N1 is immobilized.

7. The method of claim 1 wherein the T2 is immobilized.

8. The method of claim 1 wherein N1 comprises the sequence of the antisense strand of the first NARS.

9. The method of claim 1 wherein N1 comprises the sequence of the sense strand of the first NARS.

10. The method of claim 9 wherein both the first and the second NAs are restriction endonucleases (REs) and at least one of the nucleoside triphosphate(s) is modified.

11. The method of claim 1 wherein N1 is provided by annealing a trigger oligonucleotide primer (ODNP) to a single-stranded nucleic acid molecule (T1) comprising the sequence of the sense or the antisense strand of the first NARS.

12. The method of claim 1 wherein A1 is from 8 to 24 nucleotides in length.

13. The method of claim 12 wherein A1 is from 12 to 17 nucleotides in length.

14. The method of claim 1 wherein A2 is from 8 to 24 nucleotides in length.

15. The method of claim 14 wherein A2 is from 12 to 17 nucleotides in length.

16. The method of claim 1 wherein the initial number of T2 is more than that of T1.

17. The method of claim 1 wherein N1 is derived from a genomic DNA.

18. The method of claim 1 wherein N1 is a portion of a genomic DNA.

19. A method for amplifying a nucleic acid molecule (A2), comprising:
(A) forming a mixture comprising:
(i) an at least partially double-stranded nucleic acid molecule (N1) comprising a sequence of an antisense strand of a first nicking agent recognition sequence (NARS);
(ii) a single-stranded nucleic acid molecule (T2) comprising, from 3′ to 5′:
(a) a sequence that is at least substantially identical to a portion of N1 located 5′ to the sequence of the antisense strand of the first NARS in N1; and
(b) a sequence of a sense strand of a second NERS; and
(iii) a first nicking agent (NA) that recognizes the first NARS, a second NA that recognizes the second NARS, a DNA polymerase, and one or more deoxynucleoside triphosphate(s); and
(B) maintaining said mixture at conditions that exponentially amplify a single-stranded nucleic acid molecule (A2), wherein A2 is at most 25 nucleotides in length.

20. The method of claim 19 wherein the first and second NAs are a nicking endonuclease (NE).

21. The method of claim 20 wherein the first and second NAs are identical.

22. The method of claim 19 wherein the N1 is immobilized.

23. The method of claim 19 wherein the T2 is immobilized.

24. The method of claim 19 wherein sequence (A)(ii)(a) is exactly identical to a portion of N1 located 5′ to the antisense strand of the first NARS in N1.

25. The method of claim 19 wherein N1 is provided by annealing a trigger oligonucleotide primer (ODNP) to a single-stranded target nucleic acid (T1), where T1 comprises, from 5′ to 3′:
(A) a sequence of an antisense strand of the first NARS; and
(B) a sequence, located 3′ to the sequence of the antisense strand of the first NARS, that is at least substantially complementary to at least a portion of the trigger ODNP.

26. The method of claim 25 wherein the T1 is immobilized.

27. The method of claim 25 wherein the sequence (B) of T1 is exactly complementary to at least a portion of the trigger ODNP.

28. The method of claim 25 wherein the 3′ terminus of T1 is linked to a phosphate group.

29. The method of claim 19 wherein the sequence 5′ to the sequence of the antisense strand of the first NARS in N1 is derived from a target nucleic acid molecule and contains a genetic variation.

30. The method of claim 29 further comprising characterizing A2 to identify the genetic variation in the target nucleic acid molecule.

31. The method of claim 19 wherein the sequence 5′ to the sequence of the antisense strand of the first NARS in N1 is derived from a cDNA molecule and is suspected to contain a junction between two specific exons.

32. The method of claim 31 further comprising characterizing A2 to determine whether the cDNA contains the junction between the two exons.

33. The method of claim 30 or claim 32 wherein the characterizing is performed at least partially by the use of a technique selected from the group consisting of mass spectrometry, liquid chromatography, fluorescence polarization, and electrophoresis.

34. The method of claim 33 wherein the characterizing is performed at least partially by the use of liquid chromatography.

35. The method of claim 33 wherein the characterizing is performed at least partially by the use of mass spectrometry.

36. A method for amplifying a nucleic acid molecule (A2), comprising:
(A) forming a mixture of
(i) an at least partially double-stranded nucleic acid molecule (N1) comprising a sequence of a sense strand of a first nicking agent recognition sequence (NARS);
(ii) a single-stranded nucleic acid molecule (T2) comprising, from 3′ to 5′:
(a) a sequence that is at least substantially complementary to a portion of N1 located 3′ to the sense strand of the first NARS in N1, and
(b) a sequence of a sense strand of a second NARS; and
(iii) a first nicking agent (NA) that recognizes the first NARS, a second NA that recognizes the second NARS; a DNA polymerase; and one or more deoxynucleoside triphosphate(s); and
(B) maintaining said mixture at conditions that amplify a single-stranded nucleic acid molecule (A2), wherein A2 is at most 25 nucleotides in length.

37. The method of claim 36 wherein both the first and the second NAs are nicking endonucleases (NEs).

38. The method of claim 37 wherein the first and second NAs are identical.

39. The method of claim 36 wherein both the first and the second NAs are restriction endonucleases (REs), and at least one of the deoxynucleoside triphosphate(s) is modified.

40. The method of claim 36 wherein the N1 is immobilized.

41. The method of claim 36 wherein the T2 is immobilized.

42. The method of claim 36 wherein sequence (ii)(a) is exactly complementary to a portion of N1 located 3′ to the sense strand of the first NARS.

43. The method of claim 36 wherein N1 is provided by annealing a trigger oligonucleotide (ODNP) to a single-stranded target nucleic acid (T1) that comprises, from 5′ to 3′:
(A) a sequence of a sense strand of the first NARS; and
(B) a sequence that is at least substantially complementary to at least a portion of the trigger ODNP.

44. The method of claim 43 wherein the trigger ODNP is derived from a target nucleic acid and contains a genetic variation of the target nucleic acid.

45. The method of claim 44 further comprising characterizing A2 to identify the genetic variation in the target nucleic acid.

46. The method of claim 43 wherein the trigger ODNP is derived from a cDNA molecule and is suspected to contain a junction between two specific exons

47. The method of claim 46 further comprising characterizing A2 to determine whether the cDNA contains the junction between the two exons.

48. The method of claim 43 wherein the T1 is immobilized.

49. The method of claim 43 wherein sequence (B) of T1 is exactly complementary to at least a portion of the trigger ODNP.

50. A tandem nucleic acid amplification system comprising a first primer extension means for amplifying a first nucleic acid (A1) and a second primer extension means for amplifying a second nucleic acid (A2), wherein
(i) A1 is the initial primer for the second primer extension means for amplifying A2;
(ii) both the first and second primer extension means are contained within a single reaction vessel and require the presence of a nicking agent (NA);
(iii) A1, A2 or both are at most 25 nucleotides in length; and
(iv) A2 is at least substantially complementary to A1.

51. The tandem nucleic acid amplification system of claim 50 wherein the NA for the first primer extension means is identical to the NA for the second primer extension means.

52. The tandem nucleic acid amplification system of claim 50, wherein
(a) the first means for amplifying A1 comprises a first oligonucleotide primer (trigger ODNP), a first template nucleic acid (T1) at least substantially complementary to the trigger ODNP, a first nicking agent (NA), a first DNA polymerase, wherein the extension of the trigger ODNP using T1 as a template produces a first nicking agent recognition sequence (NARS) that is recognizable by the first NA; and
(b) the second means for amplifying A2 comprises the nucleic acid (A1), a second template nucleic acid (T2) at least substantially complementary to A1, and comprising a sequence of the sense strand of a second NARS, a second NA that recognizes the second NARS, and a second DNA polymerase.

53. The method of claim 52 wherein the first NA is identical to the second NA.

54. The method of claim 52 or 53 wherein the first polymerase is identical to the second polymerase.

55. The nucleic acid amplification system of claim 52 wherein both the first NA and the second NAs are nicking endonucleases.

56. The nucleic acid amplification system of claim 52 wherein both the first and the second NAs are restriction endonucleases.

57. The nucleic acid amplification system of claim 52 wherein the trigger ODNP is immobilized.

58. The nucleic acid amplification system of claim 52 wherein the T1 is immobilized.

59. The nucleic acid amplification system of claim 52 wherein the T2 is immobilized.

60. A method for exponential amplification of a nucleic acid molecule A2 comprising
(a) amplifying a nucleic acid molecule (A1) using a first template nucleic acid (T1) comprising the sequence of one strand of a first nicking agent recognition sequence (NARS) as a template by a primer extension reaction in the presence of a first nicking endonuclease (NA) that recognizes the first NARS and a first DNA polymerase; and
(b) amplifying A2 using a second template nucleic acid (T2) comprising the sequence of the sense strand of a second NARS as a template and A1 as the initial primer by a primer extension reaction in the presence of a second NA and a second DNA polymerase;
wherein A1, A2 or both are at most 25 nucleotides in length.

61. The method of claim 60 wherein the first NARS is identical to the second NARS.

62. The method of claim 60 or claim 61 wherein the first DNA polymerase is identical to the second DNA polymerase.

63. The method of claim 60 wherein steps (a) and (b) are performed in a single vessel.

64. The method of claim 61 wherein the NA is a nicking endonuclease (NE).

65. The method of claim 61 wherein the NA is a restriction endonuclease (RE).

66. The method of claim 60 wherein the T1 is immobilized.

67. The method of claim 60 wherein the T2 is immobilized.

68. The method of any one of claims 3, 20 and 37 wherein the NE is N.BstNB I or N.Alw I.

69. The method of claim 68 wherein the both the first and the second NEs are N.BstNB I.

70. The method of any one of claims 1, 19 and 36 wherein the amplification is performed under isothermal conditions.

71. The method of claim 70 wherein each amplification reaction is performed at 50° C.-70° C.

72. The method of any one of claims 1, 19 and 36 wherein the DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, and T4 DNA polymerase homoenzyme.

73. The method of claim 74 wherein the 5′→3′ exonuclease deficient DNA polymerase is exo⁻ Bst polymerase, exo⁻ Bca polymerase, exo⁻ Vent polymerase, 9° Nm™ DNA polymerasen, or exo⁻ Deep Vent polymerase.

74. A composition comprising:
(A) a first at least partially double-stranded nucleic acid molecule of which one strand comprises from 5′ to 3′:
(i) a sequence at most 25 nucleotides in length, and
(ii) a sequence of the antisense strand of a first nicking agent recognition sequence (NARS); and
(B) a second at least double-stranded nucleic acid molecule of which one strand comprises, from 5′ to 3′:
(i) a sequence of the sense strand of a second NARS, and
(ii) a sequence at least substantially identical to a sequence located 5′ to the sequence of the antisense strand of the first NARS in the first nucleic acid molecule.

75. The composition of claim 74 wherein the first NARS is recognizable by a first nicking endonuclease (NE), and the second NARS is recognizable by a second NE.

76. The composition of claim 74 wherein the first NARS is recognizable by a restriction endonuclease and the second NARS is recognizable by a nicking endonuclease.

77. The composition of claim 74 wherein the first at least partially double-stranded nucleic acid molecule is immobilized.

78. The composition of claim 74 wherein the second at least partially double-stranded nucleic acid molecule is immobilized.

79. The composition of claim 74 wherein sequence (B) (ii) is exactly identical to a sequence located 5′ to the sequence of the antisense strand of the first NERS in the first nucleic acid molecule.

80. The composition of claim 74 wherein the first NARS is identical to the second NARS.

81. The composition of claim 80 wherein the first and second NARS is recognizable by a NE.

82. A composition comprising:
(a) a first at least partially double-stranded nucleic acid molecule of which one strand comprises a sequence of the sense strand of a first nicking agent recognition sequence (NARS); and
(b) a second at least double-stranded nucleic acid molecule of which one strand comprises from 5′ to 3′:
(i) a sequence of the sense strand of a second NARS, and
(ii) a sequence that is at least substantially complementary to a sequence located 3′ to the sequence of the sense strand of the first NARS in the first nucleic acid molecule,
wherein in the presence of a nicking agent that recognizes the first NARS, a DNA polymerase, and one or more nucleoside triphospates, a single-stranded nucleic acid fragment amplified using the first nucleic acid molecule as a template has at most 25 nucleotides.

83. The composition of claim 82 wherein the first NARS is recognizable by a first nicking endonuclease (NE), and the second NARS is recognizable by a second NE.

84. The composition of claim 82 wherein the first NARS is recognizable by a first restriction endonuclease (RE), and the second NARS is recognizable by a second RE.

85. The composition of claim 82 wherein the first at least partially double-stranded nucleic acid molecule is immobilized.

86. The composition of claim 85 wherein the second at least partially double-stranded nucleic acid molecule is immobilized.

87. The composition of claim 82 wherein sequence (b) (ii) is exactly complementary to a sequence located 3′ to the sequence of the sense strand of the first NARS in the first nucleic acid molecule.

88. The composition of claim 82 wherein the first NARS is identical to the second NARS.

89. The composition of claim 88 wherein the first and second NARSs are recognizable by a NE.

90. The composition of claim 72 or claim 82 further comprising a first NA that recognizes the first NARS and a second NA that recognizes the second NARS.

91. The composition of claim 80 or claim 88 further comprising a NA that recognizes both the first and second NARSs.

92. The composition of claim 81 or claim 89 further comprising a NE that recognizes both the first and second NARSs.

93. The composition of claim 93 wherein the NE is N.BstNB I.

94. The composition of any one of claims 74, 82 and 90 further comprising a DNA polymerase.

95. The composition of claim 94 wherein the DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polyamerase and T4 DNA polymerase homoenzyme.

96. The composition of claim 94 wherein the 5′→3′ exonuclease deficient DNA polymerase is exo⁻ Bst polymerase, exo⁻ Bca polymerase, exo⁻ Vent polymerase, exo⁻ Deep Vent polymerase, or 9° Nm™ DNA polymerase.

97. A method for identifying a gene variation in a genomic nucleic acid or cDNA molecule, wherein the genetic variation is located 5′ to a sequence of the antisense strand of a first nicking endonuclease recognition sequence (NERS) in the genomic nucleic acid or cDNA molecule, the method comprising:
(A) forming a mixture comprising:
(i) the genomic nucleic acid or cDNA molecule,
(ii) a single-stranded nucleic acid molecule (T2) comprising from 3′ to 5′:
(a) a sequence that is at least substantially identical to a portion of the genomic nucleic acid or cDNA molecule located 5′ to the sequence of the antisense strand of the first NERS, and
(b) a sequence of the sense strand of a second NERS, and
(iii) a first nicking endonuclease (NE) that recognizes the first NERS; a second NE that recognizes the second NERS, a DNA polymerase, and one or more deoxynucleoside triphosphate(s);
(B) maintaining the mixture at conditions that exponentially amplify a single-stranded nucleic acid molecule (A2); and
(C) characterizing A2 to identify the gene variation in the genomic nucleic acid or cDNA molecule.

98. The method of claim 97 wherein the first NERS is identical to the second NERS.

99. A method for identifying a genetic variation at a defined location in a target nucleic acid, comprising
(a) forming a mixture of a first oligonucleotide primer (ODNP), a second ODNP, and the target nucleic acid, wherein
(i) if the target nucleic acid is a double-stranded nucleic acid having a first strand and a second strand, then
the first ODNP comprises a nucleotide sequence of a sense strand of a first nicking endonuclease recognition sequence (NERS) and a nucleotide sequence at least substantially complementary to a nucleotide sequence of the first strand of the target nucleic acid located 3′ to the complement of the genetic variation, and
the second ODNP comprises a nucleotide sequence at least substantially complementary to a nucleotide sequence of the second strand of the target nucleic acid located 3′ to the genetic variation and optionally comprises a sequence of one strand of a restriction endonuclease recognition sequence (RERS),
or
(ii) if the target nucleic acid is a single-stranded nucleic acid, then
the first ODNP comprises a nucleotide sequence of a sense strand of a first NERS and a nucleotide sequence at least substantially identical to a nucleotide sequence of the target nucleic acid located 5′ to the genetic variation, and
the second ODNP comprises a nucleotide sequence at least substantially complementary to a nucleotide sequence of the target nucleic acid located 3′ to the genetic variation and optionally comprises a RERS; and
(b) extending the first and the second ODNPs to produce an extension product encomprising the first ODNP and the second ODNP;
(c) optionally digesting the extension product of step (b) with a restriction endonuclease that recognizes the RERS to produce a digestion product;
(d) amplifying a first single-stranded nucleic acid fragment (A1) using one strand of the extension product of step (b) or the digestion product of step (c) as a template in the presence of a nicking endonuclease (NE) that recognizes the first NERS;
(e) providing a second single-stranded nucleic acid molecule (T2) to anneal to A1, T2 comprising, from 5′ to 3′:
(i) a sequence of the sense strand of a second NERS, and
(ii) a sequence at least substantially complementary to A1;
(f) amplifying a third single-stranded nucleic acid fragment (A2) using A1 as a template; and
(e) characterizing A2 to identify the genetic variation in the target nucleic acid,
wherein A1, A2 or both have at most 25 nucleotides.

100. The method of claim 99 wherein the first NERS is identical to the second NERS.

101. A method for identifying a genetic variation at a defined location in a target nucleic acid, comprising:
(a) forming a mixture of a first ODNP, a second ODNP, and the target nucleic acid, wherein
(i) if the target nucleic acid is a double-stranded nucleic acid having a first strand and a second strand, then
the first ODNP comprises a nucleotide sequence of one strand of a first restriction endonuclease recognition sequence (RERS) and a nucleotide sequence at least substantially complementary to a nucleotide sequence of the first strand of the target nucleic acid located 3′ to the complement of the genetic variation, and
the second ODNP comprises a nucleotide sequence of one strand of a second RERS and a nucleotide sequence at least substantially complementary to a nucleotide sequence of the second strand of the target nucleic acid located 3′ to the genetic variation;
or
(ii) if the target nucleic acid is a single-stranded nucleic acid, then
the first ODNP comprises a nucleotide sequence of one strand of a first RERS and a nucleotide sequence at least substantially identical to a nucleotide sequence of the target nucleic acid located 5′ to the complement of the genetic variation, and
the second ODNP comprises a sequence of one strand of a second RERS and a nucleotide sequence at least substantially complementary to a nucleotide sequence of the target nucleic acid located 3′ to the genetic variation;
(b) extending the first and the second ODNPs in the presence of deoxyribonucleoside triphosphates and at least one modified deoxyribonucleoside triphosphate to produce an extension product comprising both the first and the second RERSs;
(c) exponentially amplifying single-stranded nucleic acid fragments using the extension product of step (b) as a template in the presence of restriction endonucleases (REs) that recognize the first RERS and the second RERS, wherein the single-stranded nucleic acid fragment is no more than 25 nucleotides in length; and
(d) characterizing at least one of the single-stranded fragments of step (c) to identify the genetic variation.

102. The method of claim 101 wherein the first RERS is identical to the second RERS.

103. The method of claim 101 wherein the first ODNP, the second ODNP or both ODNPs are immobilized.

104. The method of claim 101 wherein the target nucleic acid is immobilized.

105. A method for identifying a genetic variation at a defined location in a target nucleic acid, comprising:
(a) forming a mixture of a first ODNP, a second ODNP, and the target nucleic acid, wherein
(i) if the target nucleic acid is a double-stranded nucleic acid having a first strand and a second strand, then
the first ODNP comprises a nucleotide sequence of one strand of a first restriction endonuclease recognition sequence (RERS) and a nucleotide sequence at least substantially complementary to a nucleotide sequence of the first strand of the target nucleic acid located 3′ to the complement of the genetic variation, and
the second ODNP comprises a nucleotide sequence of one strand of a second RERS and a nucleotide sequence at least substantially complementary to a nucleotide sequence of the second strand of the target nucleic acid located 3′ to the genetic variation;
or
(ii) if the target nucleic acid is a single-stranded nucleic acid, then
the first ODNP comprises a nucleotide sequence of one strand of a first RERS and a nucleotide sequence at least substantially identical to a nucleotide sequence of the target nucleic acid located 5′ to the complement of the genetic variation, and
the second ODNP comprises a sequence of one strand of a second RERS and a nucleotide sequence at least substantially complementary to a nucleotide sequence of the target nucleic acid located 3′ to the genetic variation;
(b) extending the first and the second ODNPs in the presence of deoxyribonucleoside triphosphates and at least one modified deoxyribonucleoside triphosphate to produce an extension product comprising both the first and the second RERSs;
(c) amplifying a first single-stranded nucleic acid fragment using one strand of the extension product of step (b) as a template in the presence of restriction endonucleases (REs) that recognize the first RERS and the second RERS;
(d) providing a second single-stranded nucleic acid molecule (T2) to anneal to A1, T2 comprising, from 5′ to 3′:
(i) a sequence of the sense strand of a third RERS, and
(ii) a sequence at least substantially complementary to A1;
(e) amplifying a third single-stranded nucleic acid fragment (A2) using A1 as a template; and
(f) characterizing at least one of the single-stranded fragments of step (c) to identify the genetic variation.

106. The method of claim 105 wherein the first, second and third RERS are identical to each other.

107. A method for identifying a genetic variation at a defined location in a target nucleic acid, comprising
(a) forming a mixture of a first oligonucleotide primer (ODNP), a second ODNP and the target nucleic acid, wherein
(i) if the target nucleic acid is a double-stranded nucleic acid having a first strand and a second strand, then
the first ODNP comprises a nucleotide sequence at least substantially complementary to a nucleotide sequence of the first strand of the target nucleic acid located 3′ to the complement of the genetic variation, and
the second ODNP comprises a nucleotide sequence at least substantially complementary to a nucleotide sequence of the second strand of the target nucleic acid located 3′ to the genetic variation,
or
(ii) if the target nucleic acid is a single-stranded nucleic acid, then
the first ODNP comprises a nucleotide sequence at least substantially identical to a nucleotide sequence of the target nucleic acid located 5′ to the genetic variation, and
the second ODNP comprises a nucleotide sequence at least substantially complementary to a nucleotide sequence of the target nucleic acid located 3′ to the genetic variation,
the first and the second ODNPs each further comprising a nucleotide sequence of the sense strand of a nicking endonuclease recognition sequence (NERS);
(b) extending the first and the second ODNPs to produce an extension product comprising two NERSs;
(c) exponentially amplifying single-stranded nucleic acid fragments using the extension product of step (b) as a template in the presence of one or more nicking endonucleases (NEs) that recognizes the NERS(s), wherein the single-stranded nucleic acid fragments have no more than 25 nucleotides; and
(d) characterizing at least one of the single-stranded fragments of step (c) to thereby identify the genetic variation.

108. The method of claim 107 wherein the NERS in the first ODNP is identical to the NERS in the second ODNP.

109. The method of claim 107 wherein the genetic variation is a single nucleotide polymorphism.

110. The method of claim 107 wherein the genetic variation is associated with a disease.

111. The method of claim 107 wherein the disease is a human genetic disease.

112. The method of claim 107 wherein the genetic variation is associated with drug resistance of a pathogenic microorganism.

113. The method of claim 108 wherein the nicking agent is N.BstNB I.

114. The method of claim 107 wherein step (c) is performed under an isothermal condition.

115. The method of claim 114 wherein step (c) is performed at 50° C.-70° C.

116. The method of claim 107 wherein step (c) is performed in the presence of a DNA polymerase selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, and T4 DNA polymerase homoenzyme.

117. The method of claim 116 wherein the DNA polymerase is exo⁻ Vent, exo⁻ Deep Vent, exo⁻ Bst, exo⁻ Bca, or 90° Nm™ DNA polymerase.

118. The method of claim 107 wherein the amplified single-stranded fragments of step (c) contains no more than 17 nucleotides.

119. The method of claim 118 wherein the amplified single-stranded fragment of step (c) contains no more than 12 nucleotides.

120. The method of claim 119 wherein the amplified single-stranded fragment of step (c) contains no more than 8 nucleotides.

121. The method of claim 107 wherein the characterizing of step (d) is performed at least partially by the use of a technique selected from the group consisting of mass spectrometry, liquid chromatography, fluorescence polarization, and electrophoresis.

122. The method of claim 121 wherein the characterizing of step (d) is performed at least partially by the use of liquid chromatography.

123. The method of claim 121 wherein the characterizing of step (d) is performed at least partially by the use of mass spectrometry.

124. The method of claim 121 wherein the characterizing of step (d) is performed at least partially by both liquid chromatography and mass spectrometry.

125. The method of claim 107 wherein the first ODNP, the second ODNP or both are immobilized.

126. The method of claim 107 wherein the target nucleic acid is immobilized.

127. A method for identifying a genetic variation at a defined location in a target nucleic acid, comprising
(a) forming a mixture of a first oligonucleotide primer (ODNP), a second ODNP and the target nucleic acid, wherein
(i) if the target nucleic acid is a double-stranded nucleic acid having a first strand and a second strand, then
the first ODNP comprises a nucleotide sequence at least substantially complementary to a nucleotide sequence of the first strand of the target nucleic acid located 3′ to the complement of the genetic variation, and
the second ODNP comprises a nucleotide sequence at least substantially complementary to a nucleotide sequence of the second strand of the target nucleic acid located 3′ to the genetic variation,
or
(ii) if the target nucleic acid is a single-stranded nucleic acid, then
the first ODNP comprises a nucleotide sequence at least substantially identical to a nucleotide sequence of the target nucleic acid located 5′ to the genetic variation, and
the second ODNP comprises a nucleotide sequence at least substantially complementary to a nucleotide sequence of the target nucleic acid located 3′ to the genetic variation,
the first and the second ODNPs each further comprising a nucleotide sequence of a sense strand of a nicking endonuclease recognition sequence (NERS);
(b) extending the first and the second ODNPs to produce an extension product comprising two NERSs;
(c) amplifying a first single-stranded nucleic acid fragment using one strand of the extension product of step (b) as a template in the presence of one or more nicking endonucleases (NEs) that recognizes the NERS(s);
(d) providing a second single-stranded nucleic acid molecule (T2) to anneal to A1, T2 comprising, from 5′ to 3′:
(i) a sequence of the sense strand of a NERS, and
(ii) a sequence at least substantially complementary to A1;
(e) amplifying a third single-stranded nucleic acid fragment (A2) using A1 as a template; and
(f) characterizing the single-stranded fragment of step (c) to thereby identify the genetic variation,
wherein A1, A2 or both have at most 25 nucleotides.

128. The method of claim 127 wherein the NERSs in the first ODNP, the second ODNP and T2 are identical to each other.

129. The method of claim 127 wherein the genetic variation is a single nucleotide polymorphism.

130. The method of claim 127 wherein the genetic variation is associated with a disease.

131. The method of claim 127 wherein the disease is a human genetic disease.

132. The method of claim 127 wherein the genetic variation is associated with drug resistance of a pathogenic microorganism.

133. The method of claim 128 wherein the nicking agent is N.BstNB I.

134. The method of claim 127 wherein step (c) is performed under an isothermal condition.

135. The method of claim 134 wherein step (c) is performed at 50° C.-70° C.

136. The method of claim 127 wherein step (c) is performed in the presence of a DNA polymerase selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, and T4 DNA polymerase homoenzyme.

137. The method of claim 136 wherein the DNA polymerase is exo⁻ Vent, exo⁻ Deep Vent, exo⁻ Bst, exo⁻ Bca, or 9° Nm™ DNA polymerase.

138. The method of claim 127 wherein the amplified single-stranded fragments of step (c) contains no more than 17 nucleotides.

139. The method of claim 138 wherein the amplified single-stranded fragment of step (c) contains no more than 12 nucleotides.

140. The method of claim 139 wherein the amplified single-stranded fragment of step (c) contains no more than 8 nucleotides.

141. The method of claim 127 wherein the characterizing of step (d) is performed at least partially by the use of a technique selected from the group consisting of mass spectrometry, liquid chromatography, fluorescence polarization, and electrophoresis.

142. The method of claim 141 wherein the characterizing of step (d) is performed at least partially by the use of liquid chromatography.

143. The method of claim 141 wherein the characterizing of step (d) is performed at least partially by the use of mass spectrometry.

144. The method of claim 141 wherein the characterizing of step (d) is performed at least partially by both liquid chromatography and mass spectrometry.

145. The method of claim 127 wherein the first ODNP, the second ODNP or both are immobilized.

146. The method of claim 127 wherein the target nucleic acid is immobilized.

147. A method for determining the presence or the absence of a target nucleic acid in a sample, comprising
(A) forming a mixture comprising:
(i) the nucleic acid molecules of the sample;
(ii) a first single-stranded nucleic acid molecule (T1) comprising from 3′ to 5′:
(a) a first sequence that is at least substantially complementary to the target nucleic acid,
(b) a sequence of the antisense strand of a first nicking agent recognition sequence (NARS), and
(c) a second sequence having at most 25 nucleotides;
(iii) a second single-stranded nucleic acid molecule (T2) comprising from 3′ to 5′:
(a) a sequence that is at least substantially identical to the second sequence of T1, and
(b) a sequence of the sense strand of a second NARS; and
(iv) a first nicking endonuclease (NA) that recognizes the first NARS, a second NA that recognizes the second NARS, a DNA polymerase, and one or more deoxynucleoside triphosphate(s);
(B) maintaining the mixture at conditions that exponentially amplify a single-stranded nucleic acid molecule (A2) if the target nucleic acid is present in the sample; and
(C) detecting the presence or the absence of A2 to determine the presence, or the absence, of the target nucleic acid in the sample.

148. The method of claim 147 wherein the first NARS and the second NARS are identical and recognizable by a nicking endonuclease.

149. The method of claim 147 wherein the T1 is immobilized.

150. The method of claim 147 wherein the T2 is immobilized.

151. The method of claim 147 wherein the target nucleic acid is immobilized.

152. A method for determining the presence or the absence of a target nucleic acid in a sample, comprising
(A) form a mixture comprising:
(i) the nucleic acid molecules of the sample;
(ii) a first single-stranded nucleic acid molecule (T1) comprising from 3′ to 5′:
(a) a sequence that is at least substantially complementary to the target nucleic acid, and
(b) a sequence of the sense strand of a first nicking agent recognition sequence (NARS),
(iii) a second single-stranded nucleic acid molecule (T2) comprising from 3′ to 5′:
(a) a sequence that is at least substantially complementary to the sequence of T1 that is located 3′ to the sequence of the sense strand of the first NARS, and
(b) a sequence of the sense strand of a second NARS; and
(iv) a first nicking endonuclease (NA) that recognizes the first NARS, a second NA that recognizes the second NARS, a DNA polymerase, and one or more deoxynucleoside triphosphate(s);
(B) maintaining the mixture at conditions that amplify a single-stranded nucleic acid molecule (A2) that
(i) is at least substantially identical to the target nucleic acid, and
(ii) has at most 25 nucleotides if the target nucleic acid is present in the sample; and
(C) detecting the presence or the absence of A2 to determine the presence, or the absence, of the target nucleic acid in the sample.

153. The method of claim 152 wherein the first and second NARS are identical.

154. The method of claim 152 wherein the T1 is immobilized.

155. The method of claim 152 wherein the T2 is immobilized.

156. The method of claim 152 wherein the target nucleic acid is immobilized.

157. A method for determining the presence or absence of a target nucleic acid that comprises a first nicking endonuclease recognition sequence (NERS) in a sample, the method comprising:
(A) forming a mixture comprising:
(i) the nucleic acid molecules of the sample,
(ii) a single-stranded nucleic acid molecule (T2) comprising from 3′ to 5′:
(a) a sequence that is at least substantially identical to a portion of the target nucleic acid molecule located 5′ to the sequence of the antisense strand of the first NERS, and
(b) a sequence of the sense strand of a second NERS, and
(iii) a first nicking endonuclease (NE) that recognizes the first NERS; a second NE that recognizes the second NERS, a DNA polymerase, and one or more deoxynucleoside triphosphate(s);
(B) maintaining the mixture at conditions that exponentially amplify a single-stranded nucleic acid molecule (A2) if the target nucleic acid is present in the sample; and
(C) detecting the presence or absence of A2 to determine the presence or absence of the target nucleic acid in the sample.

158. The method of claim 157 wherein the first NERS is identical to the second NERS.

159. A method for determining the presence or absence of a target nucleic acid that comprises a first nicking endonuclease recognition sequence (NERS) in a sample, the method comprising:
(A) forming a mixture comprising:
(i) the target nucleic acid molecule,
(ii) a first single-stranded nucleic acid molecule (T1) that is substantially identical to one strand of the target nucleic acid and comprise a sequence of the antisense strand of the first NERS,
(iii) a second single-stranded nucleic acid molecule (T2) comprising from 3′ to 5′:
(a) a sequence that is at least substantially identical to a portion of T1 located 5′ to the sequence of the antisense strand of the first NERS, and
(b) a sequence of the sense strand of a second NERS, and
(iv) a first nicking endonuclease (NE) that recognizes the first NERS; a second NE that recognizes the second NERS, a DNA polymerase, and one or more deoxynucleoside triphosphate(s);
(B) maintaining the mixture at conditions that exponentially amplify a single-stranded nucleic acid molecule (A2) if the target nucleic acid is present in the sample; and
(C) detecting the presence or absence of A2 to determine the presence or absence of the target nucleic acid in the sample.

160. The method of claim 159 wherein the first NERS is identical to the second NERS.

161. The method of claim 159 wherein A2 has at most 25 nucleotides.

162. The method of claim 159 wherein the T1 is immobilized.

163. The method of claim 159 wherein the target nucleic acid is immobilized.

164. A method for determining the presence or absence of a target nucleic acid in a sample, comprising
(A) forming a mixture of a first oligonucleotide primer (ODNP), a second ODNP, and the nucleic acid molecules of the sample, wherein
(i) if the target nucleic acid is a double-stranded nucleic acid having a first strand and a second strand,
the first ODNP comprises a nucleotide sequence of a sense strand of a first restriction endonuclease recognition sequence (RERS) and a nucleotide sequence at least substantially complementary to a first portion of the first strand of the target nucleic acid, and
the second ODNP comprises a nucleotide sequence at least substantially complementary to a second portion of the second strand of the target nucleic acid and comprises a sequence of the sense strand of a second RERS, the second portion being located 3′ to the complement of the first portion in the second strand of the target nucleic acid,
or
(ii) if the target nucleic acid is a single-stranded nucleic acid, the first ODNP comprises a nucleotide sequence of a sense strand of a first RERS and a nucleotide sequence at least substantially identical to a first portion of the target nucleic acid, and
the second ODNP comprises a nucleotide sequence at least substantially complementary to a second portion of the target nucleic acid and comprises a sequence of the sense strand of a second RERS, the second portion being located 5′ to the first portion in the target nucleic acid;
(B) maintaining the mixture at conditions that, if the target nucleic acid is present in the sample, exponentially amplify a single-stranded nucleic acid fragment (A2) in the presence of restriction endonucleases (REs) that recognize the first RERS and the second RERS, deoxyribonucleoside triphosphates and at least one modified deoxyribonucleoside triphosphate, and a DNA polymerase, wherein A2 is no more than 25 nucleotides in length; and
(C) detecting the presence or absence of A2 to determine the presence or absence of the target nucleic acid in the sample.

165. The method of claim 164 wherein the first RERS is identical to the second RERS.

166. A method for determining the presence or absence of a target nucleic acid in a sample, comprising
(A) forming a mixture of a first oligonucleotide primer (ODNP), a second ODNP, and the nucleic acid molecules of the sample, wherein
(i) if the target nucleic acid is a double-stranded nucleic acid having a first strand and a second strand,
the first ODNP comprises a nucleotide sequence of a sense strand of a first nicking endonuclease recognition sequence (NERS) and a nucleotide sequence at least substantially complementary to a first portion of the first strand of the target nucleic acid, and
the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target nucleic acid,
or
(ii) if the target nucleic acid is a single-stranded nucleic acid, the first ODNP comprises a sequence of a sense strand of a first NERS and a nucleotide sequence at least substantially identical to a first portion of the target nucleic acid, and
the second ODNP comprises a nucleotide sequence at least substantially complementary to a second portion of the target nucleic acid and comprises a sequence of the sense strand of a second NERS, the second portion being located 5′ to the first portion in the target nucleic acid;
(B) maintaining the mixture at conditions that, if the target nucleic acid is present in the sample, exponentially amplify a single-stranded nucleic acid fragment (A2) in the presence of nicking endonucleases (NEs) that recognize the first NERS and the second NERS, deoxyribonucleoside triphosphates, and a DNA polymerase, wherein A2 is no more than 25 nucleotides in length; and
(C) detecting the presence or absence of A2 to determine the presence or absence of the target nucleic acid in the sample.

167. The method of claim 166 wherein the first RERS is identical to the second RERS.

168. A method for determining the presence or absence of a target nucleic acid in a sample, comprising
(A) forming a mixture of a first oligonucleotide primer (ODNP), a second ODNP, and the nucleic acid molecule of the sample, wherein
(i) if the target nucleic acid is a double-stranded nucleic acid having a first strand and a second strand,
the first ODNP comprises a nucleotide sequence of a sense strand of a first nicking endonuclease recognition sequence (NERS) and a nucleotide sequence at least substantially complementary to a first portion of the first strand of the target nucleic acid, and
the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target nucleic acid,
or
(ii) if the target nucleic acid is a single-stranded nucleic acid,
the first ODNP comprises a nucleotide sequence of a sense strand of a first NERS and a nucleotide sequence at least substantially identical to a first portion of the target nucleic acid, and
the second ODNP comprises a nucleotide sequence at least substantially complementary to a second portion of the target nucleic acid and comprises a sequence of the sense strand of a second NERS, the second portion being located 5′ to the first portion in the target nucleic acid;
(B) subjecting the mixture to conditions that, if the target nucleic acid is present in the sample,
(i) extend the first and the second ODNPs to produce an extension product comprising both the first and the second NERSs;
(ii) amplify a first single-stranded nucleic acid fragment (A1) using one strand of the extension product of step (b) as a template in the presence of one more nicking endonucleases (NEs) that recognizes the first and the second NERSs;
(iii) in the presence of a second single-stranded nucleic acid molecule (T2) capable of annealing to A1, amplify a third single-stranded nucleic acid fragment (A2) using A1 as a template, wherein A1, A2 or both have at most 25 nucleotides, and wherein T2 comprising, from 5′ to 3′:
(a) a sequence of the sense strand of a third NERS, and
(b) a sequence at least substantially complementary to A1; and
(C) detecting the presence or absence of A2 to determine the presence or absence of the target nucleic acid in the sample.

169. The method of claim 168 wherein the first, second and third NERSs are identical.

170. A method for determining the presence or absence of a target nucleic acid in a sample, comprising
(A) forming a mixture comprising:
(i) the nucleic acid molecules of the sample,
(ii) a single-stranded nucleic acid probe (T1) that comprises, from 3′ to 5′, a sequence that is at least substantially complementary to the 5′ portion of the target nucleic acid, and a sequence of the antisense strand of a first nicking agent recognition sequence (NARS),
(B) separating the probe molecules that have hybridized to the target nucleic acid, if any, from those that have not hybridized to the target nucleic acid;
(C) performing an amplification reaction in the presence of the probe molecules that have hybridized to the target nucleic acid, if any, and a first nicking agent (NA) that recognizes the first NARS;
(D) providing a single-stranded nucleic acid molecule (T2) comprising, from 5′ to 3′:
(i) a sequence of the sense strand of a second NARS, and
(ii) a sequence that is at least substantially identical to the portion of the first single-stranded nucleic acid probe located 5′ to the sequence of the antisense strand of the first NARS,
(E) performing an amplification reaction in the presence of a second NA that recognizes the second NARS;
(F) detecting the presence or absence of the amplification product of step (E) to determine the presence or absence of the target nucleic acid in the sample.

171. The method of claim 170 wherein the first and second NARSs are identical.

172. The method of claim 170 wherein the nucleic acid molecules of the sample are single-stranded or denatured to be single-stranded and immobilized via their 5′ termini.

173. The method of claim 170 wherein the nucleic acid molecules of the sample are immobilized.

174. The method of claim 170 wherein the single-stranded nucleic acid probe T1 is immobilized.

175. A method for determining the presence or absence of a target nucleic acid in a sample, comprising
(A) forming a mixture comprising:
(i) the nucleic acid molecules of the sample,
(ii) a partially double-stranded nucleic acid probe that comprises:
(a) a sequence of a sense strand of a first NARS, a sequence of an antisense of the first NARS, or both; and
(b) a 5′ overhang in the strand that the strand itself or an extension product thereof contains a nicking site (NS) nickable by a first nicking agent (NA) that recognizes the first NARS, or
a 3′ overhang in the strand that neither the strand nor an extension product thereof contains the NS,
wherein each overhang comprises a nucleic acid sequence at least substantially complementary to the target nucleic acid;
(B) separating the probe molecules that have hybridized to the target nucleic acid, if any, from those that have not hybridized to the target nucleic acid;
(C) performing an amplification reaction in the presence of the probe molecules that have hybridized to the target nucleic acid, if any, and a first nicking agent (NA) that recognizes the first NARS;
(D) providing a single-stranded nucleic acid molecule (T2) comprising, from 5′ to 3′:
(i) a sequence of the sense strand of a second NARS, and
(ii) a sequence that is at least substantially identical to the portion of the nucleic acid probe located 5′ to the sequence of the antisense strand of the first NARS,
(E) performing an amplification reaction in the presence of a second NA that recognizes the second NARS;
(F) detecting the presence or absence of the amplification product of step (E) to determine the presence or absence of the target nucleic acid in the sample.

176. The method of claim 175 wherein the first and second NARSs are identical.

177. The method of claim 175 wherein the nucleic acid molecules of the sample are immobilized.

178. A method for determining the presence or absence of a junction between two specific exons in a cDNA molecule, comprising:
(A) providing an at least partially double-stranded nucleic acid molecule (N1) comprising
(i) at least one of a sequence of the sense strand of a first nicking agent recognition sequence (NARS) and a sequence of the antisense strand of the first NARS, and
(ii) at least one strand of a portion of the cDNA molecule, the portion being suspected to contain the junction between the two exons;
(B) amplifying a first single-stranded nucleic acid molecule (A1) in the presence of a nicking agent (NA) that recognizes the first NARS, a DNA polymerase, and one or more deoxynucleoside triphosphate(s), wherein the amplifying uses the portion of the cDNA as a template for the polymerase;
(C) providing a second single-stranded nucleic acid molecule (T2) comprising, from 5′ to 3′:
(i) a sequence of the sense strand of a second NARS, and
(ii) a sequence that is at least substantially complementary to A1;
(D) amplifying a third single-stranded nucleic acid molecule (A2) in the presence of T2, A1, the first NA, a second NA that recognizes the second NARS, the DNA polymerase, and the dexoynucleoside triphosphate(s), wherein A2 is at least substantially complementary to A1; and
(E) detecting and/or characterizing A2 to determine the presence or absence of the junction in the cDNA molecule.

179. The method of claim 178 wherein the first NARS is identical to the second NARS.

180. The method of claim 178 wherein both the first and the second NAs are nicking endonucleases (NEs).

181. The method of claim 180 wherein both the first and the second NAs are N.BstNB I.

182. The method of claim 179 wherein both the first and second NAS are a nicking endonuclease (NE).

183. The method of claim 178 wherein steps (A)-(D) are performed in a single vessel.

184. The method of claim 178 wherein N1 comprises the sequence of the antisense strand of the first NARS.

185. The method of claim 178 wherein N1 comprises the sequence of the sense strand of the first NARS.

186. The method of claim 185 wherein both the first and the second NAs are restriction endonucleases (REs), and at least one of the nucleoside triphosphate(s) is modified.

187. The method of claim 178 wherein A1 is from 8 to 24 nucleotides in length.

188. The method of claim 187 wherein A1 is from 12 to 17 nucleotides in length.

189. The method of claim 178 wherein A2 is from 8 to 24 nucleotides in length.

190. The method of claim 189 wherein A2 is from 12 to 17 nucleotides in length.

191. The method of claim 178 wherein each of steps (B) and (D) is performed under isothermal conditions.

192. The method of claim 191 wherein each of steps (B) and (D) is performed at 50° C.-70° C.

193. The method of claim 178 wherein the DNA polymerase is 5′→3′ exonuclease deficient.

194. The method of claim 193 wherein the 5′→3′ exonuclease deficient DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ polymerase and T4 DNA polymerase homoenzyme.

195. The method of claim 194 wherein the 5′→3′ exonuclease deficient DNA polymerase is exo⁻ Bst polymerase, exo⁻ Bca polymerase, exo⁻ Vent polymerase, exo⁻ Deep Vent polymerase, or 90° Nm™ polymerase.

196. The method of claim 178 wherein the DNA polymerase has a strand displacement activity.

197. The method of claim 178 wherein each of steps (B) and (D) is performed in the presence of a strand displacement facilitator.

198. The method of claim 197 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

199. The method of claim 198 wherein the strand displacement facilitator is trehalose.

200. The method of claim 178 wherein step (E) is performed at least partially by the use of a technique selected from the group consisting of mass spectrometry, liquid chromatography, fluorescence polarization, and electrophoresis.

201. The method of claim 200 wherein step (E) is performed at least partially by the use of liquid chromatography.

202. The method of claim 200 wherein step (E) is performed at least partially by the use of mass spectrometry.

203. The method of claim 178 wherein the N1 is immobilized.

204. The method of claim 203 wherein the T2 is immobilized.

205. A method for determining the presence or absence of a junction between two exons in a cDNA molecule, wherein the junction, if present, is located 5′ to a sequence of the antisense strand of a first nicking endonuclease recognition sequence (NERS) in the cDNA molecule, the method comprising:
(A) forming a mixture comprising:
(i) the cDNA molecule,
(ii) a single-stranded nucleic acid molecule (T2) comprising from 3′ to 5′:
(a) a sequence that is at least substantially identical to a portion of the cDNA molecule located 5′ to the sequence of the antisense strand of the first NERS, and
(b) a sequence of the sense strand of a second NERS, and
(iii) a first nicking endonuclease (NE) that recognizes the first NERS; a second NE that recognizes the second NERS, a DNA polymerase, and one or more deoxynucleoside triphosphate(s);
(B) maintaining the mixture at conditions that exponentially amplify a single-stranded nucleic acid molecule (A2); and
(C) characterizing A2 to determine the presence or absence of the junction in the cDNA molecule.

206. The method of claim 205 wherein the first NERS is identical to the second NERS.

207. A method for determining the presence or absence of a junction between an upstream exon (Exon A) and a downstream exon (Exon B) of a gene in a cDNA molecule, comprising
(A) forming a mixture of a first oligonucleotide primer (ODNP), a second ODNP, and the cDNA molecule, wherein
(i) the first ODNP comprises a sequence at least substantially complementary to a portion of the antisense strand of Exon A near the 5′ terminus of Exon A in the antisense strand,
(ii) the second ODNP comprises a sequence at least substantially complementary to a portion of the sense strand of Exon B near the 5′ terminus of Exon B in the sense strand, and
(iii) at least one of the first ODNP and the second ODNP further comprises a sequence of a sense strand of a first nicking agent recognition sequence (NARS); and
(B) performing a first amplification reaction in the presence of a nicking agent (NA) that recognizes the first NARS under the conditions that amplify a first single-stranded nucleic acid (A1) if both Exon A and Exon B are present in the cDNA;
(C) providing a second single-stranded nucleic acid molecule (T2) comprising, from 5′ to 3′:
(i) a sequence of the sense strand of a second NARS, and
(ii) a sequence at least substantially complementary to A1;
(D) performing a second amplification reaction in the presence of a second NA that recognizes the second NARS under the conditions that amplify a third single-stranded nucleic acid fragment (A2) using A1 as a template if both Exon A and Exon B are present in the cDNA molecule; and
(G) detecting and/or characterizing A2 to determine the presence or absence of the junction between Exon A and Exon B in the cDNA molecule.

208. The method of claim 207 wherein the first NARS is identical to the second NARS.

209. The method of claim 207 wherein the cDNA molecule is immobilized.

210. The method of claim 207 wherein the first ODNP, the second ODNP or both are immobilized.

211. A method for determining the presence or absence of a junction between an upstream exon (Exon A) and a downstream exon (Exon B) of a gene in a cDNA molecule, comprising
(A) forming a mixture of a first oligonucleotide primer (ODNP), a second ODNP, and the cDNA molecule, wherein
(i) the first ODNP comprises
(a) a sequence at least substantially complementary to a portion of the antisense strand of Exon A near the 5′ terminus of Exon A in the antisense strand, and
(b) a sequence of the sense strand of a first nicking agent recognition sequence (NARS); and
(ii) the second ODNP comprises
(a) a sequence at least substantially complementary to a portion of the sense strand of Exon B near the 5′ terminus of Exon B in the sense strand, and
(b) a sequence of the sense strand of a second NARS;
(B) performing a first amplification reaction in the presence of a first nicking agent (NA) that recognizes the first NARS and a second NA that recognizes the second NARS under the conditions that amplify a first single-stranded nucleic acid (A1) if both Exon A and Exon B are present in the cDNA;
(C) providing a second single-stranded nucleic acid molecule (T2) comprising, from 5′ to 3′:
(i) a sequence of the sense strand of a third NARS, and
(ii) a sequence at least substantially complementary to A1;
(D) performing a second amplification reaction in the presence of a third NA that recognizes the second NARS under the conditions that amplify a third single-stranded nucleic acid fragment (A2) using A1 as a template if both Exon A and Exon B are present in the cDNA molecule; and
(E) detecting and/or characterizing A2 to determine the presence or absence of the junction between Exon A and Exon B in the cDNA molecule.

212. The method of 211 wherein the first, second and third NARS are identical.

213. A method for determining the presence or absence of a junction between an upstream exon (Exon A) and a downstream exon (Exon B) of a gene in a cDNA molecule, comprising
(A) forming a mixture of a first oligonucleotide primer (ODNP), a second ODNP, and the cDNA molecule, wherein
(i) the first ODNP comprises
(a) a sequence at least substantially complementary to a portion of the antisense strand of Exon A near the 5′ terminus of Exon A in the antisense strand, and
(b) a sequence of the sense strand of a first nicking agent recognition sequence (NARS); and
(ii) the second ODNP comprises
(a) a sequence at least substantially complementary to a portion of the sense strand of Exon B near the 5′ terminus of Exon B in the sense strand, and
(b) a sequence of the sense strand of a second NARS;
(B) maintaining the mixture at conditions that, if both Exon A and Exon B are present in the cDNA molecule, exponentially amplify a single-stranded nucleic acid fragment (A2); and
(C) detecting and/or characterizing A2 to determine the presence or absence of the junction between Exon A and Exon B in the cDNA molecule.

214. The method of claim 213 wherein the first and second NERSs are identical.

215. The method of claim 213 wherein the cDNA molecule is immobilized.

216. The method of claim 213 wherein the first ODNP, the second ODNP or both are immobilized.
